(19) United States
(12) Patent Application Publication
(10) Pub. No.: US 2008/0293758 A1
(43) Pub. Date: Nov. 27, 2008

(54) Combination of a Steroid Sulfatase Inhibitor and an Ascomycin

(75) Inventor: Josef Gottfried Meingassner, Perchtoldsdorf (AT)

Correspondence Address: MONTGOMERY, MCCRACKEN, WALKER & RHOADS, LLP, 123 SOUTH BROAD STREET, AVENUE OF THE ARTS, PHILADELPHIA, PA, 19109, US

(73) Assignee: Novartis AG, Basel (CH)

(21) Appl. No.: 11/817,799

(22) PCT Filed: Mar. 15, 2006
(86) PCT No.: PCT/EP2006/002383

§ 371 Date: Sep. 5, 2007

(30) Foreign Application Priority Data
Mar. 17, 2005 (GB) 0505539.7

(51) Int. Cl. A61K 31/436; A61P 29/00

(52) U.S. Cl. 514/291

(57) ABSTRACT

A combination of a steroid sulfatase inhibitor and an ascomycin, which combination is useful as a pharmaceutical.

The present invention relates to a combination of a steroid sulfatase inhibitor and an ascomycin.

In one aspect the present invention provides a combination of a steroid sulfatase inhibitor and an ascomycin.

An ascomycin (ascomycins) includes ascomycin as such, and derivatives thereof. A derivative is to be understood as being an antagonist, agonist or analogue of the parent compound which retains the basic structure and modulates at least one of the biological, for example immunological, properties of the parent compound. e.g. obtainable by fermentation techniques. Such derivatives are e.g. obtainable by chemical derivatisation or fermentation manipulation procedures of naturally occurring ascomycins.

Appropriate ascomycins are hereinafter designated as “ascomycin(s) of (according to) the present invention” and e.g. include compounds as disclosed in U.S. Pat. No. 3,244,592, EP349061, EP184162, EP315978, EP323042, EP423714, EP427680, EP465426, EP474126, WO9113889, WO9119495, EP484936, EP523088, EP532089, EP569337, EP626385, WO935059, WO978182;

such as
ascomycin;
tacrolimus (FK506; Prograf^{R});
imidazolylmethoxyascomycin (WO978182, compound of formula I, e.g. of Example 1);
32-O-(1-hydroxyethylindol-5-yl)ascomycin (L-732531) (Transplantation 65 [1998] 10-18, 18-26, on page 11, FIG. 1;
(32-desoxy,32-epi-N1-tetrazolyl)ascomycin (ABT-281) (J. Invest. Dermatol. 12 [1999] 729-738, on page 730, FIG. 1);
{1R,5Z,9S,12S-[1E-(1R,3R,4R)],13R,14S,17R,18E,21S,23S,24R,25S,27R}-17-ethyl-1,14-dihydroxy-12-[2-(4-hydroxy-3-methoxycyclohexyl)-1-methylvinyl]-23,25-dimethoxy-13,19,21,27-tetramethyl-11,28-dioxa-4-azatricyclo[22.3.1.0(4,9)]octacos-5,18-diene-2,3,10,16-tetraone (Example 8 in EP626385), hereinafter referred to as “5,6-dehydroascomycin”;
{1E-(1R,3R,4R)]1R,4S,5R,6S,9R,10E,13S,15S,16R,17S,19S,20S}-9-ethyl-6,16,20-trihydroxy-4-[2-(4-hydroxy-3-methoxycyclohexyl)-1-methylvinyl]-15,17-dimethoxy-5,11,13,19-tetramethyl-3-oxa-22-azatricyclo[18.6.1.0(1,22)]heptacos-10-ene-2,8,21,27-tetraone (Examples 6d and 71 in EP569337), hereinafter referred to as “ASD 732”; and
pimecrolimus (INN recommended) (ASM981; Elidel™), i.e {[1E-(1R,3R,4S)]1R,9S,12S,13R,14S,17R,18E,21S,23S,24R,25S,27R}-12-[2-(4-chloro-3-methoxycyclohexyl)-1-methylvinyl]-17-ethyl-1,14-dihydroxy-23,25-dimethoxy-13,19,21,27-tetramethyl-11,28,dioxa-4-azatricyclo[22.3.1.0(4,9)]octacos-18-ene-2,3,10,16-tetraone of formula

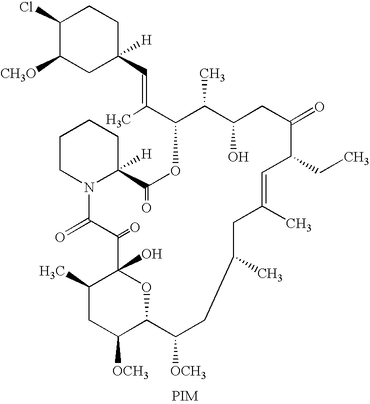

such as disclosed in EP 427 680 (33-epi-33-chloro-FR 520 of example 66a.

Appropriate steroid sulfatase inhibitors are hereinafter designated as “steroid sulfatase inhibitors of (according to) the present invention” and e.g. include compounds of formula

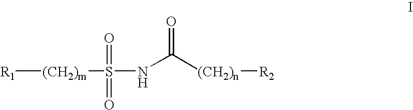

wherein

R₁ is (C₁₋₆)haloalkyl, unsubstituted (C₂₋₆)alkenyl, (C₂₋₆)alkenyl substituted by phenyl, unsubstituted or by 1 to 5 substitutents substituted
thienyl, pyridine, benzthiazolyl, chromanyl (i.e. 1,2-dihydrobenzopyranyl) or (C₆₋₁₈)aryl, wherein the substituents are selected from the group consisting of
halogen, nitro, di(C₁₋₄)alkylamino, cyano, (C₁₋₆)alkyl, (C₁₋₄)haloalkyl, unsubstituted phenylcarbonylamino(C₁₋₄)alkyl, (C₁₋₄)alkoxy, (C₁₋₄)haloalkoxy, aminocarbonyl, di(C₁₋₄)alkylaminocarbonyl, (C₁₋₄)alkylcarbonyl, (C₁₋₄)alkoxycarbonyl, unsubstituted phenyl, carboxyl, and phenyl-substituted phenylcarbonylamino(C₁₋₄)alkyl or substituted phenyl, wherein the phenyl-substitutents are selected from the group consisting of
   halogen, nitro, di(C₁₋₄)alkylamino, cyano, (C₁₋₆)alkyl, (C₁₋₄)haloalkyl, (C₁₋₄)alkoxy, (C₁₋₄)haloalkoxy, aminocarbonyl, di(C₁₋₄)alkylaminocarbonyl, (C₁₋₄)alkylcarbonyl, (C₁₋₄)alkoxycarbonyl and carboxyl, or

R₁ is a group of formula

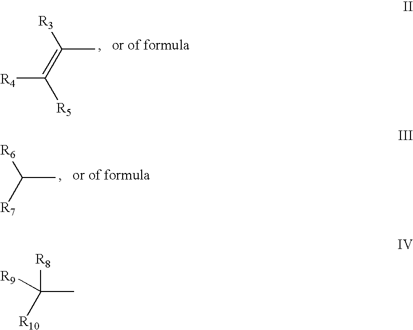

R₂ is a group of formula

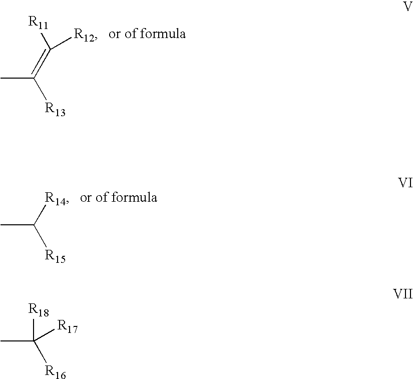

R₃ and R₁₃ independently of each other are hydrogen, hydroxy, halogen, cyano, (C₁₋₄)alkyl, (C₁₋₄)alkoxy, phenyl or phenoxy,

at least one of
R₄ and R₅ together with the carbon atom to which they are attached,
R₁₁ and R₁₂ together with the carbon atom to which they are attached,

independently of each other are a substituted
bridged cycloalkyl system,
(C₄₋₈)cycloalkyl,
piperidine, tetrahydropyridine, or
bridged heterocyclic system,

wherein the substitutents are selected from the group consisting of
(C₁₋₆)alkoxycarbonylamino,
(C₁₋₆)alkoxycarbonyl((C₁₋₄)alkyl)amino,
(C₁₋₆)alkoxycarbonyl((C₂₋₄)alkenyl)amino,
(C₃₋₈)cycloalkylcarbonylamino,
(C₃₋₈)cycloalkylcarbonyl((C₁₋₄)alkyl)amino,
(C₃₋₈)cycloalkylcarbonyl((C₂₋₄)alkenyl)amino,
(C₁₋₆)alkoxycarbonyloxy,
phenyl(C₁₋₄)alkylcarbonyloxy, wherein phenyl is unsubstituted or substituted and wherein the substituents are as defined above for substituted phenyl,
phenylsulphonyl, wherein phenyl is unsubstituted or substituted and wherein the substituents are defined as above for substituted phenyl,
(C₄₋₈)alkyl, e.g. (C₅₋₈)alkyl,
(C₁₋₄)hydroxyalkyl,
(C₁₋₄)hydroxyalkyl substituted by phenyl, wherein phenyl is unsubstituted or substituted and wherein the substituents are as defined above for substituted phenyl,
(C₁₋₆)alkoxycarbonyl(C₁₋₄)alkyl,
(C₃₋₈)cycloalkoxycarbonyl(C₁₋₄)alkyl,
(C₁₋₆)alkoxycarbonylamino(C₁₋₄)alkyl,
(C₃₋₈)cycloalkylcarbonylamino(C₁₋₄)alkyl,
phenyl or substituted phenyl, wherein the substituents are as defined above for substituted phenyl,
heterocyclyl having 5- or 6-ring members and 1 to 4 heteroatoms selected from N, O, S, e.g. oxadiazolyl,
(C₃₋₈)cycloalkoxycarbonyl,
(C₃₋₈)cycloalkyl(C₁₋₄)alkylcarbonyl, wherein cycloalkyl is unsubstituted or substituted by hydroxy,
phenylcarbonyl, wherein phenyl is unsubstituted or substituted and wherein the substituents are defined as above for substituted phenyl,
(C₃₋₈)cycloalkylaminocarbonyl,
(C₃₋₈)cycloalkyl((C₁₋₄)alkyl)aminocarbonyl,
(C₃₋₈)cycloalkyl((C₂₋₄)alkenyl)aminocarbonyl, and
(C₁₋₈)alkoxycarbonyl,

R₃, R₈, R₁₃ and R₁₈ independently of each other are hydrogen, hydroxy, halogen, cyano, (C₁₋₄)alkyl, (C₁₋₄)alkoxy, phenyl or phenoxy,

EITHER

R₈ or R₁₈, respectively, independently of each other are hydrogen, hydroxy, halogen, cyano, (C₁₋₄)alkyl, (C₁₋₄)alkoxy, phenyl or phenoxy, and at lest one of
R₉ and R₁₀ together with the carbon atom to which they are attached,
R₁₆ and R₁₇ together with the carbon atom to which they are attached,

independently of each other have the meaning of R₄ and R₅ together with the carbon atom to which they are attached, as defined above,

OR

at least one of
R₉ and R₁₀ together with the carbon atom to which they are attached,
R₁₆ and R₁₇ together with the carbon atom to which they are attached,

are (C₃₋₈)cycloalkyl, and

R₈ or R₁₈, respectively, independently of each other are a substituted
bridged cycloalkyl system, (C₄₋₈)cycloalkyl, substituted piperidine, tetrahydropyridine, or a bridged heterocyclic system,

wherein the substitutents are as defined above for the corresponding groups,

R₆ and R₁₅ independently of each other are (C₁₋₆)haloalkyl, unsubstituted or substituted (C₆₋₁₈)aryl, wherein the aryl-substitutents are as defined above, or a substituted
bridged cycloalkyl system, (C₄₋₈)cycloalkyl, piperidine, tetrahydropyridine, or bridged heterocyclic system,

wherein the substitutents are as defined above for the corresponding groups, or

R₆ and R₁₅ independently of each other are amino substituted by a substituted
bridged cycloalkyl system, (C₄₋₈)cycloalkyl, piperidine, tetrahydropyridine, or bridged heterocyclic system,

wherein the substitutents are as defined above for the corresponding group,

R₇ and R₁₄ independently of each other are a substituted
bridged cycloalkyl system, (C₄₋₈)cycloalkyl, piperidine, tetrahydropyridine, or bridged heterocyclic system, wherein the substitutents are as defined above for the corresponding groups,

or R₇ and R₁₄ independently of each other are amino substituted by a substituted
bridged cycloalkyl system, (C₄₋₈)cycloalkyl, piperidine, tetrahydropyridine, or bridged heterocyclic system,

wherein the substitutents are as defined above for the corresponding group,

m is 0, 1, 2, 3 or 4, such as 0 or 1,

n is 0, 1, 2, 3 or 4, such as 0 or 1, and

IF

m and/or n are other than 0,

THEN
R₁, if m is other than 0, and R₂, if n is other than 0, independently of each other have the meaning as defined above and additionally may be substituted piperazine, wherein the substitutents are as defined above for substituted piperidine above; and
a substituted bridged cycloalkyl system is substituted as defined above for a substituted bridged cycloalkyl system, and additionally may be substituted by oxo and/or (C₁₋₄)alkyl; and

IF

R₁ is a substituted
bridged cycloalkyl ring system, (C₄₋₈)cycloalkyl, piperidine, tetrahydropyridine, or a bridged heterocyclyl ring system, wherein the substituents are as defined above for the corresponding groups, or if R₁ is additionally piperazine, if m is other than 0,

THEN

R₂ has the meaning as defined above and additionally may be (C₁₋₆)haloalkyl, unsubstituted (C₂₋₆)alkenyl, (C₂₋₆)alkenyl substituted by phenyl, unsubstituted or by 1 to 5 substitutents substituted
thienyl, pyridine, benzthiazolyl, chromanyl (i.e. 1,2-dihydrobenzopyranyl) or (C₆₋₁₈)aryl,

wherein the substituents are as defined above for the corresponding groups, and

IF

m is 0, n is 0 and R₂ is substituted (C₄₋₈)cycloalkyl or a substituted bridged cycloalkyl ring system, wherein the substituents are as defined above,

THEN

R₁ is other than (C₁₋₆)haloalkyl; and

IF

m is 0, n is 0 and R₁ and/or R₂ are substituted (C₄₋₈)cycloalkyl,

THEN

(C₄₋₈)cycloalkyl is substituted as defined above with the exception of phenyl and substituted phenyl as a substituent,

with the proviso that

in a compound of formula I at least one substituent selected from the group consisting of a substituted bridged cycloalkyl ring system, substituted (C₄₋₈)cycloalkyl, substituted piperidine, substituted tetrahydropyridine, substituted piperazine, or a substituted bridged heterocyclyl ring system, wherein the substituents are as defined above for the corresponding groups, is present.

In a compound of formula I m is preferably 0 or 1, and n is preferably 0 or 1.

If not otherwise specified herein
cycloalkyl includes e.g. non-bridged (C₃₋₈)cycloalkyl, such as (C₄₋₈)cycloalkyl,
heterocyclyl includes heterocyclyl having 5 to 6 ring members and 1 to 4 heteroatoms selected from N, S or O, optionally anellated with another ring (system), such as piperidine, tetrahydropyridine, pyridine, piperazine, thienyl, pyridine, benzthiazolyl, chromanyl, oxadiazolyl,
aryl includes (C₆₋₁₈)aryl, e.g. (C₆₋₁₂)aryl, such as naphthyl, phenyl.

A substituent attached to cyclohexyl, a piperidine, tetrahydropyridine or piperazine ring in a compound of formula I may be in any position with respect to the sulfonamide group, or with respect to a group —(CH₂)ₘ— or —(CH₂)ₙ—, also attached to said ring, e.g. in 2, 3 or 4 position; and is preferably in 3 or in 4 position.

A bridged cycloalkyl system includes bridged (C₅₋₁₂)cycloalkyl, such as (C₆₋₈)cycloalkyl, wherein the bridge optionally comprises a heteroatom, such as N, e.g. including cycloalkyl anellated with another ring system, e.g. anellated with a (C₅₋₁₂)cycloalkyl, such as decalin and/or phenyl, e.g. including
decalin bridged by alkyl, e.g. methyl, such as adamantyl,
cyclohexyl or cycloheptyl, bridged by (C₁₋₄)alkyl, e.g. bridged by a —CH₂—CH₂— group,
cycloheptyl or cyclooctyl bridged by an amine group,
cyclohexyl or cycloheptyl bridged by an alkyl chain, e.g. (C₂₋₄)alkyl chain interrupted by a hetero atom, such as nitrogen, e.g. a —CH₂—NH—CH₂— group,
cycloheptyl bridged by an alkyl chain, e.g. (C₂₋₄)alkyl chain, which is interrupted by a hetero atom, such as nitrogen, e.g. a —CH₂—NH—CH₂— group and which bridged cycloheptyl is further anellated with phenyl.

A bridged substituted bridged heterocyclic system includes a bridged piperidine, e.g. bridged by (C₁₋₄)alkylene, such as ethylene.

Naphthyl includes e.g. naph-1-yl, naphth-2-yl, e.g. unsubstituted or substituted by di(C₁₋₄)alkylamino. Thiophenyl, includes e.g. thiophen-2-yl and thiophen-3-yl, e.g. substituted by 1 to 3 halogen. Benzthiazolyl, e.g. includes benzthiazol-2-yl, e.g. substituted by (C₁₋₄)alkoxy. Chromanyl, e.g. includes chroman-6-yl, e.g, substituted by (C₁₋₄)alkyl. Pyridine includes pyridine substituted by halogen and is bound to the (optionally (CH₂)_{m or n})carbonyl or (optionally (CH₂)_{m or n})sulfonyl group in a compound of formula I via a carbon atom.

A steroid sulfatase inhibitor of the present invention includes compound of formula I, wherein at least one of
R₄ and R₅ together with the carbon atom to which they are attached,
R₉ and R₁₀ together with the carbon atom to which they are attached,
R₁₁ and R₁₂ together with the carbon atom to which they are attached, or
R₁₆ and R₁₇ together with the carbon atom to which they are attached,
R₆,
R₇,
R₁₄, or
R₁₅

is substituted (C₄₋₈)cycloalkyl, wherein the substituents are as defined above for substituted cycloalkyl, with the exception of phenyl and substituted phenyl as a substituent, and the other substitutents are as defined above, such as a compound of formula I_{P2}, I_{P6}, I_{P7} or I_{P10} as defined below.

A steroid sulfatase inhibitor of the present invention also includes a compound of formula I, wherein at least one of
R₄ and R₅ together with the carbon atom to which they are attached,
R₉ and R₁₀ together with the carbon atom to which they are attached,
R₁₁ and R₁₂ together with the carbon atom to which they are attached, or
R₁₆ and R₁₇ together with the carbon atom to which they are attached,
R₆,
R₇,
R₁₄, or
R₁₅

is substituted piperidine, substituted tetrahydropyridine, or a substituted bridged heterocyclic system, and, if m is other than 0 and/or n is other than 0, additionally may be substituted piperazine, wherein the substituents are as defined above for substituted piperidine, substituted tetrahydropyridine, a substituted bridged heterocyclic system and wherein piperazine is substituted by groups as defined for substituted piperidine, and the other substitutents are as defined above, such as a compound of formula I_{P1}, I_{P4}, I_{P5}, I_{P8}, I_{P9}, I_{P12}, I_{P13} or I_{P14} as defined below.

A steroid sulfatase inhibitor of the present invention also includes a compound of formula I, which is a compound of formula

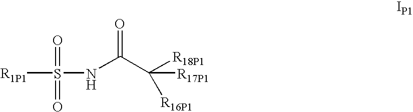

wherein R_{1P1} has the meaning as defined in R₁ above, and R_{16P1} and R_{17P1} together with the carbon atom to which they are attached are substituted piperidine or substituted tetrahydropyridine, wherein the substituents are as defined above for substituted piperidine.

In a compound of formula I_{P1} preferably

R_{1P1} is substituted or unsubstituted thienyl, benzthiazolyl, chromanyl, phenyl or naphthyl,

R_{16P1} and R_{17P1} together with the carbon atom to which they are attached are piperidine or tetrahydropyridine, preferably piperidine, substituted

a) at the nitrogen atom of the ring by substituents selected from the group consisting of
(C₁₋₆)alkoxycarbonyl, e.g. BOC (i.e. tert.butoxycarbonyl),
(C₁₋₆)alkoxycarbonyl(C₁₋₄)alkyl, e.g. tert.butoxycarbonylmethyl,
unsubstituted or substituted phenyl, wherein the substituents are as defined for phenyl above,
(C₁₋₆)alkylcarbonyl or phenylcarbonyl, (C₃₋₈)cycloalkyl(C₁₋₄)alkylcarbonyl,
heterocyclyl, e.g. pyridine, such as pyridin-2-yl, e.g. substituted by nitro,
more preferably piperidine substituted at the nitrogen atom by BOC, or unsubstituted or substituted phenyl,

and optionally

b) further substituted at a carbon atom of the ring by (C₁₋₄)alkyl,

and

R_{18P1} is hydrogen, phenyl or (C₁₋₄)alkyl, more preferably hydrogen or phenyl.

A steroid sulfatase inhibitor of the present invention also includes a compound of formula I, which is a compound of formula

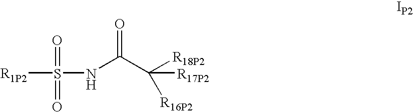

wherein R_{1P2} has the meaning of R₁ as defined above, R_{16P2} and R_{17P2} together with the carbon atom to which they are attached are substituted (C₄₋₇)cycloalkyl, wherein the substituents are as defined above for substituted cycloalkyl with the exception of phenyl or substituted phenyl as a substituent, and R_{18P2} has the meaning of R₁₈ as defined above.

In a compound of formula I_{P2} preferably
R_{1P2} is substituted or unsubstituted phenyl, naphthyl, alkenyl (e.g. substituted by phenyl), or thienyl.
R_{16P2} and R_{17P2} together with the carbon atom to which they are attached are cyclohexyl substituted by
(C₁₋₆)alkoxycarbonylamino(C₁₋₄)alkyl, (C₁₋₆)alkoxycarbonylamino, (C₁₋₆)alkoxycarbonyl-((C₁₋₄)alkyl)amino, (C₁₋₆)alkoxycarbonyl((C₂₋₄)alkenyl)amino, (C₃₋₈)cycloalkylcarbonyl-((C₁₋₄)alkyl)amino, (C₃₋₈)cycloalkylcarbonylamino(C₁₋₄)alkyl, (C₁₋₆)alkylcarbonylamino-(C₁₋₄)alkyl, (C₃₋₈)cycloalkyl(C₁₋₄)alkyl-carbonyloxy, (C₃₋₈)cycloalkyl(C₁₋₄)alkylcarbonyloxy, (C₃₋₈)cycloalkyl((C₁₋₄)alkyl)aminocarbonyl, phenylcarbonyl, or heterocyclyl having 5- or 6-ring members and 1 to 4 heteroatoms selected from N, O, S, e.g. oxadiazolyl, more preferably substituted by (C₁₋₆)alkoxycarbonylamino(C₁₋₄)alkyl or (C₁₋₆)alkoxycarbonylamino,

R_{18P2} is hydrogen

A steroid sulfatase inhibitor of the present invention also includes a compound of formula I, which is a compound of formula

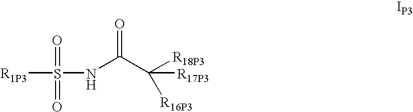

wherein R_{1P3} has the meaning of R₁ as defined above, R_{16P3} and R_{17P3} together with the carbon atom to which they are attached are a substituted bridged cycloalkyl ring system, wherein the substituents are as defined above for a bridged cycloalkyl ring system, and R_{18P3} has the meaning of R₁₈ as defined above.

In a compound of formula I_{P3} preferably
R_{1P3} is unsubstituted or substituted phenyl or thienyl.
R_{16P3} and R_{17P3} together with the carbon atom to which they are attached are a bridged cycloalkyl ring system which is substituted by
   (C₄₋₁₂)alkyl,
   (C₁₋₆)alkyl, substituted by hydroxy, phenyl,
unsubstituted phenyl and substituted phenyl, wherein the substituents are as defined above for substituted phenyl,
(C₁₋₆)alkoxycarbonylamino, e.g. tert.butoxycarbonylamino,
(C₁₋₆)alkoxycarbonyl(C₁₋₆)alkyl,
(C₃₋₈)cycloalkylcarbonyl(C₁₋₆)alkyl,
(C₃₋₈)cycloalkoxycarbonyl(C₁₋₆)alkyl,
(C₁₋₆)alkylcarbonyl wherein alkyl is unsubstituted or substituted, e.g. by hydroxy,
(C₃₋₈)cycloalkyl,
(C₃₋₈)cycloalkylamino(C₁₋₆)alkyl,

more preferably substituted by (C₁₋₆)alkoxycarbonyl, such as BOC, (C₄₋₈)alkyl, such as pentyl or (C₁₋₆)alkoxycarbonylamino, e.g. tert.butoxycarbonylamino.
R_{18P3} is hydrogen, such as a compound of formula

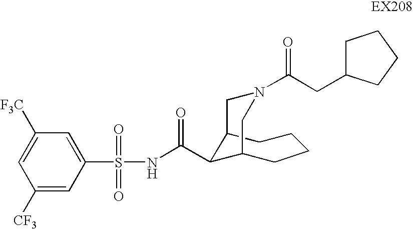

or of formula

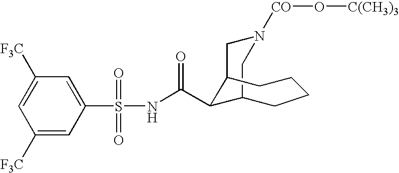

including pure isomers of formula

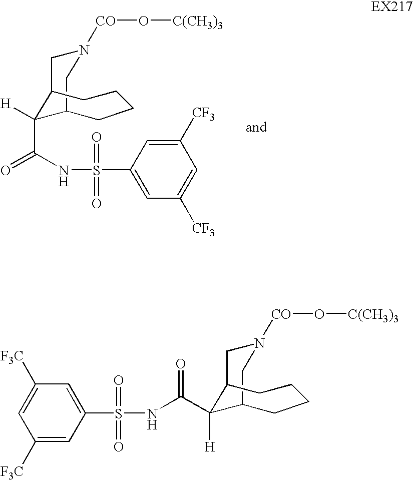

and mixtures thereof.

Compounds comprising a group of formula

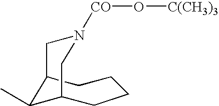

normally are obtained in the configuration of a compound of formula EX217.

A steroid sulfatase inhibitor of the present invention also includes a compound of formula I, which is a compound of formula

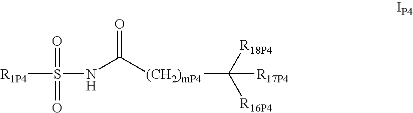

wherein

R_{1P4} has the meaning of R₁ as defined above, R_{16P4} and R_{17P4} together with the carbon atom to which they are attached are a substituted bridged cycloalkyl ring system or substituted piperidine, a substituted bridged heterocyclic system, substituted piperazine, or substituted tetrahydropyridine, wherein the substitutents are as defined above for corresponding groups and wherein piperazine is substituted by groups as defined for substituted piperidine above,

R_{18P4} has the meaning of R₁₈ as defined above, and

m_{P4} is 1, 2, 3 or 4.

In a compound of formula I_{P4} preferably

R_{1P4} is unsubstituted or substituted phenyl or thienyl.

R_{16P4} and R_{17P4} together with the carbon atom to which they are attached are a substituted bridged cycloalkyl ring system, substituted piperidine or substituted bridged piperidine, more preferably a substituted bridged cycloalkyl ring system or substituted piperidine,

wherein substitutents are selected from

a) C₁₋₆)alkoxycarbonyl, e.g. BOC,
(C₁₋₆)alkoxycarbonyl(C₁₋₄)alkyl, e.g. tert.butoxycarbonylmethyl,
(C₁₋₄)alkylcarbonyloxy(C₁₋₄)alkyl, e.g. unsubstituted or substituted by phenyl,
unsubstituted or substituted phenyl, wherein the substituents are as defined above for phenyl,
(C₁₋₆)alkylcarbonyl or phenylcarbonyl,
(C₃₋₈)cycloalkyl(C₁₋₄)alkylcarbonyl,
heterocyclyl, e.g. pyridine, such as pyridin-2-yl, e.g. substituted by nitro, and optionally

b) (C₁₋₄)alkyl at a carbon atom of a ring,

more preferably substitutents are selected from (C₁₋₆)alkoxycarbonyl, e.g. BOC, phenyl, unsubstituted phenyl and substituted phenyl, e.g. substituted by groups as defined above for substituted phenyls, such as nitro, (C₁₋₄)alkyl, (C₁₋₄)haloalkyl, e.g. trifluoromethyl, aminocarbonyl.
R_{18P4} is hydrogen or hydroxy, more preferably hydrogen.
m_{P4} is 1, such as compounds of formula

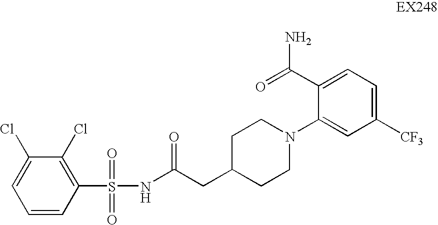

or of formula

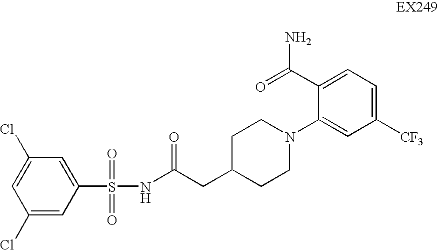

or of formula

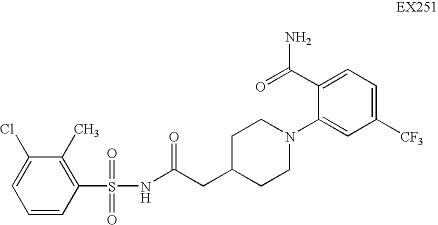

A steroid sulfatase inhibitor of the present invention also includes a compound of formula I, which is a compound of formula

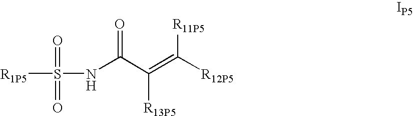

wherein

R_{1P5} has the meaning of R₁ as defined above,

R_{13P5} has the meaning of R₁₃ as defined above, and

R_{11P5} and R_{12P5} together with the carbon atom to which they are attached have the meaning of R₁₁ and R₁₂ as defined above.

In a compound of formula I_{P5} preferably
R_{1P5} is unsubstituted or substituted phenyl or thienyl
R_{11P5} and R_{12P5} together with the carbon atom to which they are attached are piperidine, methylpiperidine or a bridged cycloalkyl ring system substituted by
   (C₁₋₆)alkoxycarbonyl, e.g. tert.butoxycarbonyl;
   unsubstituted or substituted phenyl, wherein the substituents are as defined above for phenyl,
   (C₁₋₈)alkylcarbonyloxy, such as tert.butyl-methylcarbonyloxy,
more preferably substitutents are selected from (C₁₋₈)alkoxycarbonyl, such as BOC, or (C₁₋₆)alkyl-carbonyloxy, such as tert.butylmethylcarbonyloxy,
R_{3P5} is hydrogen, halogen or cyano.

A steroid sulfatase inhibitor of the present invention also includes a compound of formula I, which is a compound of formula

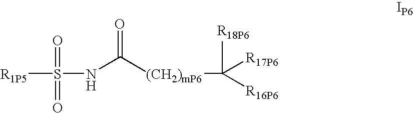

wherein

R_{1P6} has the meaning of R₁ as defined above,

R_{16P6} and R_{17P6} together with the carbon atom to which they are attached are substituted (C₄₋₈)cycloalkyl,

R_{18P6} has the meaning of R₁₈ as defined above, and

m_{P6} is 1, 2, 3 or 4.

In a compound of formula I_{P6} preferably
R_{1P6} is unsubstituted or substituted phenyl or thienyl.
R_{16P6} and R_{17P6} together with the carbon atom to which they are attached are cyclohexyl, substituted by (C₁₋₆)alkoxycarbonyloxy or (C₁₋₆)alkoxycarbonylamino
m_{P6} is 1.

A steroid sulfatase inhibitor of the present invention also includes a compound of formula I, which is a compound of formula

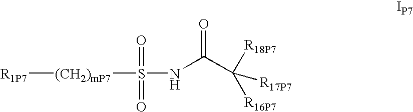

wherein

R_{1P7} has the meaning of R₁ as defined above,

R_{16P7} and R_{17P7} together with the carbon atom to which they are attached are substituted (C₄₋₈)cycloalkyl, wherein the substituents are as defined above for substituted (C₄₋₈)cycloalkyl with the exception of phenyl or substituted phenyl as a substituent,

R_{18P7} has the meaning of R₁₈ as defined above, and

m_{P7} is 1, 2, 3 or 4.

In a compound of formula I_{P7} preferably
R_{1P7} is unsubstituted or substituted phenyl,
R_{16P7} and R_{17P7} together with the carbon atom to which they are attached are cyclohexyl substituted by (C₁₋₆)alkoxycarbonylamino(C₁₋₄)alkyl, or (C₁₋₆)alkoxycarbonylamino, wherein the amine group is optionally further substituted by (C₁₋₄)alkyl.
R_{18P7} is hydrogen, and
m_{P7} is 1.

A steroid sulfatase inhibitor of the present invention also includes a compound of formula I, which is a compound of formula

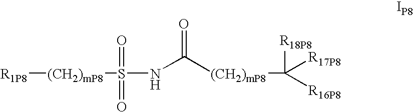

wherein

R_{1P8} has the meaning of R₁ as defined above,

R_{16P8} and R_{17P8} together with the carbon atom to which they are attached are substituted piperidine, tetrahydropyridine or piperazine, wherein the substitutents are as defined above for piperidine,

R_{18P8} has the meaning of R₁₈ as defined above,

m_{P8} is 1 and n_{P8} is 1,

In a compound of formula I_{P8} preferably
R_{1P8} is unsubstituted or substituted phenyl,
R_{16P8} and R_{17P8} together with the carbon atom to which they are attached are piperidine substituted by (C₁₋₆)alkoxycarbonyl.
R_{18P8} is hydrogen.
m_{P8} is 1.
n_{P8} is 1.

A steroid sulfatase inhibitor of the present invention also includes a compound of formula I, which is a compound of formula

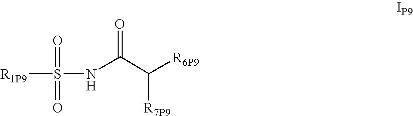

wherein R_{1P9}, R_{6P9} and R_{7P9} have the index-number corresponding meaning of R₁, R₆ and R₇ as defined above and wherein at least one substituent selected from the group consisting of a substituted bridged cycloalkyl ring system, substituted (C₄₋₈)cycloalkyl, substituted piperidine, substituted tetrahydropyridine, substituted piperazine, or a substituted bridged heterocyclyl ring system, wherein the substituents are as defined above for the corresponding groups, is present.

In a compound of formula I_{P9} preferably
R_{1P9} is unsubstituted or substituted phenyl,
R_{6P9} and R_{7P9} independently of each other are (C₁₋₆)haloalkyl, unsubstituted or substituted phenyl, piperidinyl substituted by (C₃₋₈)cycloalkylaminocarbonyl or (C₁₋₆)alkoxycarbonyl, or amino substituted by substituted piperidine,

and wherein at least one substituent is such substituted piperidinyl.

A steroid sulfatase inhibitor of the present invention also includes a compound of formula I, which is a compound of formula

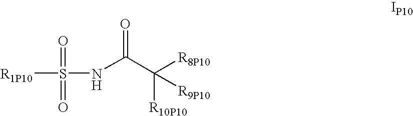

wherein

wherein R_{1P10} has the meaning of R₁,

R_{8P10} is a substituted
bridged cycloalkyl system, (C₄₋₈)cycloalkyl, substituted piperidine, tetrahydropyridine, or a bridged heterocyclic system,

wherein the substitutents are as defined above for the corresponding groups, and

R_{9P10} and R_{10P10} together with the carbon atom to which they are attached are (C₄₋₈)cycloalkyl.

In a compound of formula I_{P10} preferably
R_{1P10} is substituted or unsubstituted phenyl.
R_{8P10} is piperidine substituted by (C₁₋₆)alkoxycarbonyl or unsubstituted or substituted phenyl.
R_{9P10} and R_{10P10} together with the carbon atom to which they are attached are (C₄₋₇)cycloalkyl.

A steroid sulfatase inhibitor of the present invention also includes a compound of formula I, which is a compound of formula

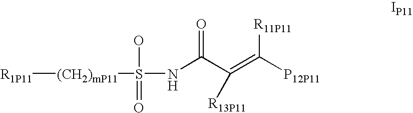

wherein

R_{1P11} has the meaning of R₁,

R_{11P11} and R_{12P11} together with the carbon atom to which they are attached have the meaning of R₁₁ and R₁₂ together with the carbon atom to which they are attached,

R_{13P11} has the meaning of R₁₃, and

m_{P11} is 1, 2, 3 or 4.

In a compound of formula I_{P11} preferably
R_{1P11} is substituted or unsubstituted phenyl.
R_{11P11} and R_{12P11} together with the carbon atom to which they are attached are a substituted bridged cycloalkyl ring system.
m_{P11} is 1.

A steroid sulfatase inhibitor of the present invention also includes a compound of formula I, which is a compound of formula

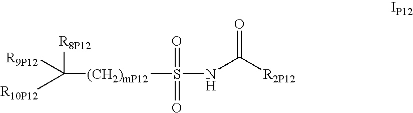

wherein

R_{2P12} has the meaning of R₈ as defined above and additionally is unsubstituted or substituted (C₆₋₁₈)aryl wherein substituents are as defined above for aryl-substituents,

R_{8P12} has the meaning of R₈ as defined above,

R_{9P12} and R_{10P12} have the meaning of R₉ and R₁₀ as defined above, and

m_{P12} is 1, 2, 3 or 4.

In a compound of formula I_{P12} preferably
R_{2P12} is substituted or unsubstituted phenyl.
R_{8P12} is hydrogen or hydroxy.
R_{9P12} and R_{10P12} together with the carbon atom to which they are attached are
A) piperidine substituted at the nitrogen atom of the ring by (C₁₋₆)alkoxycarbonyl, (C₃₋₈)cycloalkyl(C₁₋₄)alkylcarbonyl, or unsubstituted or substituted phenyl,
B) a bridged cycloalkyl ring system substituted by oxo, e.g. and (C₁₋₄)alkyl.
m_{P12} is 1, such as a compound of formula

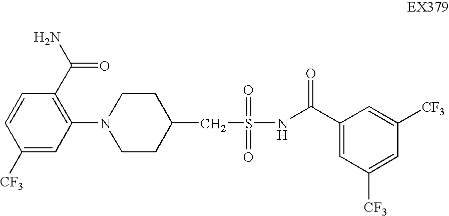

A steroid sulfatase inhibitor of the present invention also includes a compound of formula I, which is a compound of formula

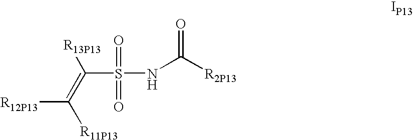

wherein

R_{2P13} has the meaning of R₂ as defined above, and additionally is unsubstituted or substituted (C₆₋₁₈)aryl wherein substituents are as defined above for aryl-substituents,

R_{11P13} and R_{12P13} have the meaning of R₁₁ and R₁₂ as defined above, and

R_{13P13} has the meaning of R₁₃ as defined above.

In a compound of formula I_{P13} preferably
R_{2P13} is unsubstituted or substituted phenyl.
R_{11P13} and R_{12P13} together with the carbon atom to which they are attached are piperidine substituted by unsubstituted or substituted phenyl, or substituted by (C₁₋₆)alkoxycarbonyl.
R_{13P13} is hydrogen.

A steroid sulfatase inhibitor of the present invention also includes a compound of formula I, which is a compound of formula

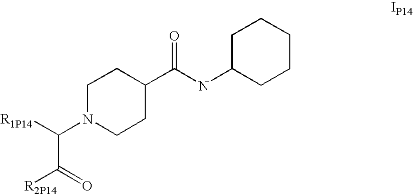

wherein R_{1P14} is (C₆₋₁₈)aryl, and R_{2P14} is (C₆₋₁₈)arylsulfondioxideamino.

In a compound of formula I_{P14} preferably
R_{1P14} is phenyl substituted by trifluoromethyl or halogen, and
R_{2P14} is (C₃₋₁₈)arylsulfondioxideamino, such as phenylsulfondioxideamino, unsubstituted or substituted by (C₁₋₆)alkyl, or halogen(C₁₋₃)alkyl, (C₁₋₃)alkoxy, halogen(C₁₋₃)alkoxy, or halogen.

A compound of formula I includes a compound of formula I_{P1}, I_{P2}, I_{P3}, I_{P4}, I_{P5}, I_{P6}, I_{P7}, I_{P8}, I_{P9}, I_{P10}, I_{P11}, I_{P12}, I_{P13} and I_{P14}. Steroid sulfatase inhibitors include a compound in any form, e.g. in free form, in the form of a salt, in the form of a solvate and in the form of a salt and a solvate. In a steroid sulfatase inhibitor of the present invention substituents indicated are unsubstituted, if not otherwise (specifically) defined. Each single substituent defined above in a compound of formula I may be per se a preferred substituent, independently of the other substituents defined.

A salt of a steroid sulfatase inhibitor of the present invention includes a pharmaceutically acceptable salt, e.g. including a metal salt, an acid addition salt or an amine salt. Metal salts include for example alkali or earth alkali salts; acid addition salts include salts of a compound of formula I with an acid, e.g. HCl; amine salts include salts of a compound of formula I with an amine.

A steroid sulfatase inhibitor of the present invention in free form may be converted into a corresponding compound in the form of a salt; and vice versa. A steroid sulfatase inhibitor of the present invention in free form or in the form of a salt and in the form of a solvate may be converted into a corresponding compound in free form or in the form of a salt in unsolvated form; and vice versa.

Such steroid sulfatase inhibitors may exist in the form of isomers and mixtures thereof, e.g. such compounds may contain asymmetric carbon atoms and may thus exist in the form of diastereoisomeres and mixtures thereof. Substituents in a non-aromatic ring may be in the cis or in the trans configuration in respect to each other. E.g. if R₁ or R₂ includes a substituted piperidine or tetrahydropyridine which is additionally substituted by a further substitutent at a carbon atom of said ring, said further substitutent may be in the cis or in the trans configuration with respect to the (optionally —(CH₂)ₘ— or —(CH₂)ₙ)sulfonamide group also attached to said piperidine or tetrahydropyridine; and if R₁ or R₂ includes a substituted cyclohexyl, said substitutent may be in the cis or in trans configuration with respect to the (optionally —(CH₂)ₘ— or —(CH₂)ₙ)sulfonamide group also attached to said cyclohexyl ring. Isomeric mixtures may be separated as appropriate, e.g. according to a method as conventional, to obtain pure isomers. Steroid sulfatase inhibitors of the present invention include a compound in any isomeric form and in any isomeric mixture.

Any compound described herein may be prepared as appropriate, e.g. according, e.g. analogously, to a method as conventional, e.g. or as specified herein. A steroid sulfatase inhibitor of the present invention, such as a compound of formula I may e.g. be prepared by reaction of a compound of formula

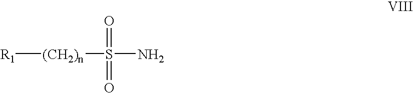

wherein R₁ and n are as defined above, with a compound of formula

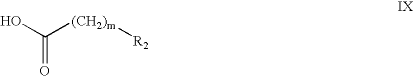

wherein R₂ and m are as defined above, e.g. in an activated form, e.g. and/or in the presence of a coupling agent; and isolating a compound of formula I, wherein R₁, R₂, m and n are as described above from the reaction mixture obtained, e.g. if a compound of formula I comprises a group of formula II or of formula V, a compound of formula VIII may be reacted with a compound of formula

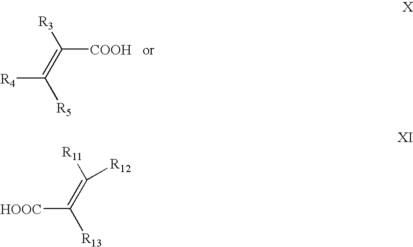

wherein the substituents are as defined above, e.g. in an activated form, e.g. and/or in the presence of a coupling agent, to obtain a compound of formula I, wherein the substitutents are as defined above.

The above reaction is an acylation reaction and may be carried out as appropriate, e.g. in appropriate solvent and at appropriate temperatures, e.g. according, e.g. analogously, to a method as conventional or according, e.g. analogously, to a method as described herein.

If in a compound of formula I a piperidine, tetrahydropyridine or piperazine, or a bridged cycloalkyl ring system comprising a nitrogen atom in a bridge, is unsubstituted present, such ring may be e.g. substituted at the nitrogen atom, e.g. by acylation to introduce a carbonyl containing residue, e.g. or by reaction with a fluoro containing phenyl wherein fluoro acts as a leaving group for N-phenylation (similarly, a heterocyclyl group may be attached to the nitrogen with a corresponding heterocyclic ring which is substituted by chloro as a leaving group). An ester group obtained by a reaction step may be saponified to obtain a carboxylic acid group, or vice versa.

Compounds of formula VIII, IX, X and XI are known or may be obtained as appropriate, e.g. according, e.g. analogously, to a method as conventional or as described herein.

A compound of formula VIII, for example may be obtained from a compound of formula

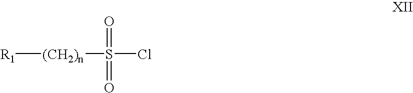

by treatment with (aqueous) NH₃.

A compound of formula X or XI may be obtained e.g. by reacting a compound R₂—H, wherein R₂ is a group of formula II or of formula V, which carries an oxo group at one of the carbon atoms of the (bridged) ring system, with
(RO)₂OP—CHRₓ—COO—R, wherein R is alkyl, such as (C₁₋₄)alkyl, e.g. methyl or ethyl and Rₓ is R₃ or R₈ as defined above, in a solvent, e.g. tetrahydrofurane in the presence of a base e.g. sodium hydride; or
Ph₃-P—CRₓ—COO—C₂H₅, wherein Rₓ is as defined above, in a solvent such as toluene, e.g. at temperatures above room temperature, or,
if Rₓ is hydrogen, by reaction with NC—CH₂—COOR, wherein R is as defined above, in a solvent, e.g. dimethylformamide, in the presence of a catalyst, e.g. piperidine and β-alanine, e.g. at temperatures above room temperature; and subsequent treatment of the compound obtained with NaOH or LiOH, in a solvent such as tetrahydrofurane/H₂O, e.g. at temperatures above room temperature.

Steroidal hormones in particular tissues are associated with several diseases, such as tumors of breast, endometrium and prostate and disorders of the pilosebaceous unit, e.g. acne, androgenetic alopecia, and hirsutism. Important precursors for the local production of these steroid hormones are steroid 3-O-sulfates which are desulfated by the enzyme steroid sulfatase in the target tissues. Inhibition of this enzyme results in reduced local levels of the corresponding active steroidal hormones, which is expected to be of therapeutic relevance. Furthermore, steroid sulfatase inhibitors may be useful as immunosuppressive agents, and have been shown to enhance memory when delivered to the brain.

Acne is a polyetiological disease caused by the interplay of numerous factors, such as inheritance, sebum, hormones, and bacteria. The most important causative factor in acne is sebum production; in almost all acne patients sebaceous glands are larger and more sebum is produced than in persons with healthy skin. The development of the sebaceous gland and the extent of sebum production is controlled hormonally by androgens; therefore, androgens play a crucial role in the pathogenesis of acne. In man, there are two major sources supplying androgens to target tissues: (i) the gonades which secrete testosterone, (ii) the adrenals producing dehydroepiandrosterone (DHEA) which is secreted as the sulfate conjugate (DHEAS). Testosterone and DHEAS are both converted to the most active androgen, dihydrotestosterone (DHT), in the target tissue, e.g. in the skin. There is evidence that these pathways of local synthesis of DHT in the skin are more important than direct supply with active androgens from the circulation. Therefore, reduction of endogeneous levels of androgens in the target tissue by specific inhibitors should be of therapeutic benefit in acne and seborrhea. Furthermore, it opens the perspective to treat these disorders through modulation of local androgen levels by topical treatment, rather than influencing circulating hormone levels by systemic therapies.

Androgenetic male alopecia is very common in the white races, accounting for about 95% of all types of alopecia. Male-pattern baldness is caused by an increased number of hair follicles in the scalp entering the telogen phase and by the telogen phase lasting longer. It is a genetically determined hair loss effected through androgens. Elevated serum DHEA but normal testosterone levels have been reported in balding men compared with non-balding controls, implying that target tissue androgen production is important in androgenetic alopecia.

Hirsutism is the pathological thickening and strengthening of the hair which is characterized by a masculine pattern of hair growth in children and women. Hirsutism is androgen induced, either by increased formation of androgens or by increased sensitivity of the hair follicle to androgens. Therefore, a therapy resulting in reduction of endogeneous levels of androgens and/or estrogens in the target tissue (skin) should be effective in acne, androgenetic alopecia and hirsutism.

As described above, DHT, the most active androgen, is synthesized in the skin from the abundant systemic precursor DHEAS and the first step in the metabolic pathway from DHEAS to DHT is desulfatation of DHEAS by the enzyme steroid sulfatase to produce DHEA. The presence of the enzyme in keratinocytes and in skin-derived fibroblasts has been described. The potential use of steroid sulfatase inhibitors for the reduction of endogenous levels of steroid hormones in the skin was confirmed using known steroid sulfatase inhibitors, such as estrone 3-O-sulfamate and 4-methylumbelliferyl-7-O-sulfamate. We have found that inhibitors of placental steroid sulfatase also inhibit steroid sulfatase prepared from either a human keratinocyte (HaCaT) or a human skin-derived fibroblast cell line (1BR3GN). Such inhibitors were also shown to block steroid sulfatase in intact monolayers of the HaCaT keratinocytes.

Therefore, inhibitors of steroid sulfatase may be used to reduce androgen and estrogen levels in the skin. They can be used as inhibitors of the enzyme steroid sulfatase for the local treatment of androgen-dependent disorders of the pilosebaceous unit (such as acne, seborrhea, androgenetic alopecia, hirsutism) and for the local treatment of squamous cell carcinoma.

Furthermore non-steroidal steroid sulfatase inhibitors are expected to be useful for the treatment of disorders mediated by the action of steroid hormones in which the steroidal products of the sulfatase cleavage play a role. Indications for these new kind of inhibitors include androgen-dependent disorders of the pilosebaceous unit (such as acne, seborrhea, androgenetic alopecia, hirsutism); estrogen- or androgen-dependent tumors, such as squamous cell carcinoma and neoplasms, e.g. of the breast, endometrium, and prostate; inflammatory and autoimmune diseases, such as rheumatoid arthritis, type I and II diabetes, systemic lupus erythematosus, multiple sclerosis, myastenia gravis, thyroiditis, vasculitis, ulcerative colitis, and Crohn's disease, asthma and organ rejection following transplantation, psoriasis, lichen planus, atopic dermatitis, allergic-, irritant-contact dermatitis, eczematous dermatitis, graft versus host disease. Steroid sulfatase inhibitors are also useful for the treatment of cancer, especially for the treatment of estrogen- and androgen-dependent cancers, such as cancer of the breast and endometrium and squamous cell carcinoma, and cancer of the prostata. Steroid sulfatase inhibitors are also useful for the enhancement of cognitive function, especially in the treatment of senile dementia, including Alzheimer's disease, by increasing the DHEAS levels in the central nervous system.

Activities of compounds in inhibiting the activity of steroid sulfatase may be shown in the following test systems:

Purification of Human Steroid Sulfatase

Human placenta is obtained freshly after delivery and stripped of membranes and connective tissues. For storage, the material is frozen at −70° C. After thawing, all further steps are carried out at 4° C., while pH values are adjusted at 20° C. 400 g of the tissue is homogenized in 1.2 I of buffer A (50 mM Tris-HCl, pH 7.4, 0.25 M sucrose). The homogenate obtained is centrifuged at 10,000×g for 45 minutes. The supernatant is set aside and the pellet obtained is re-homogenized in 500 ml of buffer A. After centrifugation, the two supernatants obtained are combined and subjected to ultracentrifugation (100,000×g, 1 hour). The pellet obtained is resuspended in buffer A and centrifugation is repeated. The pellet obtained is suspended in 50 ml of 50 mM Tris-HCl, pH 7.4 and stored at −20° C. until further work-up.

After thawing, microsomes are collected by ultracentrifugation (as described above) and are suspended in 50 ml of buffer B (10 mM Tris-HCl, pH 7.0, 1 mM EDTA, 2 mM 2-mercaptoethanol, 1% Triton X-100, 0.1% aprotinin). After 1 hour on ice with gentle agitation, the suspension is centrifuged (100,000×g, 1 hour). The supernatant containing the enzyme activity is collected and the pH is adjusted to 8.0 with 1 M Tris. The solution obtained is applied to a hydroxy apatite column (2.6×20 cm) and equilibrated with buffer B, pH 8.0. The column is washed with buffer B at a flow rate of 2 ml/min. The activity is recovered in the flow-through. The pool is adjusted to pH 7.4 and subjected to chromatography on a concanavalin A sepharose column (1.6×10 cm) equilibrated in buffer C (20 mM Tris-HCl, pH 7.4, 0.1% Triton X-100, 0.5 M NaCl). The column is washed with buffer C, and the bound protein is eluted with 10% methyl mannoside in buffer C. Active fractions are pooled and dialysed against buffer D (20 mM Tris-HCl, pH 8.0, 1 mM EDTA, 0.1% Triton X-100, 10% glycerol (v/v)).

The retentate obtained is applied to a blue sepharose column (0.8×10 cm) equilibrated with buffer D; which column is washed and elution is carried out with a linear gradient of buffer D to 2 M NaCl in buffer D. Active fractions are pooled, concentrated as required (Centricon 10), dialysed against buffer D and stored in aliquots at −20° C.

Assay of Human Steroid Sulfatase

It is known that purified human steroid sulfatase not only is capable to cleave steroid sulfates, but also readily cleaves aryl sulfates such as 4-methylumbelliferyl sulfate which is used in the present test system as an activity indicator. Assay mixtures are prepared by consecutively dispensing the following solutions into the wells of white microtiter plates:
1) 50 μl substrate solution (1.5 mM 4-methylumbelliferyl sulfate in 0.1 M Tris-HCl, pH 7.5)
2) 50 μl test compound dilution in 0.1 M Tris-HCl, pH 7.5, 0.1% Triton X-100 (stock solutions of the test compounds are prepared in DMSO; final concentrations of the solvent in the assay mixture not exceeding 1%)
3) 50 μl enzyme dilution (approximately 12 enzyme units/ml)

We define one enzyme unit as the amount of steroid sulfatase that hydrolyses 1 nmol of 4-methylumbelliferyl sulfate per hour at an initial substrate concentration of 500 μM in 0.1 M Tris-HCl, pH 7.5, 0.1% Triton X-100, at 37° C.

Plates are incubated at 37° C. for 1 hour. Then the reaction is stopped by addition of 100 μl 0.2 M NaOH. Fluorescence intensity is determined in a Titertek Fluoroskan II instrument with λₑₓ=355 nm and λₑₘ=460 nm.

Calculation of Relative IC₅₀ Values

From the fluorescence intensity data (I) obtained at different concentrations (c) of the test compound in the human steroid sulfatase assay as described above, the concentration inhibiting the enzymatic activity by 50% (IC₅₀) is calculated using the equation:

$$I = \frac{{I}_{100}}{1 + {( c / {IC}_{50} )}^{5}}$$

wherein I₁₀₀ is the intensity observed in the absence of inhibitor and s is a slope factor. Estrone sulfamate is used as a reference compound and its IC₅₀ value is determined in parallel to all other test compounds. Relative IC₅₀ values are defined as follows:

$$rel {IC}_{50} = \frac{{IC}_{50} of test compound}{{IC}_{50} of estrone sulfamate}$$

According to our testing and calculation estrone sulfamate shows an IC₅₀ value of approximately 60 nM.

The steroid sulfatase inhibitors of the present invention show activity in that described assay (rel IC₅₀ in the range of 0.0046 to 10).

CHO/STS Assay

CHO cells stably transfected with human steroid sulfatase (CHO/STS) are seeded into microtiter plates. After reaching approximately 90% confluency, they are incubated overnight with graded concentrations of test substances (e.g. compounds of the present invention). They are then fixed with 4% paraformaldehyde for 10 minutes at room temperature and washed 4 times with PBS, before incubation with 100 μl/well 0.5 mM 4-methylumbelliferyl sulfate (MUS), dissolved in 0.1M Tris-HCl, pH 7.5. The enzyme reaction is carried out at 37° C. for 30 minutes. Then 50 μl/well stop solution (1M Tris-HCl, pH 10.4) are added. The enzyme reaction solutions are transferred to white plates (Microfluor, Dynex, Chantilly, Va.) and read in a Fluoroskan II fluorescence microtiter plate reader. Reagent blanks are subtracted from all values. For drug testing, the fluorescence units (FU) are divided by the optical density readings after staining cellular protein with sulforhodamine B (OD₅₅₀), in order to correct for variations in cell number. IC₅₀ values are determined by linear interpolation between two bracketing points. In each assay with inhibitors, estrone 3-O-sulfamate is run as a reference compound, and the IC₅₀ values are normalized to estrone 3-O-sulfamate (relative IC₅₀=IC₅₀ compound/IC₅₀ estrone 3-O-sulfamate).

The steroid sulfatase inhibitors of the present invention show activity in that described assay (rel IC₅₀ in the range of 0.05 to 10).

Assay Using Human Skin Homogenate

Frozen specimens of human cadaver skin (about 100 mg per sample) are minced into small pieces (about 1×1 mm) using sharp scissors. The pieces obtained are suspended in ten volumes (w/w) of buffer (20 mM Tris-HCl, pH 7.5), containing 0.1% Triton X-100. Test compounds (e.g. compounds of the present invention) are added at graded concentrations from stock solutions in ethanol or DMSO. Second, DHEAS as the substrate is added (1 μC/ml [³H]DHEAS, specific activity: about 60 Ci/mmol, and 20 μM unlabeled DHEAS). Samples are incubated for 18 hrs at 37° C. At the end of the incubation period, 50 μl of 1 M Tris, pH 10.4 and 3 ml of toluene are added. A 1-ml aliquot of the organic phase is removed and subjected to liquid scintillation counting. The determined dpm-values in the aliquots are converted to nmol of DHEA cleaved per g of skin per hour.

The steroid sulfatase inhibitors of the present invention show activity in that described assay (IC₅₀ in the range of 0.03 to 10 μM).

The steroid sulfatase inhibitor of the present invention show activity in test systems as defined above. A steroid sulfatase inhibitor of the present invention in salt and/or solvate form exhibits the same order of activity as a compound of the present invention in free and/or non-solvated form.

The steroid sulfatase inhibitor of the present invention are therefore indicated for use as steroid sulfatase inhibitors in the treatment of disorders mediated by the action of steroid sulfatase, e.g. including androgen-dependent disorders of the pilosebaceous unit, such as
acne,
seborrhea,
androgenetic alopecia,
hirsutism;
cancers, such as estrogen and androgen-dependent cancers;
cognitive dysfunctions, such as senile dementia including Alzheimer's disease.

The steroid sulfatase inhibitor of the present invention are preferably used in the treatment of acne, seborrhea, androgenetic alopecia, hirsutism; estrogen, e.g. and androgen-dependent cancers, more preferably in the treatment of acne. Treatment includes therapeutical treatment and prophylaxis.

Preferred compounds of the present invention include a compound of Example 208, a compound of Example 217 and Example 218, a compound of Example 248, a compound of Example 249, a compound of Example 251, and a compound of Example 379. These compounds show in the Human Steroid Sulfatase Assay a rel IC₅₀ in the range of 0.0046 to 0.29, in the CHO/STS Assay a rel IC₅₀ in the range of 0.05 to 0.18, and in the Assay Using Human Skin Homogenate of an IC₅₀ in the range of 0.03 to 0.27 μM and are thus highly active steroid sulfatase inhibitors. Even more preferred is the compound of Example 217 and Example 218, which show in the Assay of Human Steroid Sulfatase a rel IC₅₀ of 0.29, in the CHO/STS Assay a rel IC₅₀ of 0.08 and in the Assay Using Human Skin Homogenate an IC₅₀ of 0.27 μM.

We have now surprisingly found, that a steroid sulfatase inhibitor, e.g. a compound of Example 217 and a compound of Example 218, show anti-inflammatory activity in combination with an ascomycin, e.g. pimecrolimus.

Activity in inflammatory diseases may be e.g. shown in the following test system

Anti-Inflammatory Test System

The test sites on the inner surface of the right external ears of mice, e.g. strain NMRI, (8 per group) are treated with 10 μl of the dissolved test compound or with the vehicle (a 4:4:2 mixture of ethanol/acetone/dimethylacetamide) alone. The test compounds are applied alone or in combination at concentrations shown in the TEST RESULT TABLE. Thirty minutes after the treatment irritant contact dermatitis is elicitated at the treated auricular sites with 10 μl 0.005% tetradecanoylphorbol-13-acetate (TPA).

Skin inflammation is assessed 6 hours after the elicitation by determination of the auricular weights, as a measure of inflammatory swelling. The animals are killed and both ears are cut off and weighed. Inhibitory activity of test compounds is calculated from differences in right and left ears (internal controls) in mice treated with the test compounds compared with animals treated with the vehicle only. Results obtained are as set out in TEST RESULT TABLE below:

TEST RESULT TABLE

compound of example 217
or of example 218

| pimecrolimus | 0 | 0.1 | 0.3 | 1.0 | 3.0 | 10 |
|---|---|---|---|---|---|---|
| 0 | | | | 20 | 36 | 45 |
| 0.1 | 15 | | 18 | 35 | | 49 |
| 0.3 | 29 | | | 52 | | |
| 1.0 | 30 | | | 58 | | 70 |
| 3.0 | 31 | | | | | 54 |
| 10 | 42 | | | | | 60 |

In the TEST RESULT TABLE the concentrations of the compounds (in bold) used are indicated in micromol/litre. The values given in the TEST RESULT TABLE (in regular letters) are the inhibition in % determined according to the ANTI-INFLAMMATORY TEST SYSTEM used.

From the TEST RESULT TABLE it is evident that a combination of a steroid sulfatase inhibitor with an ascomycin is useful as an anti-inflammatory agent.

In another aspect the present invention provides a combination of a steroid sulfatase inhibitor with an ascomycin for use as a pharmaceutical.

In a further aspect the present invention provides the use of a combination of a steroid sulfatase inhibitor with an ascomycin in the preparation of a medicament for the treatment of inflammatory disorders.

In another aspect the present invention provides a method of treating inflammatory disorders comprising administering a therapeutically effective amount of a combination of a steroid sulfatase inhibitor with an ascomycin to a subject in need of such treatment.

Treatment includes treatment and prophylaxis. For such treatment the term “a steroid sulfatase inhibitor” includes one or more steroid sulfatase inhibitors, preferably one; and the term “an ascomycin” includes one or more ascomycins, preferably one.

For such use/treatment the appropriate dosage of the individual compounds and of the combination of the present invention will, of course, vary depending upon, for example, the chemical nature and the pharmakokinetic data of a compound of the present invention employed, the individual host, the mode of administration and the nature and severity of the conditions being treated. However, in general, satisfactory results in larger mammals, for example humans, may be obtained if an ascomycin of the present invention and a steroid sulfatase inhibitor according to the present invention each are administered at a daily dose of from about 0.1 mg/kg to about 100 mg/kg animal body weight, e.g. conveniently administered in divided doses two to four times daily. For most large mammals the total daily dosage is from about 5 mg to about 5000 mg of a steroid sulfatase inhibitor of the present invention and from about 5 mg to about 5000 mg of an ascomycin of the present invention, conveniently administered, for example, in divided doses up to four times a day or in retarded form. Unit dosage forms appropriately comprise, e.g. from about 1.25 mg to about 2000 mg of a steroid sulfatase inhibitor of the present invention, and e.g. from about 1.25 mg to about 2000 mg of an ascomycin of the present invention, e.g. in admixture with at least one pharmaceutically acceptable excipient, e.g. carrier, diluent.

An appropriate mol ratio of an ascomycin of the present invention to a steroid sulfatase inhibitor of the present invention in combination includes a mol ratio of 0.1:100 to 1:0.1, such as 1:100 to 1:0.5.

Steroid sulfatase inhibitors and ascomycins of the present invention may be administered in the form of a pharmaceutically acceptable salt, e.g. an acid addition salt, metal salt, amine salt; or in free form; optionally in the form of a solvate and may be administered in similar manner to known standards for use in inflammatory indications. Steroid sulfatase inhibitors and ascomycins of the present invention may be admixed with conventional, e.g. pharmaceutically acceptable, excipients, such as carriers and diluents and optionally further excipients. Steroid sulfatase inhibitors and ascomycins of the present invention may be administered by any conventional route, for example enterally, e.g. including nasal, buccal, rectal, oral, administration; parenterally, e.g. including intravenous, intramuscular, subcutaneous administration; or topically; e.g. including epicutaneous, intranasal, intratracheal administration; e.g. in form of coated or uncoated tablets, capsules, injectable solutions or suspensions, e.g. in the form of ampoules, vials, in the form of ointments, creams, gels, pastes, inhaler powder, foams, tinctures, lip sticks, drops, sprays, or in the form of suppositories. The concentrations of the active substance in a pharmaceutical composition will of course vary, e.g. depending on the compound used, the treatment desired and the nature of the composition used. In general, satisfactory results may be obtained at concentrations of from about 0.05 to about 5% such as from about 0.1 to about 1% w/w in topical compositions, and by about 1% w/w to about 90% w/w in oral, parenteral or intravenous compositions.

In another aspect the present invention provides a pharmaceutical composition comprising, in association with at least one pharmaceutically acceptable excipient, a pharmaceutically effective amount of at least one steroid sulfatase inhibitor in combination with at least one ascomycin.

Combinations include
fixed combinations, in which two or more pharmaceutically active agents are in the same pharmaceutical composition,
kits, in which two or more pharmaceutically active agents in separate compositions are sold in the same package, e.g. with instruction for co-administration; and
free combinations in which the pharmaceutically active agents are packaged separately, but instruction for simultaneous or sequential administration are given.

Such pharmaceutical compositions may be manufactured according, e.g. analogously to a method as conventional, e.g. by mixing, granulating, coating, dissolving or lyophilizing processes. Pharmaceutically acceptable excipient includes e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

A pharmaceutical composition of the present invention may comprise as active ingredients a steroid sulfatase inhibitor of the present invention and an ascomycin of the present invention alone, or a combination of the present invention and additionally one or more other pharmaceutically active agents. Such further pharmaceutically active agents include e.g. other anti-inflammatory active compounds.

In the following examples all temperatures are given in degree Centigrade and are uncorrected.

The following abbreviations are used:

DIEA diisopropylethylamine

DMA N,N-dimethylacetamide

DMAP N,N-dimethylaminopyridine

DMF N,N-dimethylformamide

DMSO dimethylsulfoxide

EDC 1-ethyl-3-(3′-dimethylaminopropyl)carbodiimide in the form of a hydrochloride

EtAc ethyl acetate

EX Example

HEX n-hexane

c-HEX cyclohexane

m.p.: melting point

PPA propanephosphonic acid anhydride

RT room temperature

TFA trifluoroacetic acid

THF tetrahydrofurane

Procedures

### EXAMPLE A

### 4-(4-Bromo-2,5-dichloro-thiophene-3-sulfonylaminocarbonyl)-piperidine-1-carboxylic acid tert.-butyl ester (compound of Example 1)

### a. 4-Bromo-2,5-dichloro-thiophene-3-sulfonamide

90 ml of an aqueous solution of NH₃ (32%) are added at RT to a solution of 8.88 g of 4-bromo-2,5-dichloro-thiophene-3-sulfonylchloride in 120 ml of EtAc. The mixture obtained is stirred for ca. 15 hours. Two phases obtained are separated, the organic layer is washed with 1 N HCl and H₂O, and dried. Solvent of the organic phase obtained is evaporated.

4-Bromo-2,5-dichloro-thiophene-3-sulfonamide is obtained.

m.p. 113-117°; ¹³C-NMR (CDCl₃): δ=108.287; 125.342; 130.404; 135.716.

### b. 4-(4-Bromo-2,5-dichloro-thiophene-3-sulfonylaminocarbonyl)-piperidine-1-carboxylic acid tert.-butyl ester

60 mg of DMAP, 130 mg of DIEA and 192 mg of EDC are added to a solution of 155 mg of 4-bromo-2,5-dichloro-thiophene-3-sulfonamide and 230 mg of 1-(tert.butyloxycarbonyl)-piperidine-4-carboxylic acid in 8 ml of DMF. The mixture obtained is stirred for ca. 16 hours at ca. 30°, solvent is evaporated and the evaporation residue obtained is treated with EtAc. The mixture obtained is washed with aqueous 1 N HCl, aqueous saturated NaHCO₃ and brine, and dried. Solvent from the organic phase obtained is evaporated and the evaporation residue is subjected to chromatography. 4-(4-Bromo-2,5-dichloro-thiophene-3-sulfonylaminocarbonyl)-piperidine-1-carboxylic acid tert.-butyl ester is obtained and lyophilized from 1,4-dioxane.

### EXAMPLE B

### 4-(3,5-Bis-trifluoromethyl-benzenesulfonylaminocarbonyl)-cis-3-methyl-piperidine-1-carboxylic acid tert.-butyl ester (compound of Example 72) and

### 4-(3,5-Bis-trifluoromethyl-benzenesulfonylaminocarbonyl)-trans-3-methyl-piperidine-1-carboxylic acid tert.-butyl ester (compound of Example 73)

18 ml of a sodium bis(trimethylsilyl)amide solution (2M) in THF are added to a suspension of 12.4 g of methoxymethyltriphenylphosphonium chloride in 25 ml of dry THF at 0°. To the mixture obtained, 5.87 g of 3-methyl-4-oxo-piperidine-1-carboxylic acid tert.butyl ester in 25 ml of THF are slowly added, the mixture obtained is stirred at 0°, diluted with EtAc and extracted with aqueous 1M HCl, saturated aqueous NaHCO₃ solution and brine. The organic layer obtained is dried and solvent is evaporated. The evaporation residue obtained is subjected to filtration over silica gel and solvent of the filtrate obtained is evaporated. 3.6 g of the filtration residue obtained are dissolved in 150 ml of CH₃CN, 1.68 g of cerium trichloride heptahydrate and 337 mg of Nal are added and the resulting mixture is stirred at 40° overnight. From the mixture obtained solvent is evaporated and the evaporation residue obtained is treated with EtAc. The mixture obtained is extracted with aqueous 1M HCl, saturated aqueous NaHCO₃ solution and brine. The organic layer obtained is dried, solvent is evaporated and the evaporation residue obtained is subjected to filtration over silica gel and solvent of the filtrate obtained is evaporated. 494 mg of the evaporation residue obtained and 1.18 g of magnesium monoperoxyphthalic acid hexahydrate in 36 ml of EtOH/H₂O (1:1) are stirred at RT and diluted with EtAc. The mixture obtained is extracted with aqueous 1M HCl. The organic layer obtained is dried, solvent is evaporated and the evaporation residue is subjected to filtration and solvent of the filtrate obtained is evaporated. To a solution of 60 mg of the evaporation residue obtained, 71 mg of 3,5-bis(trifluoromethyl)phenylsulfonamide, 94 mg of EDC and 30 mg of DMAP in 2 ml of DMF and 84 μl of DIEA are added and the mixture obtained is shaked at RT. From the mixture obtained solvent is removed and the concentrated residue obtained is subjected to preparative HPLC on an RP-18 column (CH₃CN/H₂O (0.1% TFA).

4-(3,5-Bis-trifluoromethyl-benzenesulfonylaminocarbonyl)-cis-3-methyl-piperidine-1-carboxylic acid tert.-butyl ester and 4-(3,5-Bis-trifluoromethyl-benzenesulfonyl-aminocarbonyl)-trans-3-methyl-piperidine-1-carboxylic acid tert.-butyl ester are obtained.

### EXAMPLE C

### N-[1-(2-Nitro-phenyl)-piperidine-4-carbonyl]-3,5-bis-trifluoromethyl-benzenesulfonamide (compound of Example 81)

### a. N-(Piperidine-4-carbonyl)-3,5-bis-trifluoromethyl-benzenesulfonamide in the form of a hydrochloride

2 g of 4-(3,5-bis-trifluoromethyl-benzenesulfonylaminocarbonyl)-piperidine-1-carboxylic acid tert.-butyl ester are dissolved in a mixture of 1 ml MeOH and 9 ml of CH₂Cl₂. The mixture obtained is treated at RT with 20 ml of 3 N HCl in (C₂H₅)₂O for ca. 16 hours. Solvent is evaporated and N-(piperidine-4-carbonyl)-3,5-bis-trifluoromethyl-benzenesulfonamide in the form of a hydrochloride is obtained. m.p. 285-291°.

### b. N-[1-(2-Nitro-phenyl)-piperidine-4-carbonyl]-3,5-bis-trifluoromethyl-benzenesulfonamide

0.13 g of DIEA and 0.07 g of 1-fluoro-2-nitrobenzene are added to a solution of 0.22 g N-(piperidine-4-carbonyl)-3,5-bis-trifluoromethyl-benzenesulfonamide in the form of a hydrochloride in 4 ml of DMSO. The mixture obtained is stirred for ca. 18 hours at 80°, solvent is evaporated and the evaporation residue obtained is subjected to flash chromatography on silica gel (eluent: EtAc) N-[1-(2-Nitro-phenyl)-piperidine-4-carbonyl]-3,5-bis-trifluoromethyl-benzenesulfonamide is obtained.

### EXAMPLE D

### trans-[4-(4-Bromo-2,5-dichloro-thiophene-3-sulfonylaminocarbonyl)-cyclohexylmethyl]-carbamic acid tert-butyl ester (compound of Example 109)

### a. 4-Bromo-2,5-dichloro-thiophene-3-sulfonamide

90 ml of an aqueous solution of NH₃ (32%) is added at RT to a solution of 8.88 g of 4-bromo-2,5-dichloro-thiophene-3-sulfonylchloride in 120 ml of EtAc. The mixture obtained is stirred for ca. 15 h and two phases obtained are separated. The organic layer obtained is washed with 1 N HCl and H₂O, and dried. Solvent of the organic solution obtained is evaporated. 4-Bromo-2,5-dichloro-thiophene-3-sulfonamide is obtained.

m.p. 113-117° C., ¹³C-NMR: δ=108.287; 125.342; 130.404; 135.716.

### b. trans-[4-(4-Bromo-2,5-dichloro-thiophene-3-sulfonylaminocarbonyl)-cyclohexylmethyl]-carbamic acid tert.-butyl ester

60 mg of DMAP, 130 mg of DIEA and 192 mg of EDC are added to a solution of 155 mg of 4-bromo-2,5-dichloro-thiophene-3-sulfonamide and 257 mg of trans-1-(tert.butyloxycarbonyl-aminomethyl)cyclohexane-4-carboxylic acid in 8 ml of DMF and the mixture obtained is stirred for ca. 16 hours at ca. 30°. From the mixture obtained solvent is evaporated and the evaporation residue obtained is dissolved in EtAc. The solution obtained is washed with 1 N HCl, saturated NaHCO₃ solution and brine, and dried. From the organic phase obtained solvent is evaporated and the evaporation residue obtained is subjected to chromatography. trans-[4-(4-Bromo-2,5-dichloro-thiophene-3-sulfonylaminocarbonyl)-cyclohexylmethyl]-carbamic acid tert.-butyl ester is obtained.

### EXAMPLE E

### 4-Chloro-N-(4-pentyl-bicyclo[2.2.2]octane-1-carbonyl)-benzenesulfonamide (compound of Example 186)

0.42 g of 4-chlorophenylsulfonamide, 60 mg of DMAP and 0.42 g of EDC are added to a solution of 0.5 g of 4-pentyl-bicyclo[2.2.2]octan-1-carboxylic acid in 8 ml of DMF, the mixture obtained is stirred for ca. 16 hours at RT and solvent from the mixture obtained is evaporated. The evaporation residue obtained is dissolved in EtAc and washed with 1 N HCl, saturated NaHCO₃ solution and brine, and the organic phase obtained is dried. Solvent of the organic phase obtained is evaporated and the evaporation residue obtained is subjected to chromatography.

4-Chloro-N-(4-pentyl-bicyclo[2.2.2]octane-1-carbonyl)-benzenesulfonamide is obtained.

### EXAMPLE F

### 10-(3,5-bis-trifluoromethyl-benzenesulfonylaminocarbonyl)-8-aza-bicyclo[4.3.1]decane-8-carboxylic acid tert-butyl ester (compound of Example 217)

### a. 10-Oxo-8-aza-bicyclo[4.3.1]decane-8-carboxylic acid tert-butyl ester

25 g of 8-methyl-8-aza-bicyclo[4.3.1]decan-10-one in the form of a hydrobromide are dissolved in H₂O and a pH of ˜11 is adjusted by addition of aqueous NaOH solution. The mixture obtained is extracted with (C₂H₅)₂O. The organic layer obtained is dried and solvent is evaporated. The evaporation residue obtained is dissolved in 50 ml of dichloroethane, 23.7 ml of 1-chloroethyl chloroformate are added at 0° and the mixture obtained is stirred at 80°, cooled to RT, and 50 ml of MeOH are added. The mixture obtained is stirred at 60°, solvent is evaporated and the evaporation residue obtained together with 18 g of K₂CO₃ and 28.4 g of di-tert.-butyldicarbonate is treated with 240 ml of THF/H₂O (5:1) and stirred at RT. The mixture obtained is concentrated and diluted with EtAc. The mixture obtained is extracted with H₂O, 1M HCl, aqueous, saturated NaHCO₃ solution and brine. The organic layer obtained is dried and solvent is evaporated. The evaporation residue obtained is subjected to filtration over silica gel with EtAc/c-Hex (1:3).

10-Oxo-8-aza-bicyclo[4.3.1]decane-8-carboxylic acid tert-butyl ester is obtained.

m.p.: 50-52°; ¹³C-NMR: 211.99, 154.82, 80.20, 48.70, 28.44, 26.40.

### b. 10-Methoxymethylene-8-aza-bicyclo[4.3.1]decane-8-carboxylic acid tert-butyl ester

To a suspension of 9.54 g of methoxymethyltriphenylphosphonium chloride in 25 ml of dry THF, 13.8 ml of a sodium bis(trimethylsilyl)amide solution (2M) in THF are added at 0° under stirring. To the mixture obtained 5.40 g of 10-oxo-8-aza-bicyclo[4.3.1]decane-8-carboxylic acid tert-butyl ester in 25 ml of THF are slowly added and stirring at 0° is continued. The mixture obtained—diluted with EtAc—is extracted with aqueous 1M HCl, aqueous saturated NaHCO₃ solution and brine. The organic layer obtained is dried and solvent is evaporated. The evaporation residue obtained is subjected to filtration over silica gel with EtAc/c-Hex (1:9). 10-Methoxymethylene-8-aza-bicyclo[4.3.1]decane-8-carboxylic acid tert-butyl ester is obtained.

¹³C-NMR: 155.54, 142.46, 118.38, 79.58, 59.82, 52.17, 50.89, 49.54, 36.93, 35.53, 34.91, 33.80, 33.50, 32.08, 28.94, 27.30, 27.18.

### c. 10-Formyl-8-aza-bicyclo[4.3.1]decane-8-carboxylic acid tert-butyl ester

4.8 g of 10-methoxymethylene-8-aza-bicyclo[4.3.1]decane-8-carboxylic acid tert-butyl ester are dissolved in 180 ml of CH₃CN, 1.94 g of cerium trichloride heptahydrate and 390 mg of Nal are added and the mixture obtained is stirred at 40° overnight. From the mixture obtained solvent is evaporated and the evaporation residue obtained is dissolved in EtAc. The mixture obtained is extracted with aqueous 1M HCl, aqueous, saturated NaHCO₃ solution and brine. The organic layer obtained is dried, solvent is evaporated and the evaporation residue obtained is subjected to filtration over silica gel with EtAc/c-Hex (1:4→1:2).

10-Formyl-8-aza-bicyclo[4.3.1]decane-8-carboxylic acid tert-butyl ester is obtained.

m.p.: 55-60°; ¹³C-NMR: 204.53, 155.28, 78.00, 55.40, 32.44, 32.12, 30.06, 28.89, 27.29.

### d. 8-Aza-bicyclo[4.3.1]decane-8,10-dicarboxylic acid 8-tert-butyl ester

2.86 g of 10-formyl-8-aza-bicyclo[4.3.1]decane-8-carboxylic acid tert-butyl ester and 5.8 g of magnesium monoperoxyphthalic acid hexahydrate in 170 ml of EtOH/H₂O (1:1) are stirred at RT. The mixture obtained is diluted with EtAc. The mixture obtained is extracted with aqueous 1M HCl and brine. The organic layer obtained is dried, solvent is evaporated and the evaporation residue is crystallized from MeOH/H₂O.

8-aza-bicyclo[4.3.1]decane-8,10-dicarboxylic acid 8-tert-butyl ester is obtained. m.p.: 218-222°; ¹³C-NMR: 179.88, 155.31, 80.00, 52.43, 50.98, 47.63, 33.14, 32.31, 28.91, 27.06.

### e. 10-(3,5-Bis-trifluoromethyl-benzenesulfonylaminocarbonyl)-8-aza-bicyclo[4.3.1]decane-8-carboxylic acid tert-butyl ester

6.1 ml of a 50% PPA solution in DMF, 633 mg of DMAP in 50 ml of dimethylamine and 1.8 ml of DIEA are added to a solution of 1.5 g of 8-aza-bicyclo[4.3.1]decane-8,10-dicarboxylic acid 8-tert-butyl ester, 2.3 g of 3,5-bis(trifluoromethyl)phenylsulfonamide, the mixture obtained is stirred at 40° and diluted with EtAc. The mixture obtained is extracted with aqueous 1M NaHSO₄ solution, saturated NaHCO₃ solution and brine. From the mixture obtained solvent is distilled off. The distillation residue obtained is purified by filtration over silica gel with EtAc/c-Hex/MeOH (5:5:1) and the residue obtained is subjected to crystallization from CH₃CN:H₂O (4:6). 10-(3,5-Bis-trifluoromethylbenzenesulfonylamino-carbonyl)-8-aza-bicyclo[4.3.1]decane-8-carboxylic acid tert-butyl ester in the form of a sodium salt is obtained which is dissolved in EtAc and 1 M aqueous HCl and H₂O, the phases obtained are separated, the organic layer obtained is dried and solvent is evaporated. 10-(3,5-bis-trifluoromethyl-benzene-sulfonylaminocarbonyl)-8-aza-bicyclo[4.3.1]decane-8-carboxylic acid tert-butyl ester is obtained.

### EXAMPLE G

### 2-{4-[2-(3,5-Bis-trifluoromethyl-benzenesulfonylamino)-2-oxo-ethyl]-piperidin-1-yl}-4-trifluoromethyl-benzamide (compound of Example 241)

### a. 3,5-Bis-(trifluoromethyl)benzene-sulfonamide

An aqueous solution of NH₃ (32%) is added at RT to a solution of 3,5-bis(trifluoromethyl)-benzene-sulfonylchloride in EtAc. The mixture obtained is stirred and two phases are obtained and are separated. The organic layer obtained is washed with 1 N HCl and H₂O, and dried. Solvent of the organic solution obtained is evaporated.

3,5-Bis-trifluoromethyl-benzene sulfonamide is obtained.

### b. 2-{4-[2-(3,5-Bis-trifluoromethyl-benzenesulfonylamino)-2-oxo-ethyl]-piperidin-1-yl}-4-trifluoromethyl-benzamide

0.46 g of 2-fluoro-4-(trifluoromethyl)benzamide are added to a suspension of 1.8 g K₂CO₃ and 0.8 g of piperidin-4-yl acetic acid hydrochloride in 12 ml of DMSO, the mixture obtained is stirred for 4 hours at 150°, solvent is evaporated, the evaporation residue obtained is suspended in MeOH and filtrated. The filtrate obtained is concentrated and subjected to chromatography on silica gel. [1-(2-Carbamoyl-5-trifluoromethyl-phenyl)-piperidin-4-yl]-acetic acid is obtained. 300 mg of EDC are added to a solution of 260 mg of [1-(2-carbamoyl-5-trifluoromethyl-phenyl)-piperidin-4-yl]-acetic acid, 230 mg of 3,5-bis-trifluoromethyl-benzenesulfonamide, 200 mg of DIEA and 90 mg of DMAP in 4 ml of DMF. The mixture obtained is stirred for 3 days at RT, solvent is evaporated and the evaporation residue obtained is treated with EtAc. The mixture obtained is washed with 1 N HCl, saturated aqueous NaHCO₃ solution and brine, dried, concentrated and subjected to chromatography on silica gel. 2-{4-[2-(3,5-Bis-trifluoromethyl-benzenesulfonylamino)-2-oxo-ethyl]-piperidin-1-yl}-4-trifluoromethyl-benzamide is obtained.

### EXAMPLE H

### 3-[2-(4-Bromo-2,5-dichloro-thiophene-3-sulfonylamino)-2-oxo-ethyl]-9-aza-bicyclo[3.3.1]nonane-9-carboxylic acid tert-butyl ester (compound of Example 242)

### a. 3-Oxo-9-aza-bicyclo[3.3.1]nonane-9-carboxylic acid tert-butyl ester

19.1 g of 9-methyl-9-aza-bicyclo[3.3.1]nonan-3-one in the form of a hydrochloride are suspended in 150 ml of dichloroethane and 26 ml of DIEA are added slowly at 0°. The mixture obtained is stirred for 1 hour at 0°, to the mixture obtained 27 ml of 1-chloroethyl chloroformate are added and the mixture obtained is stirred at 80° for 8 hours and cooled to RT. To the mixture obtained 100 ml of MeOH are added, the mixture obtained is stirred at 60° for 5 hours and solvent is evaporated. To the evaporation residue obtained, 18 g of K₂CO₃ and 28.4 g of di-tert.-butyldicarbonate are added and treated with 250 ml of THF/H₂O, the mixture obtained is stirred at RT for 3 hours, concentrated and diluted with EtAc. The mixture obtained is washed with H₂O, 1M HCl, saturated NaHCO₃ solution and brine, the organic layer obtained is dried and solvent is evaporated. The evaporation residue obtained is subjected to filtration over silica gel.

3-Oxo-9-aza-bicyclo[3.3.1]nonane-9-carboxylic acid tert-butyl ester is obtained. ¹³C-NMR: 209.94, 168.09, 154.33, 80.56, 48.90, 47.58, 45.81, 45.61, 30.95, 30.67, 28.81, 16.67.

### b. 3-Ethoxycarbonylmethylene-9-aza-bicyclo[3.3.1]nonane-9-carboxylic acid tert-butyl ester

0.54 ml of (diethoxy-phosphoryl)-acetic acid ethyl ester are added dropwise to a suspension of 108 mg of NaH (55% in mineral oil) in 5 ml of THF at 0°. The mixture obtained is stirred and 650 mg of 3-oxo-9-aza-bicyclo[3.3.1]nonane-9-carboxylic acid tert-butyl ester in 5 ml of THF are slowly added. The mixture obtained is stirred at 60° for 3 days, diluted with c-HEX and washed with 1M aqueous NaH₂PO₄ and saturated aqueous NaHCO₃ solution. The organic layer obtained is dried, solvent is evaporated and the evaporation residue obtained is subjected to chromatography on silica gel. 3-Ethoxycarbonylmethylene-9-aza-bicyclo[3.3.1]nonane-9-carboxylic acid tert-butyl ester is obtained. ¹³C-NMR: 171.79, 154.45, 154.27, 133.38, 132.77, 127.11, 126.30, 79.64, 79.54, 61.03, 61.00, 48.59, 47.20, 46.81, 45.22, 42.72, 33.61, 33.42, 32.59, 32.17, 30.73, 30.07, 28.87, 28.57, 28.13, 16.48, 14.59.

### c. 3-Ethoxycarbonylmethyl-9-aza-bicyclo[3.3.1]nonane-9-carboxylic acid tert-butyl ester

390 mg of 3-ethoxycarbonylmethylene-9-aza-bicyclo[3.3.1]nonane-9-carboxylic acid tert-butyl ester are dissolved in 50 ml of EtOH and hydrogenated (50 bar, RT) in the presence of 100 mg of PtO₂ as a catalyst. From the mixture obtained the catalyst is filtrated off and 3-ethoxycarbonylmethyl-9-aza-bicyclo[3.3.1]nonane-9-carboxylic acid tert-butyl ester in the form of a mixture of the syn and anti isomers is obtained. ¹³C-NMR: 172.95, 172.88, 155.55, 154.44, 79.46, 79.42, 60.63, 47.40, 45.96, 45.88, 44.60, 43.77, 40.69, 37.01, 36.63, 32.24, 32.03, 31.40, 31.02, 29.61, 29.21, 29.17, 27.43, 20.60, 14.65, 14.07.

### d. 3-Carboxymethyl-9-aza-bicyclo[3.3.1]nonane-9-carboxylic acid tert-butyl ester

10 ml of 1M aqueous NaOH are added to a solution of 3-ethoxycarbonylmethyl-9-aza-bicyclo[3.3.1]nonane-9-carboxylic acid tert-butyl ester in 20 ml of THF and the mixture obtained is stirred at RT. To the mixture obtained 10 ml of brine and 70 ml of EtAc are added, and the mixture obtained is washed with 1M aqueous HCl. The organic layer obtained is dried and solvent is evaporated.

3-Carboxymethyl-9-aza-bicyclo[3.3.1]nonane-9-carboxylic acid tert-butyl ester is obtained.

¹³C-NMR: 178.47, 177.28, 155.61, 154.50, 79.70, 79.63, 47.39, 45.88, 43.39, 40.31, 36.92, 32.22, 31.98, 31.37, 30.99, 30.74, 30.64, 30.08, 29.59, 29.20, 21.15, 20.60, 14.05.

### e. 3-[2-(4-Bromo-2,5-dichloro-thiophene-3-sulfonylamino)-2-oxo-ethyl]-9-aza-bicyclo[3.3.1]nonane-9-carboxylic acid tert-butyl ester

69 μl of DIEA are added to a solution of 57 mg of 3-carboxymethyl-9-aza-bicyclo[3.3.1]nonane-9-carboxylic acid tert-butyl ester, 93 mg of 2,4,5-trichloro-thiophene-3-sulfonic acid amide, 233 μl of PPA and 24 mg of DMAP in 2 ml of DMA, and the mixture obtained is stirred at RT for 48 hours. From the mixture obtained solvent is evaporated and the evaporation residue obtained is subjected to preparative HPLC on an RP-18 column followed by lyophilisation from dioxane.

3-[2-(4-Bromo-2,5-dichloro-thiophene-3-sulfonylamino)-2-oxo-ethyl]-9-aza-bicyclo[3.3.1]nonane-9-carboxylic acid tert-butyl ester is obtained.

### EXAMPLE J

### 9-[1-Fluoro-2-oxo-2-(2,4,5-trichloro-thiophene-3-sulfonylamino)-ethylidene]-3-aza-bicyclo[3.3.1]nonane-3-carboxylic acid tert-butyl ester (compound of Example 288)

### a. 9-Oxo-3-aza-bicyclo[3.3.1]decane-3-carboxylic acid tert-butyl ester

20 g of 3-methyl-3-aza-bicyclo[3.3.1]decan-10-one oxalate are dissolved in H₂O and the pH is adjusted to ˜11 by addition of 1M aqueous NaOH solution. The mixture obtained is extracted with (C₂H₅)₂O, the organic layer obtained is dried and solvent is evaporated. The evaporation residue obtained is dissolved in 100 ml of dichloroethane, 22.5 ml of 1-chloroethyl chloroformate are added at 0°, the mixture obtained is stirred at 80°, cooled to RT and 100 ml of MeOH are added. The mixture obtained is stirred at 60° and solvent is evaporated. The evaporation residue obtained, 14.8 g of K₂CO₃ and 23.4 g of di-tert.-butyldicarbonate are treated with 300 ml of THF/H₂O and stirred at RT. The mixture obtained is concentrated, diluted with EtAc and washed with H₂O, 1M HCl, saturated aqueous NaHCO₃ solution and brine. The organic layer obtained is dried, solvent is evaporated and the evaporation residue is subjected to filtration over silica gel with EtAc/c-HEX.

9-Oxo-3-aza-bicyclo[3.3.1]decane-3-carboxylic acid tert-butyl ester is obtained.

¹³C-NMR: 216.58, 154.49, 80.36, 51.00, 50.15, 47.11, 34.08, 28.45, 19.49.

### b. 9-(Fluoro-Ethoxycarbonylmethylene-3-aza-bicyclo[3.3.1]nonane-3-carboxylic acid tert-butyl ester

1.14 ml of (diethoxy-phosphoryl)-fluoro-acetic acid ethyl ester are added dropwise to a suspension of 244 mg of NaH (55% in mineral oil) in THF at 0°, the mixture obtained is stirred, 918 mg of 9-oxo-3-aza-bicyclo[3.3.1]decane-3-carboxylic acid tert-butyl ester in 10 ml of THF are added slowly and the mixture obtained is stirred at RT overnight. The mixture obtained is diluted with c-HEX and the diluted mixture obtained is washed with 1M aqueous NaH₂PO₄ and saturated aqueous NaHCO₃ solution. The organic layer obtained is dried, solvent is removed by distillation and the distillation residue obtained is subjected to chromatography on silica gel. 9-(Fluoro-ethoxycarbonylmethylene-3-aza-bicyclo[3.3.1]-nonane-3-carboxylic acid tert-butyl ester is obtained.

¹³C-NMR: 161.43, 161.15, 154.65, 139.95, 139.4, 137.97, 79.79, 61.15, 50.33, 49.98, 48.97, 48.53, 31.39, 31.04, 30.98, 28.54, 28.49, 19.70, 14.14.

### c. 9-(Carboxy-fluoro-methylene)-3-aza-bicyclo[3.3.1]nonane-3-carboxylic acid tert-butyl ester

10 ml of 1M aqueous NaOH are added to a solution of 9-(fluoro-ethoxycarbonylmethylene-3-aza-bicyclo[3.3.1]nonane-3-carboxylic acid tert-butyl ester in 20 ml of THF, the mixture obtained is stirred at 40°, 10 ml of brine are added and the mixture obtained is diluted with EtAc. The diluted mixture obtained is washed with 1M aqueous HCl, the organic layer obtained is dried and solvent is evaporated. 9-(Carboxy-fluoro-methylene)-3-aza-bicyclo[3.3.1]nonane-3-carboxylic acid tert-butyl ester is obtained.

¹³C-NMR: 165.25, 164.96, 154.81, 142.21, 139.37, 137.42, 80.23, 50.39, 50.03, 49.37, 49.05, 33.21, 33.10, 32.94, 32.81, 31.74, 31.73, 31.37, 31.31, 28.51, 19.64.

### d. 9-[1-Fluoro-2-oxo-2-(2,4,5-trichloro-thiophene-3-sulfonylamino)-ethylidene]-3-aza-bicyclo[3.3.1]nonane-3-carboxylic acid tert-butyl ester

69 μl of DIEA are added to a solution of 60 mg of 9-(carboxy-fluoro-methylene)-3-aza-bicyclo[3.3.1]nonane-3-carboxylic acid tert-butyl ester, 71 mg of 2,4,5-trichloro-thiophene-3-sulfonyl amide, 233 μl of PPA and 24 mg of DMAP in 2 ml of DMA, and the mixture obtained is stirred at 40° overnight. The mixture obtained is diluted with 10 ml of EtAc/c-HEX, and washed with 1M NaHSO₄ solution. The organic layer obtained is dried and solvent is evaporated. The evaporation residue obtained is subjected to chromatography on silica gel and on Sephadex LH20 (MeOH) and relevant fractions obtained from chromatography are subjected to lyophilisation from dioxane.

9-[1-Fluoro-2-oxo-2-(2,4,5-trichloro-thiophene-3-sulfonylamino)-ethylidene]-3-aza-bicyclo[3.3.1]nonane-3-carboxylic acid tert.-butyl ester is obtained.

### EXAMPLE K

### 3-[2-(4-Bromo-2,5-dichloro-thiophene-3-sulfonylamino)-1-cyano-2-oxo-ethylidene]-8-aza-bicyclo[3.2.1]octane-8-carboxylic acid tert-butyl ester (compound of Example 289)

### a. 3-(Cyano-methoxycarbonyl-methylene)-8-aza-bicyclo[3.2.1]octane-8-carboxylic acid tert-butyl ester

A solution of 2 g of 3-oxo-8-aza-bicyclo[3.2.1]octane-8-carboxylic acid tert-butyl ester, 1.2 ml of cyano-acetic acid methyl ester, 130 μl of piperidine and 38 mg of β-alanine in 4 ml of DMF is stirred at 70° C. for 48 hours, the mixture obtained is diluted with EtAc, washed with H₂O and brine, the organic layer obtained is dried, solvent is evaporated and the residue obtained is subjected to chromatography on silica gel. 3-(cyano-methoxycarbonyl-methylene)-8-aza-bicyclo[3.2.1]octane-8-carboxylic acid tert-butyl ester is obtained

¹³C-NMR: 174.13, 162.27, 153.68, 115.37, 107.45, 80.70, 53.92, 53.08, 28.81.

### b. 3-(Carboxy-cyano-methylene)-8-aza-bicyclo[3.2.1]octane-8-carboxylic acid tert-butyl ester

3-(cyano-methoxycarbonyl-methylene)-8-aza-bicyclo[3.2.1]octane-8-carboxylic acid tert-butyl ester is saponified analogously to the method described in example J, c). 3-(Carboxy-cyano-methylene)-8-aza-bicyclo[3.2.1]octane-8-carboxylic acid tert-butyl ester is obtained.

¹³C-NMR: 165.14, 153.83, 115.12, 107.51, 81.23, 28.82.

### c. 3-[2-(4-Bromo-2,5-dichloro-thiophene-3-sulfonylamino)-1-cyano-2-oxo-ethylidene]-8-aza-bicyclo[3.2.1]octane-8-carboxylic acid tert-butyl ester

120 μl of DIEA are added to a solution of 102 mg of 3-(carboxy-cyano-methylene)-8-aza-bicyclo[3.2.1]octane-8-carboxylic acid tert-butyl ester, 162 mg of 4-bromo-2,5-dichloro-thiophene-3-sulfonamide, 583 μl of PPA in DMF (50%) and 43 mg of DMAP in 4 ml of DMA, and the mixture obtained is stirred at RT for 48 hours. From the mixture obtained solvent is evaporated and the residue obtained is subjected to preparative HPLC on an RP-18 column. 3-[2-(4-Bromo-2,5-dichloro-thiophene-3-sulfonylamino)-1-cyano-2-oxo-ethylidene]-8-aza-bicyclo[3.2.1]octane-8-carboxylic acid tert-butyl ester is obtained.

### EXAMPLE L

### 3,3-Dimethyl-butyric acid 4-[2-(4-bromo-2,5-dichloro-thiophene-3-sulfonylamino)-1-fluoro-2-oxo-ethylidene]-adamantan-1-yl ester (compound of Example 290)

### a. 3,3-Dimethyl-butyric acid 4-oxo-adamantan-1-yl ester

A solution of 1.03 g of 5-hydroxy-2-adamantanone, 1.83 g of DMAP and 1.9 ml of 3,3-dimethylbutanoyl chloride in 10 ml of CH₂Cl₂ is stirred at 40° C. for 48 hours, 6 ml of aqueous 1M KH₂PO₄ solution are added and the mixture obtained is stirred. The layers obtained are separated, from the organic layer obtained solvent is evaporated and the evaporation residue obtained is subjected to chromatography.

3,3-Dimethyl-butyric acid 4-oxo-adamantan-1-yl ester is obtained.

¹³C-NMR: 215.61, 171.52, 49.10, 47.02, 41.38, 39.93, 38.17, 30.74, 29.79, 29.62.

### b. 3,3-Dimethyl-butyric acid 4-(fluoro-ethoxycarbonyl-methylene)-adamantan-1-yl ester

1.48 ml of (diethoxy-phosphoryl)-fluoro-acetic acid ethyl ester are added dropwise to a suspension of 317 mg of NaH (55% in mineral oil) in 30 ml of THF at 0°. The mixture obtained is stirred, 1.37 g of 3,3-dimethyl-butyric acid 4-oxo-adamantan-1-yl ester in 10 ml of THF are added slowly and the mixture obtained is stirred at RT overnight. The mixture obtained is diluted with EtAc and the diluted mixture obtained is washed with 1M aqueous NaH₂PO₄ and saturated aqueous NaHCO₃ solution. The organic layer obtained is dried, solvent is evaporated and the evaporation residue obtained is subjected to chromatography on silica gel. 3,3-Dimethyl-butyric acid 4-(fluoro-ethoxycarbonyl-methylene)-adamantan-1-yl ester is obtained.

¹³C-NMR: 171.54, 161.64, 140.78, 140.66, 139.92, 137.45, 78.28, 61.06, 49.23, 41.82, 41.80, 41.46, 40.27, 37.78, 37.54, 32.41, 32.39, 32.19, 30.72, 30.20, 29.63, 14.21.

### c. 3,3-Dimethyl-butyric acid 4-(carboxy-fluoro-methylene)-adamantan-1-yl ester

3,3-dimethyl-butyric acid 4-(fluoro-ethoxycarbonyl-methylene)-adamantan-1-yl ester is saponified analogously to the method as described in example J c. 3,3-Dimethyl-butyric acid 4-(carboxy-fluoro-methylene)-adamantan-1-yl ester is obtained.

¹³C-NMR: 172.09, 166.50, 166.13, 144.79, 144.67, 139.55, 137.13, 78.52, 49.62, 42.22, 42.20, 41.83, 40.55, 38.31, 37.96, 33.12, 33.10, 32.95, 32.87, 31.94, 31.15, 30.52, 30.10, 30.04.

### d. 3,3-Dimethyl-butyric acid 4-[2-(4-bromo-2,5-dichloro-thiophene-3-sulfonylamino)-1-fluoro-2-oxo-ethylidene]-adamantan-1-yl ester

Coupling of 3,3-dimethyl-butyric acid 4-(carboxy-fluoro-methylene)-adamantan-1-yl ester with 4-bromo-2,5-dichloro-thiophene-3-sulfonamide and isolation is performed analogously to the method as described in Example K c. 3,3-Dimethyl-butyric acid 4-[2-(4-bromo-2,5-dichloro-thiophene-3-sulfonylamino)-1-fluoro-2-oxo-ethylidene]-adamantan-1-yl ester is obtained.

### EXAMPLE M

### [4-cis/trans-(3,5-Bis-(trifluoromethyl)-benzenesulfonaminocarbonylmethyl)-cyclohexyl]-carbamic acid tert.-butyl ester (compound of Example 331)

### a. 3,5-Bis-(trifluoromethyl)benzene-sulfonamide

An aqueous solution of NH₃ (32%) is added at RT to a solution of 3,5-bis-(trifluoromethyl)-benzene-sulfonylchloride in EtAc. The mixture obtained is stirred and two phases obtained are separated, the organic layer obtained is washed with 1 N HCl and H₂O, and dried.

Solvent of the organic solution obtained is evaporated.

3,5-Bis-trifluoromethyl-benzene sulfonamide is obtained.

### b. 4-cis/trans-(3,5-Bis-(trifluoromethyl)-benzenesulfonylaminocarbonylmethyl)-cyclohexyl]-carbamic acid tert.-butyl ester

60 mg of DMAP, 130 mg of DIEA and 192 mg of EDC are added to a solution of 293 mg of 3,5-bis-trifluoromethyl-benzene-sulfonamide and 257 mg of cis/trans-1-(tert.butyloxy-carbonylamino)cyclohexane-4-acetic acid in 10 ml of DMF, and the mixture obtained is stirred for 16 hours at ca. 30°. Solvent from the mixture obtained is evaporated and the evaporation residue obtained is dissolved in EtAc. The solution obtained is washed with 1 N HCl, saturated NaHCO₃ solution and brine, and dried. From the organic phase obtained solvent is evaporated and the evaporation residue obtained is subjected to chromatography. [4-cis/trans-(3,5-bis-(trifluoromethyl)-benzenesulfonylaminocarbonylethyl)-cyclohexyl]-carbamic acid tert.-butyl ester in the form of an isomeric mixture is obtained.

### EXAMPLE N

### 1-[2-(3,5-Bis-trifluoromethyl-benzenesulfonylamino)-2-oxo-(4-chloro-phenyl)-ethyl]-piperidine-4-carboxylic acid cyclohexylamide (compound of Example 371)

140 mg of triethylamine and 0.32 ml of 50% propylphosphonic acid anhydride (solution in DMF) are added to a solution of 150 mg of (4-chlorophenyl)-(4-cyclohexylcarbamoyl-piperidin1-yl)-acetic acid, 174 mg of 3,5-bis(trifluoromethyl)-benzenesulfonamide and 24 mg of DMAP in 6 ml of anhydrous DMF at 10°. The mixture obtained is stirred for ca. 60 hours at RT, solvent is evaporated off and the evaporation residue obtained is treated with EtAc and H₂O. Two phases obtained are separated and the organic layer obtained is washed, dried and solvent is evaporated. The evaporation residue obtained is subjected to chromatography on silica gel.

1-[2-(3,5-Bis-trifluoromethyl-benzenesulfonylamino)-2-oxo-(4-chloro-phenyl)-ethyl]-piperidine-4-carboxylic acid cyclohexylamide is obtained.

### EXAMPLE O

### 1-[2-Benzenesulfonylamino-1-(3,5-bistrifluoromethyl-phenyl)-2-oxo-ethyl]-piperidine-4-carboxylic acid cyclohexylamide (compound of Example 365)

A solution of 500 mg of bromo-(4-chlorophenyl)-acetic acid methyl ester in 1.3 ml of CH₃CN is added to a solution of 288 mg piperidine-4-carboxylic acid cyclohexylamide and 0.239 ml DIEA in 4 ml of CH₃CN at RT, the mixture obtained is stirred for ca. 24 hours at RT, solvent is evaporated and the evaporation residue obtained is treated with EtAc and H₂O. The organic phase obtained is washed, dried and solvent is evaporated.

1-[2-Benzenesulfonylamino-1-(3,5-bistrifluoromethyl-phenyl)-2-oxo-ethyl]-piperidine-4-carboxylic acid cyclohexylamide is obtained.

### EXAMPLE P (COMPOUND OF EXAMPLE 375)

### 4-(1-Carboxy-cyclopentyl)-piperidine-1-carboxylic acid tert-butyl ester

### a. 1-Pyridin-4-yl-cyclopentanecarboxylic acid ethyl ester

25 ml of a n-butyllithium solution in HEX (1.6M) is slowly added to a solution of 2.17 ml of pyridin-4-yl-acetic acid ethyl ester in 200 ml of THF, the mixture obtained is stirred at RT for 30 minutes, is cooled to −78° and treated with 2.8 ml of 1,4-dibromobutane in 20 ml of THF. The mixture obtained is allowed to warm up to RT overnight, is treated with EtAc, the organic layer obtained is washed with H₂O, saturated NaHCO₃ solution and brine, dried and solvent is evaporated. The evaporation residue obtained is subjected to chromatography.

1-Pyridin-4-yl-cyclopentanecarboxylic acid ethyl ester is obtained.

¹³C-NMR: 175.05, 152.68, 150.15, 122.44, 61.63, 59.18, 36.19, 24.06, 14.33.

### b. 1-Piperidin-4-yl-cyclopentanecarboxylic acid ethyl ester in the form of a hydrochloride

1.75 g of 1-pyridin-4-yl-cyclopentanecarboxylic acid ethyl ester are dissolved in a mixture of 100 ml of MeOH and aqueous HCl (32%) and the mixture obtained is hydrogenated in the presence of 175 mg of PtO₂ as a catalyst under pressure for 5 hours. From the mixture obtained the catalyst is removed and solvent is evaporated. 1-Piperidin-4-yl-cyclopentanecarboxylic acid ethyl ester in the form of a hydrochloride salt is obtained. ¹³C-NMR (CD₃OD): 176.73, 61.33, 57.71, 45.08, 45.00, 42.14, 33.80, 25.49, 25.43, 25.36, 14.58.

### c. 4-(1-Ethoxycarbonyl-cyclopentyl)-piperidine-1-carboxylic acid tert-butyl ester

2.0 g of 1-piperidin-4-yl-cyclopentanecarboxylic acid ethyl ester in the form of a hydrochloride are converted into 4-(1-ethoxycarbonyl-cyclopentyl)-piperidine-1-carboxylic acid tert-butyl ester analogously to the procedure as described in Example F, c.

4-(1-Ethoxycarbonyl-cyclopentyl)-piperidine-1-carboxylic acid tert-butyl ester is obtained. ¹³C-NMR: 177.22, 155.16, 79.67, 60.75, 58.22, 44.77, 44.46, 33.73, 28.83, 28.67, 25.34, 14.66.

### d. 4-(1-Carboxy-cyclopentyl)-piperidine-1-carboxylic acid tert-butyl ester

A solution of 1.2 g of 4-(1-ethoxycarbonyl-cyclopentyl)-piperidine-1-carboxylic acid tert-butyl ester in a mixture of 100 ml of EtOH and 50 ml of an 1M aqueous NaOH is stirred at 70° for 14 days, EtAc is added and two phases obtained are separated. The aqueous layer obtained is acidified with HCl (pH 2-3) and extracted with EtAc. The organic layer obtained is washed with brine, dried and solvent is evaporated.

4-(1-Carboxy-cyclopentyl)-piperidine-1-carboxylic acid tert-butyl ester is obtained.

### EXAMPLE Q

### 4-[(3,5-bis-trifluoromethyl-benzoylsulfamoyl)-methyl]-piperidine-1-carboxylic acid tert-butyl ester (compound of Example 378)

### a. 4-[(benzhydryl-sulfamoyl)-methyl]-4-hydroxy-piperidine-1-carboxylic acid tert.-butyl ester

28 ml of n-butyllithium (1.6 N solution in HEX) are added at −70° to a solution of 5.22 g of N-(diphenylmethyl)-methanesulfonamide in 120 ml of THF. The mixture is warmed to 0°, cooled to −30° and treated with 4 g of BOC-piperidin-4-one in 15 ml of THF. The mixture obtained is stirred at RT overnight, solvent is evaporated, the evaporation residue obtained is treated with EtAc, washed with 1 N HCl, saturated, aqueous NaHCO₃ solution and brine, the organic layer obtained is dried and solvent is evaporated. The evaporation residue obtained is subjected to chromatography on silica gel. 4-[(Benzhydryl-sulfamoyl)-methyl]-4-hydroxy-piperidine-1-carboxylic acid tert.-butyl ester is obtained. m.p. 121-123°.

### b. 4-Hydroxy-4-sulfamoylmethyl-piperidine-1-carboxylic acid tert.-butyl ester

5.19 g of 4-[(benzhydryl-sulfamoyl)-methyl]4-hydroxy-piperidine-1-carboxylic acid tert.-butyl ester in 150 ml of MeOH are treated with 100 μl of triethylamine and the mixture obtained is hydrogenated overnight at RT with 10% Pd/C as a catalyst. From the mixture obtained the catalyst is filtrated off, solvent is evaporated and the evaporation residue is subjected to chromatography on silica gel. 4-Hydroxy-4-sulfamoylmethyl-piperidine-1-carboxylic acid tert.-butyl ester are obtained. m.p. 176-180°.

### c. 4-[(3,5-bis-trifluoromethyl-benzoylsulfamoyl)-methyl]-4-hydroxy-piperidine-1-carboxylic acid tert-butyl ester

1510 mg of 3,5-bis-(trifluoromethyl)-benzoic acid, 477 mg of DMAP, 1010 mg of DIEA and 1500 mg of EDC are added to a solution of 1150 mg of 4-hydroxy-4-sulfamoylmethyl-piperidine-1-carboxylic acid tert-butyl ester. The mixture obtained is stirred for 16 hours, solvent is evaporated and the evaporation residue is treated with EtAc, washed with 1 N HCl, saturated, aqueous NaHCO₃ solution and brine, the organic layer obtained is dried and subjected to chromatography on silica gel. 4-[(3,5-bis-trifluoromethyl-benzoylsulfamoyl)-methyl]-4-hydroxy-piperidine-1-carboxylic acid tert-butyl ester is obtained. m.p. 154-159°.

### d. 4-[(3,5-bis-trifluoromethyl-benzoylsulfamoyl)-methylene]-piperidine-1-carboxylic acid tert.-butyl ester

1510 mg of Martin Sulfurane dehydrating agent are added to 300 mg of 4-[(3,5-bis-trifluoromethyl-benzoylsulfamoyl)-methyl]-4-hydroxy-piperidine-1-carboxylic acid tert.-butyl ester in 5 ml of CH₂Cl₂. The mixture obtained is stirred in a microwave oven at 100° for 15 minutes, from the mixture obtained solvent is evaporated and the evaporation residue is subjected to chromatography on silica gel.

4-[(3,5-bis-trifluoromethyl-benzoylsulfamoyl)-methylene]-piperidine-1-carboxylic acid tert.-butyl ester is obtained. m.p. 132-136°.

### e. 4-[(3,5-bis-trifluoromethyl-benzoylsulfamoyl)-methyl]-piperidine-1-carboxylic acid tert-butyl ester

A solution of 880 mg of 4-[(3,5-bis-trifluoromethyl-benzoylsulfamoyl)-methylene]-piperidine-1-carboxylic acid tert.-butyl ester in 100 ml of MeOH is hydrogenated (10% Pd/C as a catalyst). From the mixture obtained the catalyst is filtrated off and solvent is evaporated. 4-[(3,5-Bis-trifluoromethyl-benzoylsulfamoyl)-methyl]-piperidine-1-carboxylic acid tert-butyl ester is obtained.

Analogously to methods as described in the PROCEDURES (Examples A to Q), but using appropriate starting materials, compounds of formula

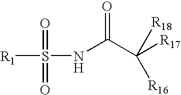

wherein R₁₈ is hydrogen and R₁ and R₁₆+R₁₇ are as defined in TABLE 1 (compounds of formula I, wherein m is 0, n is 0, and R₁ is a group of formula VII) are obtained, if not otherwise indicated in TABLE 1. If not otherwise indicated, in TABLE 1 ¹³C-NMR and ¹H-NMR data are determined in CDCl₃.

TABLE 1

| EX | R₁ | R₁₆ + R₁₇ | m.p./¹H-NMR/¹³C-NMR |
|---|---|---|---|
| 1 | 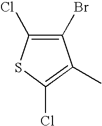 | 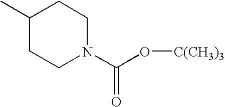 | (DMSO-d₆): δ = 1.40 (s, 9H); 1.41-1.82(m, 4H); 2.42 (m, 1H), 2.78 (t, 2H);4.08 (d, 2H) |
| 2 | 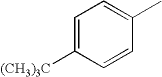 | 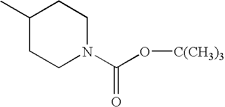 | 1.20-1.38 (m, 2H); 1.30 (s, 9H); 1.64(d, 2H); 2.35 (m, 1H); 2.60-2.80 (m,2H); 3.82 (d, 2H); 7.58 +7.78 (2m, 4H) |
| 3 | 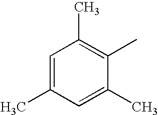 | 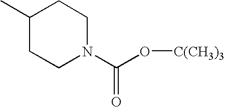 | 1.41 (s, 9H); 1.43-1.80 (m, 2H); 2.35(s, 3H); 2.34-2.42 (m, 1H); 2.72 (s,6H); 2.60-2.80 (m, 2H); 3.98-4.14 (m,2H); 6.98 (s, 2H); 8.98 (s, 1H) |
| 4 | 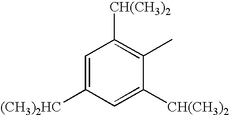 | 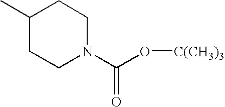 | 1.24; 1.26; 1.28; 1.29; 1.32 (5s, 18H);1.43 (s, 9H); 1.45-1.78 (m, 5H); 1.70 (t,2H); 2.91 (sep, 1H); 4.03-4.25(m + sep, 4H); 7.24 (s, 2H); 8.44 (s,1H) |
| 5 | 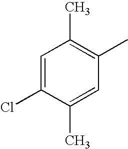 | 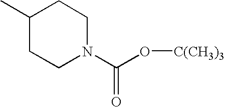 | 1.40 (s, 9H); 1.40-1.60 (m, 2H); 1.72(m, 2H); 2.38 (m, 1H); 2.40 (s, 3H);2.56 (s, 3H); 2.72 (t, 2H); 4.04 (d, 2H);7.22 (s, 1H); 7.98 (s, 1H) |
| 6 | 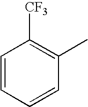 | 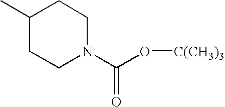 | 1.41 (s, 9H); 1.41-1.82 (m, 4H); 2.38(m, 1H), 2.75 (t, 2H); 4.08 (d, 2H);7.58-7.81 (m, 2H); 7.85 (m, 1H); 8.50(m, 1H) |
| 7 | 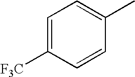 | 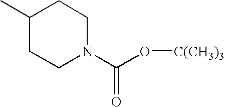 | 1.42 (s, 9H); 1.45-1.90 (m, 4H); 2.35(m, 1H); 2.78 (t, 2H); 4.05 (d, 2H);8.30 (broad, 4H) |
| 8 | 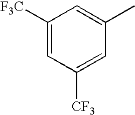 | 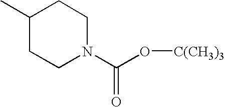 | 1.41 (s, 9H); 1.45-1.68 (m, 2H); 1.80(m, 2H); 2.30-2.40 (m, 1H); 2.80 (t,2H); 4.10 (d, 2H); 8.15 (s, 1H); 8.40 (s,1H); 8.54 (s, 2H). 1.40 (s, 9H);1.40-1.60 (m, 2H); 1.72 (m, 2H); 2.30(m, 2H); 3.88 (s, 3H); 4.04 (d, 2H) |
| 9 | 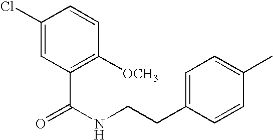 | 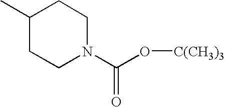 | 1.12-1.36 (m, 2H); 1.40 (s, 9H); 1.63(d, 2H); 2.36-2.42 (m, 1H); 2.60-2.80(m, 2H); 2.96 (t, 2H); 3.55 (q, 2H);3.80 (s, 3H); 3.84 (d, 2H); 7.18 (d, 1H);7.46-7.52 (m, 3H); 7.61 (d, 1H); 7.81(d, 1H); 8.24 (d, 1H) |
| 10 | 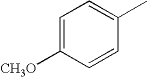 | 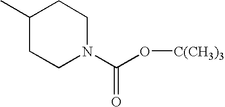 | 1.40 (s, 9H); 1.40-1.60 (m, 2H); 1.72(m, 2H); 2.30 (m, 2H); 3.88 (s, 3H);4.04 (d, 2H); 6.95 (d, 2H); 7.90 (2, 2H) |
| 11 | 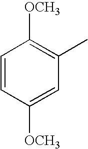 | 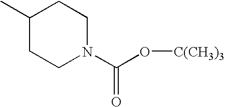 | 1.40 (s, 9H); 1.40-1.60 (m, 2H); 1.72(m, 2H); 2.38 (m, 1H); 2.72 (t, 2H);3.85 (s, 3H); 4.00 (s, 3H); 4.04 (d, 2H);6.98 (d, 1H); 7.18 (dd, 1H); 7.60 (d,1H) |
| 12 | 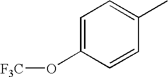 | 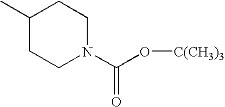 | 1.41 (s, 9H); 1.56-1.90 (m, 4H); 2.30(m, 1H); 2.72 (t, 2H); 4.04 (d, 2H);7.34 (d, 2H); 8.10 (d, 2H); 8.22 (s, 1H) |
| 13 | 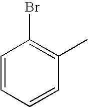 | 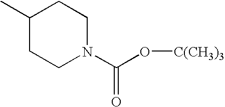 | 1.41 (s, 9H); 1.50-1.90 (m, 4H); 2.40(m, 1H); 2.78 (t, 2H); 4.04 (d, 2H);7.41-7.59 (m, 2H); 7.74 (d, 1H); 8.28(d, 1H); 8.60 (s, 1H) |
| 14 | 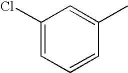 | 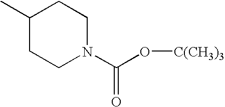 | 1.18-1.38 (m, 2H); 1.40 (s, 9H); 1.70(d, 2H); 2.38-2.45 (m, 1H); 2.60-2.80(m, 2H); 3.82 (d, 2H); 7.62 + 7.90 (2m,4H) |
| 15 | 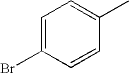 | 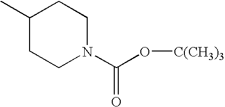 | 1.20-1.38 (m, 2H); 1.40 (s, 9H); 1.65(d, 2H); 2.40 (m, 1H); 2.60-2.80 (m,2H); 3.84 (d, 2H); 7.80 +7.83 (2m, 4H) |
| 16 | 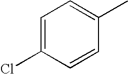 | 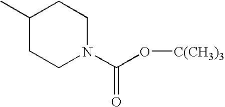 | 1.20-1.35 (m, 2H); 1.40 (s, 9H); 1.63(d, 2H); 2.41 (m, 1H); 2.73 (t, 2H);3.90 (d, 2H); 7.70 + 7.90 (2 m, 4H) |
| 17 | 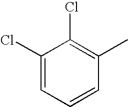 | 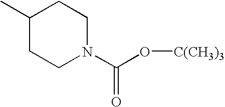 | 1.40 (s, 9H); 1.40-1.60 (m, 2H); 1.72(m, 2H); 2.38 (m, 1H); 2.72 (t, 2H);4.04 (d, 2H); 7.38 (t, 1H); 7.62 (d, 1H);8.13 (d, 1H) |
| 18 | 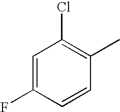 | 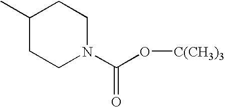 | 1.41 (s, 9H); 1.38-1.90 (m, 4H); 2.39(m, 1H); 2.78 (t, 2H); 4.06 (d, 2H);7.13-7.30 (m, 2H); 8.26 (m, 1H) |
| 19 | 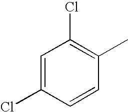 | 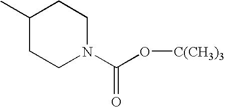 | 1.41 (s, 9H); 1.40-1.93 (m, 4H); 2.40(m, 1H); 2.80 (t, 2H); 4.08 (d, 2H);7.50 (dd, 1H); 7.54 (d, 1H); 8.18 (d,1H); 8.58 (s, 1H) |
| 20 | 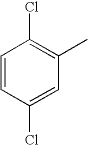 | 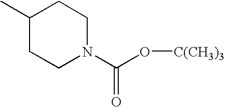 | 1.40 (s, 9H); 1.40-1.60 (m, 2H); 1.72(m, 2H); 2.38 (m, 1H); 2.72 (t, 2H);4.04 (d, 2H); 7.38-7.50 (m, 2H); 8.18(m, 1H) |
| 21 | 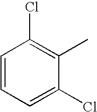 | 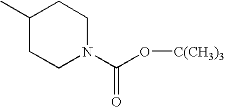 | 1.41 (s, 9H); 1.41-1.85 (m, 4H); 2.40(m, 1H); 2.78 (t, 2H); 4.08 (d, 2H);7.36-7.54 (m, 3H) |
| 22 | 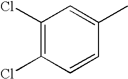 | 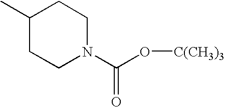 | 1.43 (s, 9H); 1.44-1.95 (m, 4H); 2.31(m, 1H); 3.76 (t, 2H); 4.08 (d, 2H);7.62 (d, 1H); 7.90 (d, 1H); 8.18 (d, 1H) |
| 23 | 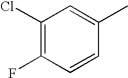 | 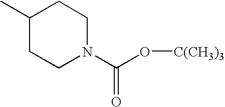 | 1.41 (s, 9H); 1.41-1.88 (m, 4H); 2.30(m, 1H); 2.74 (t, 2H); 4.06 (d, 2H);7.22 (m, 1H); 7.98 (m, 1H); 8.04 (m,1H); 8.30 (s, 1H) |
| 24 | 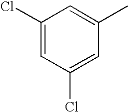 | 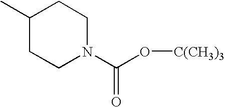 | 1.42 (s, 9H); 1.35-1.90 (m, 4H); 2.38(m, 1H); 2.76 (t, 2H); 4.02 (m, 2H);7.56 (s, 1H); 7.81 (s, 2H) |
| 25 | 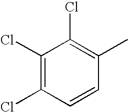 | 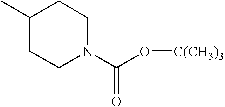 | 1.41 (s, 9H); 1.40-1.91 (m, 4H); 2.38(m, 1H); 2.78 (t, 2H); 4.08 (d, 2H);7.01 (d, 1H); 8.14 (d, 1H); 8.42 (s, 1H) |
| 26 | 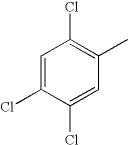 | 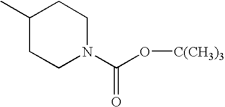 | 1.41 (s, 9H); 1.38-1.88 (m, 4H); 2.40(m, 1H); 2.78 (t, 2H); 4.10 (d, 2H);7.61 (s, 1H); 8.32 (s, 1H); 8.42 (s, 1H) |
| 27 | 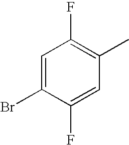 | 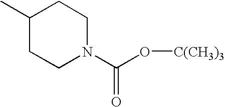 | 0.90 (m, 1H); 1.20-1.90 (m, 3H); 1.43(s, 9H); 2.40 (m, 1H); 2.80 (t, 2H); 4.10(d, 2H); 7.43 (dd, 1H); 7.83 (dd, 1H);8.48 (s, 1H) |
| 28 | 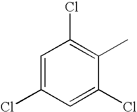 | 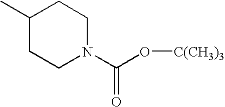 | 1.40 (s, 9H); 1.40-1.90 (m, 4H); 2.40(m, 1H); 2.78 (t, 2H); 4.08 (d, 2H);7.50 (s, 2H); 8.84 (s, 1H) |
| 29 | 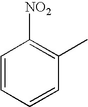 | 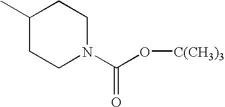 | 1.40 (s, 9H); 1.40-1.60 (m, 4H); 1.72(m, 2H); 2.40 (m, 1H); 2.80 (t, 2H);4.04 (d, 2H); 7.78-7.82 (m, 3H); 8.42(m, 1H) |
| 30 | 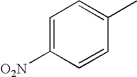 | 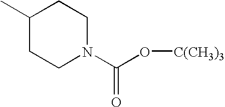 | 1.42 (s, 9H); 1.42-1.86 (m, 4H); 2.35(m, 1H); 2.74 (t, 2H); 4.04 (d, 2H);8.22 and 8.38 (AB, 4H); 8.42 (s, 1H) |
| 31 | 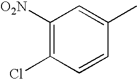 | 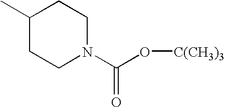 | 1.42 (s, 9H); 1.40-1.96 (m, 6H); 1.38(m, 1H); 1.79 (t, 2H); 4.10 (d, 2H);7.75 (d, 1H); 8.23 (dd, 1H); 8.50 (d,1H); 8.62 (s, 1H) |
| 32 | 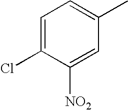 | 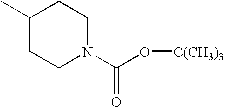 | 1.40 (s, 9H); 1.42-1.90 (m, 4H); 2.38(m, 1H); 2.78 (t, 2H); 4.10 (d, 2H);7.72 (d, 1H); 8.21 (dd, 1H); 8.41 (s,1H); 8.50 (d, 1H) |
| 33 | 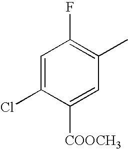 | 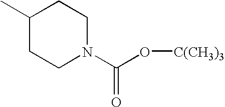 | 8.22 (d, J = 7.6 Hz, 1H), 7.61 (d,J =13.9 Hz, 1H), 3.87 (s, 3H),3.73-3.82 (m, 2H), 2.65-2.77 (br.s, 1H),2.07-2.16 (br.s, 1H), 1.56-1.63 (m, 2H),1.36 (s, 9H), 1.17-1.29 (m, 2H) |
| 34 | 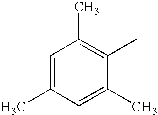 | 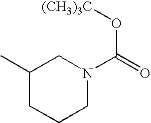 | 1.44 (s, 9H); 1.65-1.99 (m, 4H); 2.30(s, 3H); 2.40 (m, 1H); 2.70 (s, 6H);3.02-3.30 (2m, 2H); 3.54-3.82 (2m,2H); 7.24 (s, 2H) |
| 35 | 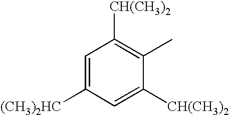 | 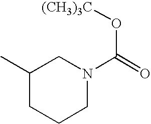 | 1.18-1.35 (m, 18H); 1.48 (s, 9H);1.44-1.94 (m, 4H); 2.40 (m, 1H); 2.90(sep, 1H); 3.08-3.19 (2m, 2H);3.51-3.63 (2m, 2H); 4.20 (sep, 2H);7.07 (s, 1H); 7.18 (s, 2H) |
| 36 | 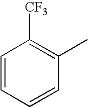 | 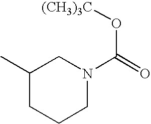 | 1.43 and 1.48 (2s, 9H); 7.78 (m, 2H);7.80 (m, 1H); 8.50 (m, 1H) (mixture ofrotamers) |
| 37 | 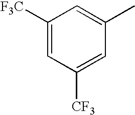 | 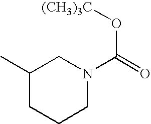 | 1.35-1.60 (m, 11H); 1.70-2.20 (m, 2H);2.50 (m, 1H); 3.20-3.40 (m, 4H); 8.10(s, 1H); 8.55 (s, 2H) |
| 38 | 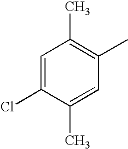 | 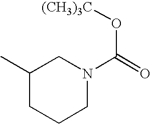 | 1.40-1.55 (m, 11H); 1.80 (m, 2H); 2.40(s, 3H); 2.42 (m, 1H); 2.60 (s, 3H);3.10-3.80 (m, 4H); 7.22 (s, 1H); 8.00(s, 1H) |
| 39 | 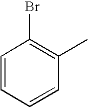 | 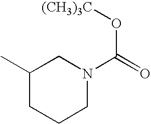 | 1.42 and 1.50 (2s, 9H), 7.40-7.50 (m,2H); 7.63 (dd, 1H); 8.28 (dd, 1H)(mixture of rotamers) |
| 40 | 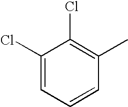 | 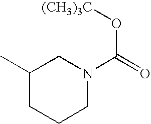 | 1.50 (m, 11H); 2.50 (m, 1H); 3.20-3.60(m, 3H); 3.70 (m, 1H); 7.40 (t, 1H);7.50 (d, 1H); 8.20 (d, 1H) |
| 41 | 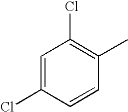 | 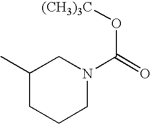 | 1.50 (s, 9H); 1.78-2.00 (m, 4H); 2.46(m, 1H); 3.18-3.58 (m, 3H); 3.62-3.78(m, 1H); 7.43 (dd, 1H); 7.54 (d, 1H);8.19 (d, 1H) |
| 42 | 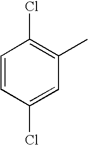 | 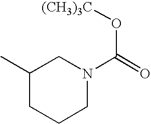 | 1.43 (s, 9H); 1.50 (m, 2H); 1.90 (m,2H); 2.50 (m, 1H); 3.20-3.80 (m, 4H);7.40-7.58 (m, 2H); 8.22 (d, 1H) |
| 43 | 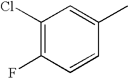 | 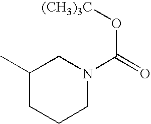 | 1.48 (s, 9H); 1.70-2.10 (m, 4H); 2.42(m, 1H); 3.40 (m, 2H); 3.58 (m, 2H);7.20-7.29 (m, 1H); 7.98 (ddd, 1H); 8.10(dd, 1H) |
| 44 | 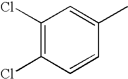 | 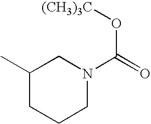 | 1.52 (s, 9H); 1.60-2.15 (m, 4H); 2.51(m, 1H); 3.30-3.72 (m, 4H); 7.60 (d,1H); 7.86 (dd, 1H); 8.10 (d, 1H) |
| 45 | 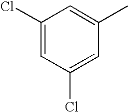 | 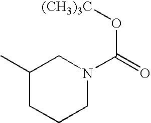 | 1.51 (s, 9H); 1.62-2.16 (m, 4H); 2.50(m, 1H); 3.35-3.66 (m, 4H); 7.58 (t,1H); 7.94 (d, 2H) |
| 46 | 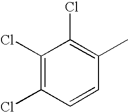 | 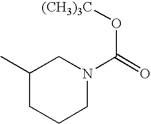 | 1.50 (s, 9H); 1.79-1.99 (m, 4H); 2.51(m, 1H); 3.27-3.72 (m, 4H); 7.58 (d,1H); 8.10 (d, 1H) |
| 47 | 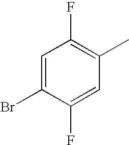 | 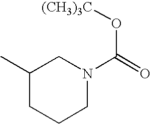 | 1.50 (s, 9H); 1.75-2.02 (m, 4H); 2.53(m, 1H); 3.22-3.80 (m, 4H); 7.48 (dd,1H); 7.82 (dd, 1H) |
| 48 | 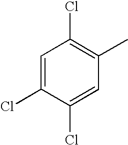 | 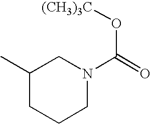 | 1.50 (s, 9H); 1.70-2.02 (m, 4H); 2.50(m, 1H); 3.22-3.38 (m, 1H); 3.40-3.58(m, 2H); 3.68 (m, 1H); 7.60 (s, 1H);8.34 (s, 1H) |
| 49 | 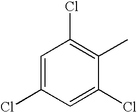 | 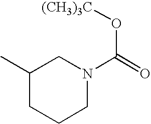 | 1.43 (s, 9H); 1.40-1.98 (m, 4H); 2.50(m, 1H); 3.23-3.40 (2m, 2H); 3.54 and3.74 (2m, 2H); 7.52 (s, 2H) |
| 50 | 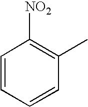 | 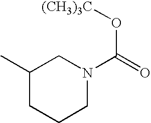 | 1.40-2.00 (m, 13H), 2.50 (m, 1H);2.98-3.20 (m, 2H); 3.70 (m, 2H); 3.98(d, 2H); 7.80 (m, 3H); 8.40 (m, 1H) |
| 51 | 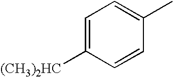 | 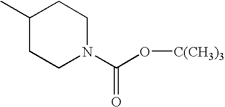 | 1.24 (d, 6H); 1.42 (s, 9H); 1.44-1.90(m, 4H); 2.35 (m, 1H); 2.78 (t, 2H);3.00 (sept, 1H); 4.05 (d, 1H); 7.38 (d,2H); 7.90 (d, 2H); 8.28 (s, 1H) |
| 52 | 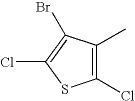 | 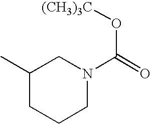 | 1.50 (s, 9H); 1.80-2.04 (m, 4H); 2.52(m, 1H); 3.21-3.78 (m, 4H) |
| 53 | 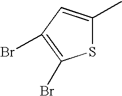 | 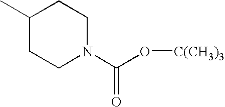 | 1.45 (s, 9H), 1.60 (dq, 2H), 1.78 (broadd, 2H), 2.32 (tt, 1H), 4.06 (broad d,2H), 7.63 (s, 1H) |
| 54 | 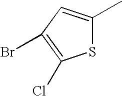 | 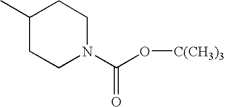 | 1.45 (s, 9H), 1.69 (dq, 2H), 1.76 (dq,2H), 2.34 (tt, 1H), 2.77 (broad t, 2H),4.05 (broad d, 2H), 7.60 (s, 1H) |
| 55 | 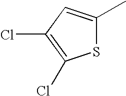 | 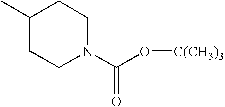 | 1.45 (s, 9H), 1.59 (dq, 2H), 1.77 (dq,2H), 2.38-2.43 (m, 3H), 2.76 (broad t,2H), 4.06 (d, 2H), 7.63 (s, 1H) |
| 56 | 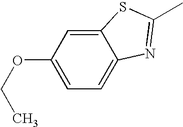 | 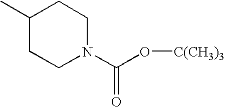 | 1.20-1.38 (m, 2H); 1.40-1.42 (m, 12H);1.75 (d, 2H); 2.40-2.55 (m, 1H);2.62-2.82 (m, 2H); 3.84 (d, 2H); 4.18(q, 2H); 7.23 (dd, 1H); 7.81 (d, 1H);8.08 (d, 1H) |
| 57 | 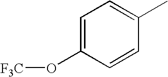 | 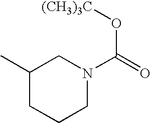 | 1.43 (s, 9H); 1.43-2.10 (m, 4H); 2.42(m, 1H); 3.26-3.59 (m, 4H); 7.30 (d,2H); 8.08 (d, 2H) |
| 58 | 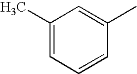 | 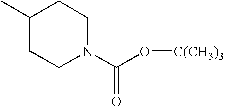 | 1.44 (s, 9H); 1.52-1.61 (m, 2H); 1.76(m, 2H); 2.31 (m, 1H); 2.46 (s, 3H);2.73 (m, 2H); 4.05 (broad, 2H);7.41-7.49 (m, 2H); 7.82-7.88 (m, 2H);8.30 (bs, 1H) |
| 59 | 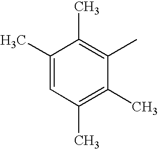 | 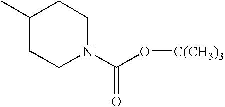 | (DMSO-d₆): 1.32 (m, 2H); 1.43 (s, 9H);1.76 (m, 2H); 2.32 (s, 6H); 2.52 (m,1H); 2.70-2.82 (broad, 2H); 3.40 (s,6H); 3.95 (d, 2H); 7.35 (s, 1H) |
| 60 | 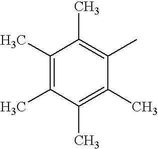 | 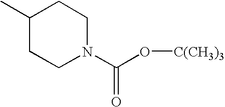 | (DMSO-d₆): 1.22 (m, 2H); 1.38 (s, 9H);1.66 d, 2H); 2.18 (s, 6H); 2.22 (s, 3H);2.42 (m, 1H); 2.54 (s, 6H); 2.59-2.76(m, 2H); 3.87 (d, 2H); 12.08 (bs, 1H) |
| 61 | 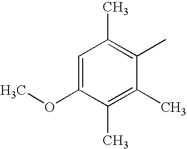 | 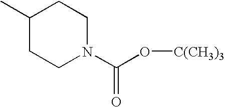 | (DMSO-d₆): 1.02 (m, 2H); 1.16 (s, 9H);1.44 (m, 2H); 1.87 (s, 3H); 2.12-2.25(m, 1H); 2.43 (s, 3H); 2.48 (broad, 2H);3.61 (s, 3H); 3.65 (d, 2H); 6.60 (s, 1H);11.83 (bs, 1H) |
| 62 | 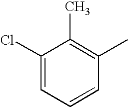 | 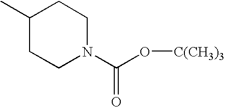 | 1.44 (s, 9H); 1.53 (m, 2H); 1.74 (m,2H); 2.35 (m, 1H); 2.66 (s, 3H); 2.75(m, 2H); 4.03 (d, 2H); 7.32 (dt, 1H);7.62 (dd, 1H); 8.11 (dd, 1H) |
| 63 | 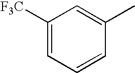 | 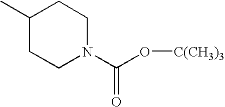 | 1.43 (s, 9H); 1.53 (m, 2H); 1.72 (m,2H); 2.31 (m, 1H); 2.73 (m, 2H); 4.01(m, 2H); 7.70 (t, 1H); 7.99 (d, 1H);8.26-8.30 (m, 2H) |
| 64 | 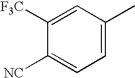 | 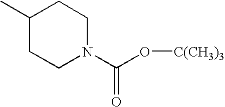 | DMSO-d₆: 1.10 (m, 2H); 1.23 (s, 9H);1.48 (m, 2H); 1.97 (m, 1H); 2.50-2.64(broad, 2H); 3.60 (d, 2H); 8.02 (dd,1H); 8.05 (d, 1H); 8.10 (d, 1H) |
| 65 | 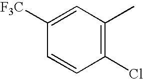 | 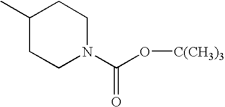 | CDCl₃ + 5% CD₃OD: 1.44 (s, 9H); 1.53(m, 2H); 1.78 (d, 2H); 2.41 (m, 1H);2.78 (m, 2H), 4.03 (m, 2H); 7.67 (d,1H); 7.81 (dd, 1H); 8.51 (d, 1H) |
| 66 | 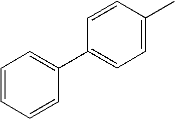 | 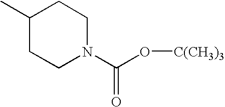 | (DMSO-d₆): 1.03 (m, 2H); 1.45 (m,2H); 2.18 (m, 1H); 2.41-2.52 (m, 2H);3.63 (d, 2H); 7.30-7.35 (m, 1H); 7.40(t, 2H); 7.53 (d, 2H); 7.67 and 7.72(AB, 4H) |
| 67 | 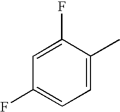 | 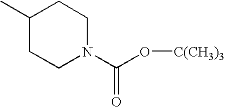 | 1.44 (s, 9H); 1.57 (m, 2H); 1.79 (m,2H); 2.37 (m, 1H); 2.77 (m, 2H); 4.07(broad, 2H); 6.97 (m, 1H); 7.08 (m,1H); 8.12 (m, 1H), 8.45-8.85 (broad,1H) |
| 68 | 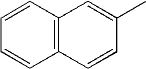 | 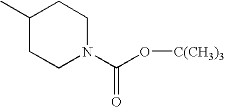 | CDCl₃ + 5% CD₃OD: 1.42 (s, 9H); 1.50(m, 2H); 1.71 (m, 2H); 2.34 (m, 1H);2.75 (m, 2H); 7.60-7.70 (m, 2H);7.90-8.05 (m, 4H); 8.63 (s, 1H) |
| 69 | 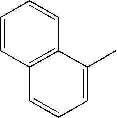 | 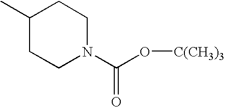 | 1.34-1.44 (m, 9 + 2H); 1.61 (m, 2H);2.29 (m, 1H); 2.67 (t, 2H); 3.91 (dt,2H); 7.57-7.63 (m, 2H); 7.67 (m, 1H);7.96 (dd, 1H); 8.12 (d, 1H); 8.48 (dd,1H); 8.58 (dd, 1H) |
| 70 | 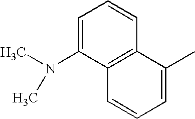 | 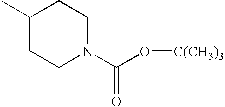 | CDCl₃ + 5% CD₃OD: 1.39 (s, 9H); 1.42(m, 2H); 1.62 (m, 2H); 2.29 (m, 1H);2.67 (m, 2H); 2.90 (s, 6H); 3.93 (m,2H); 7.16 (d, 1H); 7.52-7.61 (m, 2H);8.19 (d, 1H); 8.48 (dd, 1H); 8.59 (d,1H) |
| 71 | 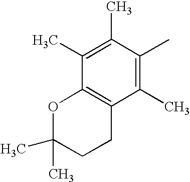 | 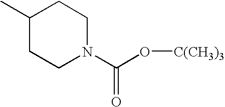 | (DMSO-d₆): 0.99 (m, 2H); 1.04 (s, 6H);1.13 (s, 9H); 1.43 (m, 2H); 1.56 (t, 2H);1.83 (s, 3H); 2.15-2.23 (m, 1H);2.24-2.27 (m, 5H); 3.39 (t, 2H);2.42-2.48 (broad, 2H); 3.65 (d, 2H) |
| 72 | 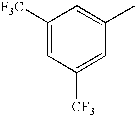 | 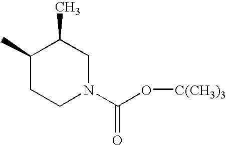 | 141.53, 133.45, 133.10, 129.33, 128.00,80.35, 32.06, 28.74 (cis) |
| 73 | 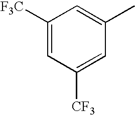 | 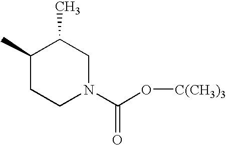 | 154.89, 141.61, 133.44, 133.10, 129.27,127.92, 124.04, 121.33, 80.71, 67.48,51.98, 33.31, 28.77, 16.90 (trans) |
| 74 | 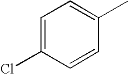 | 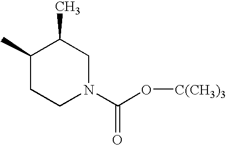 | 171.63, 155.41, 141.28, 137.19, 130.31,128.72, 80.20, 67.48, 46.34, 32.05,28.76, 13.01 (cis) |
| 75 | 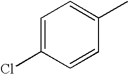 | 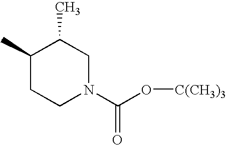 | 172.36, 154.83, 141.31, 137.18, 130.26,129.75, 80.42, 51.87, 33.38, 28.76,17.04 (trans) |
| 76 | 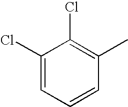 | 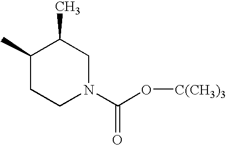 | 171.78, 155.40, 138.26, 136.08, 135.90,132.07, 130.47, 128.10, 80.16, 67.48,46.49, 31,95, 28.76, 12.93 (cis) |
| 77 | 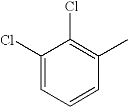 | 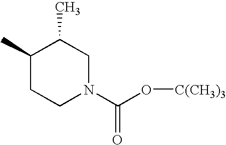 | 172.34, 154.77, 138.28, 136.11, 135.95,132.01, 128.09, 80.39, 67.58, 51.98,33.17, 28.77, 17.08 (trans) |
| 78 | 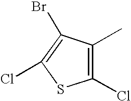 | 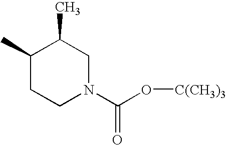 | 172.08, 155.42, 137.67, 131.09, 126.31,108.53, 80.22, 67.48, 46.58, 31.89,28.78, 13.07 (cis) |
| 79 | 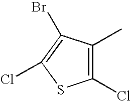 | 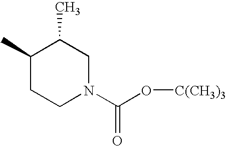 | 172.85, 154.79, 108.49, 80.43, 67.48,51.87, 33.16, 28.79, 17.21 (trans) |
| 80 | 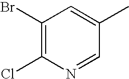 | 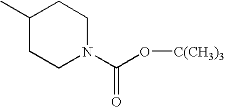 | 1.45 (s, 9H), 1.55 (dq, 2H), 1.75 (broadd, 2H), 2.32 (tt, 1H), 2.75 (bt, 2H), 4.05(broad d, 2H), 8.58 (d, 1H), 8.88 (d,1H) |
| 81 | 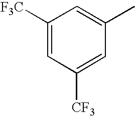 | 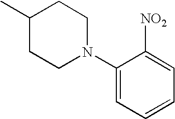 | δ = 1.80-1.95 (m, 4H); 2.32-2.40 (m,1H); 2.73-2.83 (m, 2H); 3.22 (bd, 2H);6.98 (t, 1H); 7.08 (d, 1H); 7.42 (dt,1H); 7.71 (dd, 1H); 7.94 (s, 1H); 8.48(s, 2H) |
| 82 | 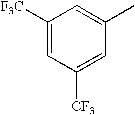 | 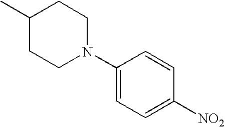 | 1.40-1.52 (m, 2H); 1.68-1.76 (m, 2H);2.56 (m, 1H); 3.03 (dt, 2H); 3.98 (dt,2H); 6.98 (d, 2H); 8.00 (d, 2H); 8.17 (s,1H); 8.25 (s, 2H) |
| 83 | 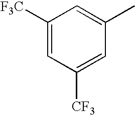 | 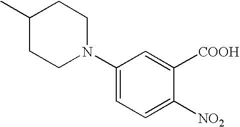 | 224-227° |
| 84 | 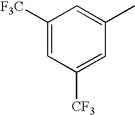 | 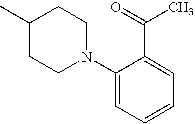 | (DMSO-d₆): 1.57 (dq, 2H), 1.79 (broadd, 2H), 2.31 (tt, 1H), 2.51 (s, 3H), 2.66(dt, 2H), 3.07 (dt, 2H), 7.02 (t, 1H),7.10 (d, 1H), 7.29 (dd, 1H), 7.40 (dt,1H), 8.39 (s, 2H), 8.49 (s, 1H) |
| 85 | 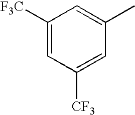 | 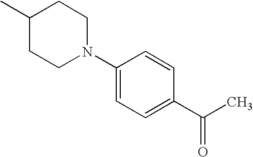 | (DMSO-d₆): 1.43 (dq, 2H), 1.70 (dd,2H), 2.20 (m, 1H), 2.40 (s, 3H), 2.84 (t,2H), 3.79 (m, 2H), 4.05 (broad, 1H,NH), 6.90 (d, 2H), 7.73 (d, 2H), 8.20(s, 1H), 8.25 (s, 2H) |
| 86 | 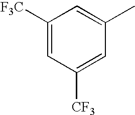 | 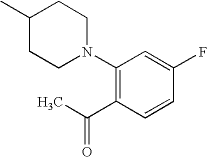 | 189-192° |
| 87 | 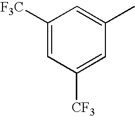 | 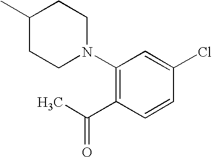 | 81-83° |
| 88 | 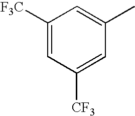 | 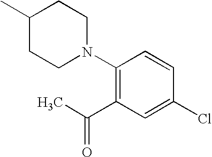 | 84-87° |
| 89 | 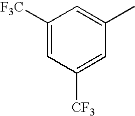 | 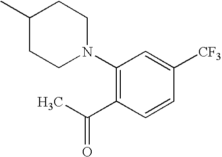 | 158-161° |
| 90 | 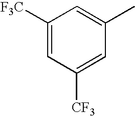 | 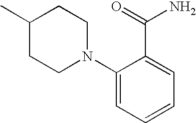 | 95-97° |
| 91 | 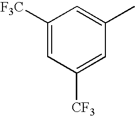 | 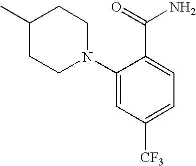 | 1.73-1.86 (m, 2H); 1.94-2.08 (m, 2H);2.30-2.40 (m, 1H); 2.65-2.78 (m, 2H);3.15-3.22 (m, 2H); 6.85 (d, 1H); 7.31(s, 1H); 7.36 (d, 1H); 7.90 (s, 1H); 8.12(d, 1H); 8.43 (s, 2H); 9.08 (d, 1H) |
| 92 | 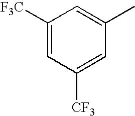 | 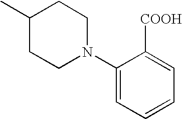 | (DMSO-d₆): 1.53-1.66 (m, 2H);1.89-1.98 (m, 2H); 2.50-2.62 (m, 1H);2.90-3.14 (m, 4H); 7.35-7.40 (m, 2H);7.62 (m, 1H); 7.96 (d, 1H); 8.43 (s,2H); 8.58 (s, 1H) |
| 93 | 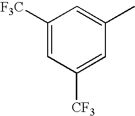 | 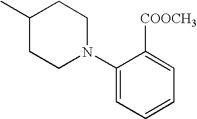 | (DMSO-d₆): 1.55 (dq, 2H); 1.72 (dd,2H); 2.04-2.13 (m, 1H); 2.65 (dt, 2H);3.15 (dt, 2H); 3.78 (s, 3H); 6.95 (t, 1H);7.05 (d, 1H); 7.40 (m, 1H); 7.54 (dd,1H); 8.26 (s, 1H); 8.33 (s, 1H) |
| 94 | 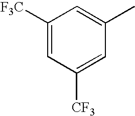 | 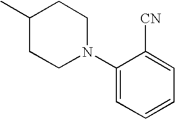 | (DMSO-d₆): 1.40 (dq, 2H); 1.57 (dd,2H); 1.85-1.95 (m, 1H); 2.55 (dt, 2H);3.12-3.22 (m, 2H); 6.81 (t, 1H); 6.90(d, 1H); 7.32 (m, 1H); 7.43 (d, 1H);8.02 (s, 1H); 8.09 (s, 2H) |
| 95 | 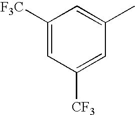 | 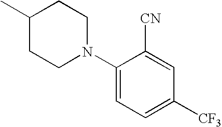 | (DMSO-d₆): 1.57 (dq, 2H); 1.80 (dd,2H); 2.23-2.34 (m, 1H); 2.92 (dt, 2H);3.60 (dt, 2H); 7.22 (d, 1H); 7.79 (dd,1H); 8.03 (d, 1H); 8.33 (s, 3H) |
| 96 | 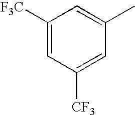 | 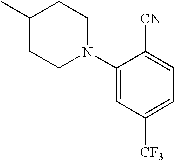 | (DMSO-d₆): 1.52-1.65 (m, 2H);1.73-1.84 (m, 2H); 2.10-2.22 (m, 1H);2.85 (dt, 2H); 3.42-3.53 (m, 2H); 7.30(s, 1H); 7.32 (d, 1H); 7.87 (d, 1H); 8.24(s, 1H); 8.29 (s, 2H) |
| 97 | 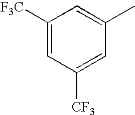 | 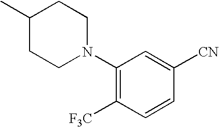 | (DMSO-d₆): 1.51 (dq, 2H), 1.77 (m,2H), 2.29 (m, 1H), 2.74 (t, 2H), 2.93(m, 2H), 7.74 (d, 1H), 7.82 (d, 1H),7.98 (s, 1H), 8.37 (s, 2H), 8.46 (s, 1H). |
| 98 | 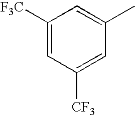 | 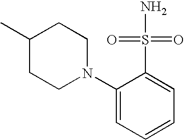 | (DMSO-d₆): 1.62-1.75 (m, 2H);1.78-1.86 (m, 2H); 2.16-2.26 (m, 1H);2.75 (dt, 2H); 3.04-3.13 (m, 2H); 7.37(dd, 1H); 7.52 (d, 1H); 7.64 (dd, 1H);7.88 (d, 1H); 8.32 (s, 1H); 8.38 (s, 2H) |
| 99 | 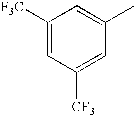 | 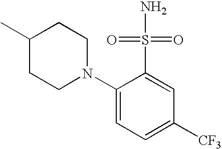 | (DMSO-d₆): 1.51-1.80 (m, 4H), 2.13(m, 1H), 2.71 (m, 1H), 3.12 (d, 1H),7.59 (d, 1H), 7.90 (d, 1H), 8.07 (s, 1H),8.25 (s, 1H), 8.30 (s, 2H). |
| 100 | 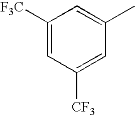 | 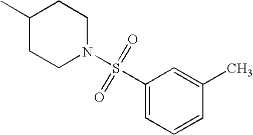 | (DMSO-d₆): 1.42 (m, 2H), 1.76 (m,2H), 2.19-2.33 (m, 3H), 2.48 (s, 3H),3.40-3.50 (m, 2H), 7.47-7.55 (m, 4H),8.38 (s, 2H), 8.56 (s, 2H) |
| 101 | 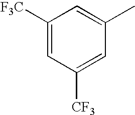 | 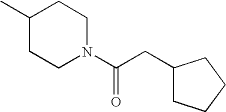 | 111-114° |
| 102 | 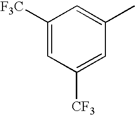 | 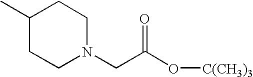 | 115-119° |
| 103 | 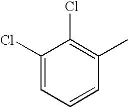 | 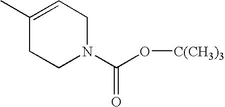 | 163.8, 154.77, 138.30, 136.01, 135.92,132.04, 130.82, 128.04, 80.85, 28.77,24.39 |
| 104 | 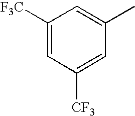 | 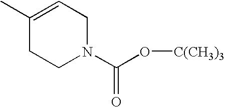 | 141.46, 136.06, 133.38, 133.04, 129.61,128.03, 124.09, 121.37, 80.98, 28.75,24.40 |
| 105 | 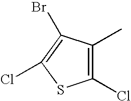 | 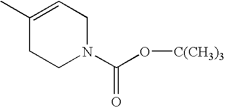 | 164.17, 154.79, 135.90, 130.75, 126.26,108.61, 80.89, 28.78, 24.40 |
| 106 | 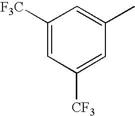 | 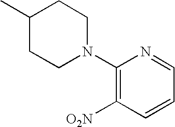 | (DMSO-d₆): 1.47 (dq, 2H); 1.78 (dd,2H); 2.51-2.57 (m, 1H); 2.97 (dt, 2H);3.67 (dt, 2H); 6.88 (dd, 1H); 8.22 (dd,1H); 8.38 (dd, 1H); 8.42 (s, 2H); 8.54(s, 1H) |
| 107 | 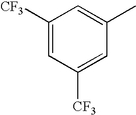 | 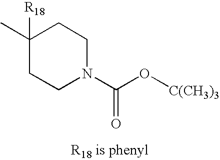 | (DMSO-d₆): δ = 1.10-1.20 (m, 2H);1.32 (s, 9H); 1.59 (m, 2H); 2.42 (broad,1H); 2.98 (m, 2H); 3.70 (m, 2H);6.95-7.06 (m, 3H); 7.16-7.21 (m, 2H);7.75 (s, 1H); 8.10 (s, 2H) |
| 108 | 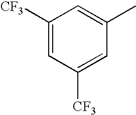 | 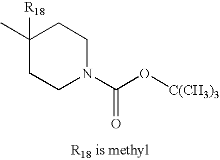 | 131-135° |

Analogously to methods as described in the PROCEDURES (Examples A to Q), but using appropriate starting materials, compounds of formula

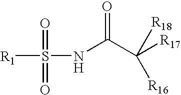

wherein R₁₈ is hydrogen and R₁ and R₁₆+R₁₇ are as defined in TABLE 2 (compounds of formula I, wherein m is 0, n is 0, and R₁ is a group of formula VII) are obtained. If not otherwise indicated in TABLE 2 ¹HNMR and ¹³C-NMR data are determined in CDCl₃.

TABLE 2

| EX | R₁ | R₁₆ + R₁₇ | m.p./¹H-NMR/¹³C-NMR |
|---|---|---|---|
| 109 | 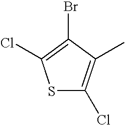 | 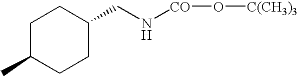 | δ = 0.98(q, 2 H); 1.42(s, 9 H);1.36-2.26(m, 8 H); 2.98(t, 2 H);4.52(broad, 1 H) |
| 110 | 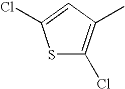 | 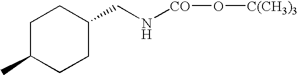 | 0.94(dq, 2 H), 1.33-1.49(m,12 H), 1.83(broad d, 2 H); 1.91(broad d, 2 H), 2.14(tt, 1 H), 2.95(d, 2 H), 7.28(s, 1 H) |
| 111 | 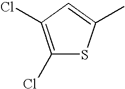 | 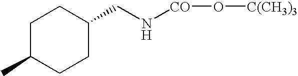 | 0.92(dq, 2 H), 1.32-1.48(m,12 H), 1.65(broad, 1 H), 1.82(d,2 H), 1.88(d, 2 H), 2.09(tt, 1 H),2.93(d, 2 H), 7.61(s, 1 H) |
| 112 | 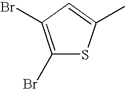 | 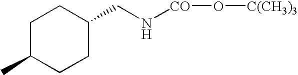 | 0.93(dq, 2 H), 1.35-1.50(m,11 H), 1.76-2.05(m, 5 H), 2.10(tt, 1 H), 2.95(d, 2 H), 4.72(broad, 1 H), 7.63(s, 1 H) |
| 113 | 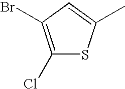 | 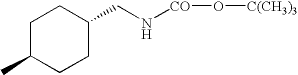 | 0.94(dq, 2 H), 1.35-1.49(m,12 H), 1.78-1.93(m, 4 H), 2.11(tt, 1 H), 2.94(d, 2 H), 4.78(broad, 1 H), 7.65(s, 1 H) |
| 114 | 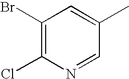 | 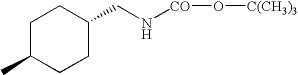 | 0.92(dq, 2 H), 1.31-1.46(m, 12 H),1.83(broad t, 2 H), 2.03-2.14(m,3 H), 2.93(d, 2 H), 4.72(broad,1 H), 8.58(d, 1 H), 8.87(d, 1 H) |
| 115 | 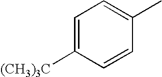 | 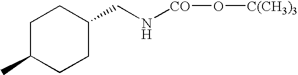 | 0.90(m, 2 H); 1.30(m, 1 H);1.38(s, 9 H); 1.42(s, 9 H); 1.75-2.20(m, 7 H); 2.98(t, 2 H);4.52(broad, 1 H); 7.55(d, 2 H);7.92(d, 2 H); 8.30(s, 1 H) |
| 116 | 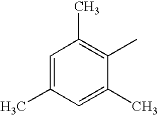 | 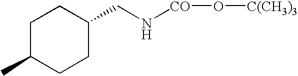 | 0.92(q, 2 H); 1.41(s, 9 H); 1.25-2.18(m, 8 H); 2.35(s, 3 H); 2.70(s, 6 H); 2.98(t, 2 H); 4.50(broad,1 H); 6.94(s, 2 H); 8.52(s, 1 H) |
| 117 | 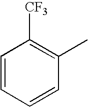 | 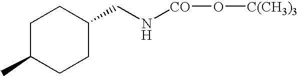 | 0.92(q, 2 H); 1.42(s, 9 H); 1.20-2.18(m, 8 H); 2.94(t, 2 H); 4.58(broad, 1 H); 7.78(t, 2 H); 7.86(m, 1 H); 8.41(s, 1 H); 8.50(dd, 1 H) |
| 118 | 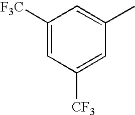 | 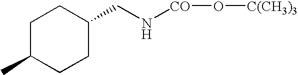 | 0.95(m, 2 H); 1.20-2.30(m, 8 H);1.46(s, 9 H); 3.00(t, 2 H); 4.58broad, 1 H); 8.06(s, 1 H); 8.50(s, 2 H) |
| 119 | 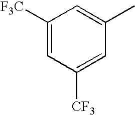 | 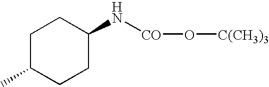 | 1.02(q, 2 H); 1.39(s, 9 H); 1.40-1.46(m, 1 H); 1.72-1.88(m, 5 H); 2.08(t, 1 H); 3.30(broad, 1 H); 4.48(d,1 H); 7.90(s, 1 H); 8.35(s, 2 H) |
| 120 | 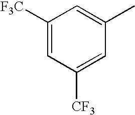 | 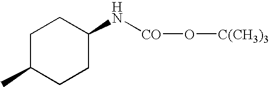 | 1.40(s, 9 H); 1.40-1.80(m, 8 H);2.25(m, 1 H); 3.55(m, 1 H); 7.92(s, 1 H); 8.36(s, 2 H) |
| 121 | 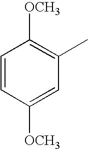 | 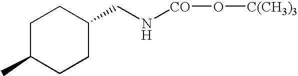 | 1.00(m, 2 H); 1.30-2.00(m, 7 H);1.42(s, 9 H); 2.20(t, 1 H); .98(t, 2 H);3.80(s, 3 H); 3.90(s, 3 H); 5.58(broad, 1 H); 6.95(d, 1 H); 7.14(dd,1 H); 7.58(d, 1 H); 8.50(s, 1 H) |
| 122 | 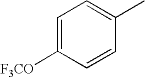 | 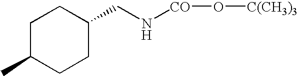 | 0.98(q, 2 H); 1.41(s, 9 H); 1.36-2.20(m, 8 H); 2.98(t, 2 H); 4.55(broad, 1 H); 7.30 and 8.10(2 d,4 H); 8.13(s, 1 H) |
| 123 | 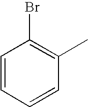 | 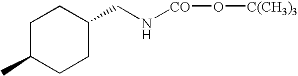 | 0.95(q, 2 H); 1.43(s, 9 H); 1.20-2.26(m, 8 H); 2.95(t, 2 H); 4.53(broad, 1 H); 7.40-7.55(m, 2 H);7.70 and 8.30(2 dd, 2 H); 8.46(s, 1 H) |
| 124 | 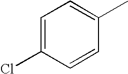 | 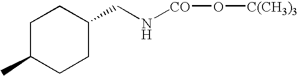 | 0.91(q, 2 H); 1.40(s, 9 H); 1.25-1.63(m, 3 H); 1.78-2.18(m, 5 H);2.96(t, 2 H); 4.58(broad, 1 H); 7.50and 7.98(AB, 2 H); 8.38(s, 1 H) |
| 125 | 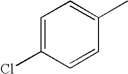 | 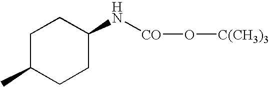 | 1.42(s, 9 H); 1.54-1.78(m, 8 H);2.30(m, 1 H); 3.64(m, 1 H); 4.50(broad, 1 H); 7.51 and 7.99(AB,4 H); 8.36(broad, 1 H) |
| 126 | 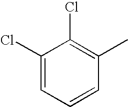 | 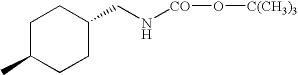 | 1.00(m, 2 H); 1.30-2.00(m, 7 H);1.42(s, 9 H); 2.20(t, 1 H); 2.98(t, 2 H); 5.58(broad, 1 H); 7.40(t, 1 H); 7.70(d, 1 H); 8.22(d, 1 H) |
| 127 | 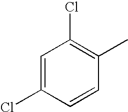 | 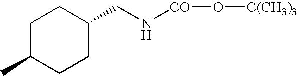 | 0.98(q, 2 H); 1.41(s, 9 H); 1.55-2.22(m, 8 H); 2.85(t, 2 H); 4.54(broad, 1 H); 7.42(dd, 1 H); 7.52(d, 1 H); 8.19(d, 1 H) |
| 128 | 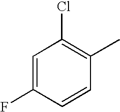 | 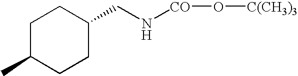 | 0.98(q, 2 H); 1.40(s, 9 H); 1.25-2.25(m, 8 H); 2.98(t, 2 H); 4.70(broad, 1 H); 7.13-7.24(m, 2 H);8.26(dd, 1 H); 8.58(s, 1 H) |
| 129 | 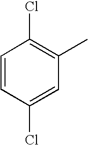 | 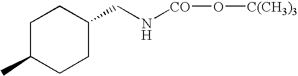 | 0.80-2.00(m, 9 H); 1.42(s, 9 H);2.20(t, 1 H); 2.98(t, 1 H); 4.55(broad, 1 H); 7.36-7.50(m, 2 H);8.20(m, 2 H) |
| 130 | 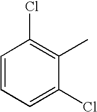 | 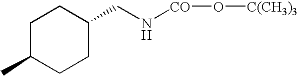 | 0.98(q, 2 H); 1.43(s, 9 H); 1.22-2.30(m, 8 H); 2.98(t, 2 H); 4.58(broad, 1 H); 7.30-7.58(m, 3 H) |
| 131 | 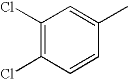 | 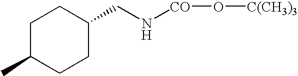 | 0.98(q, 2 H); 1.41(s, 9 H); 1.35-2.20(m, 8 H); 2.98(t, 2 H); 4.52(broad, 1 H); 7.60(d, 1 H); 7.70(dd, 1 H); 8.10(d, 1 H) |
| 132 | 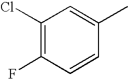 | 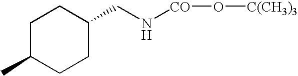 | 0.94(q, 2 H); 1.40(s, 9 H);1.25-1.41(m, 2 H); 1.70-1.96(m, 5 H); 2.10(t, 1 H); 2.94(t,2 H); 4.58(broad, 1 H); 7.30(m, 1 H); 7.96(m, 1 H); 8.12(m, 1 H); 8.39(s, 1 H) |
| 133 | 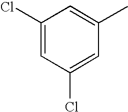 | 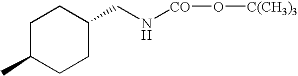 | 0.91(q, 2 H); 1.40(s, 9 H); 1.26-1.70(m, 3 H); 1.78-2.20(m, 5 H); 2.95(t, 2 H); 4.52(broad, 1 H); 7.54(m,1 H); 7.86(m, 2 H); 8.50(s, 1 H) |
| 134 | 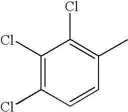 | 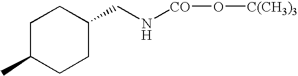 | 0.98(q, 2 H); 1.42(s, 9 H); 1.38-2.30(m, 8 H); 2.96(t, 2 H); 4.54(broad,1 H); 7.60(d, 1 H); 8.08(d, 1 H) |
| 135 | 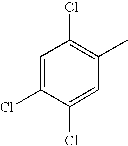 | 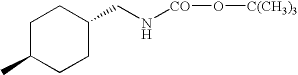 | (CDCl₃ + 10% DMSO-d₆) 0.98(q, 2 H); 1.42(s, 9 H); 1.25-2.25(m,8 H); 2.95(d, 2 H); 5.10(broad,1 H); 7.60(s, 1 H); 8.24(s, 1 H) |
| 136 | 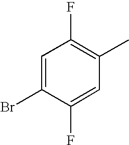 | 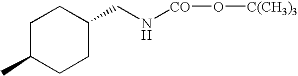 | 0.58-1.04(m, 2 H); 1.42(s, 9 H);1.30-1.96(m, 7 H); 2.16(m, 1 H);2.98(t, 2 H); 4.58(broad, 1 H); 7.48(dd, 1 H); 7.82(dd, 1 H); 8.65(s, 1 H) |
| 137 | 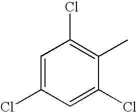 | 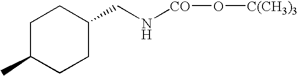 | 0.92(q, 2 H); 1.42(s, 9 H); 1.20-1.54(m, 2 H); 1.70-2.20(m, 6 H);2.90(d, 2 H); 7.42(s, 2 H) |
| 138 | 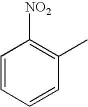 | 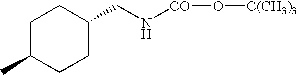 | 0.90(m, 2 H); 1.20-2.30(m, 8 H);1.46(s, 9 H); 2.98(t, 2 H); 4.58(broad, 1 H); 7.75-7.82(m, 3 H);8.41(m, 1 H) |
| 139 | 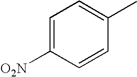 | 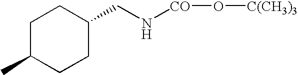 | 0.94(q, 2 H); 1.42(s, 9 H); 1.20-1.45(m, 1 H); 1.60-2.20(m, 7 H);2.95(t, 2 H); 4.58(broad, 1 H); 8.23and 8.38(AB, 4 H), 8.60(s, 1 H) |
| 140 | 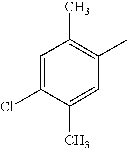 | 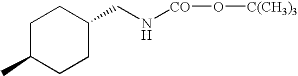 | (m, 2 H); 1.30-2.00(m, 7 H); 1.42(s, 9 H); 2.20(t, 1 H); 2.40(s,3 H); 2.60(s, 3 H); 2.98(t, 2 H);5.58(broad, 1 H); 7.40(t, 1 H);7.70(d, 1 H); 8.22(d, 1 H) |
| 141 | 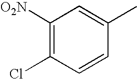 | 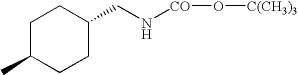 | 0.94(q, 2 H); 1.41(s, 9 H); 1.24-1.70(m, 2 H); 1.80-2.20(m, 6 H);2.98(q, 2 H); 4.58(broad, 1 H);7.75(d, 1 H); 8.22(dd, 1 H); 8.46(d, 1 H); 8.54(s, 1 H) |
| 142 | 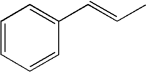 | 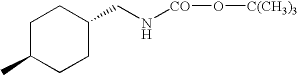 | 0.93(q, 2 H); 1.40(s, 9 H); 1.32-1.58(m, 2 H); 1.78-2.20(m, 6 H);2.92(d, 2 H); 7.04 and 7.62(AB,2 H); 7.34-7.56(m, 5 H) |
| 143 | 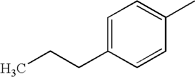 | 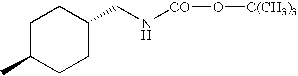 | 0.95(m, 4 H); 1.30-2.20(m, 10 H);1.42(s, 9 H); 2.70(t, 2 H); 2.98(t,2 H); 4.56(broad, 1 H); 7.30(d,2 H); 7.90(d, 2 H); 8.18(s, 1 H) |
| 144 | 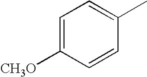 | 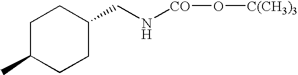 | 0.90(m, 2 H); 1.20-2.20(m,8 H); 1.48(s, 9 H); 2.98(t,2 H); 3.90(s, 3 H); 4.55(broad, 1 H); 6.99(d, 2 H);8.00(d, 2 H); 8.20(s, 1 H) |
| 145 | 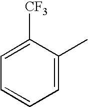 | 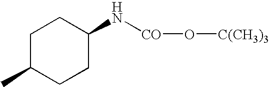 | CDCl₃ + 5% DMSO-d₆: 1.43(s, 9 H), 1.54-1.73(m, 4 H);2.32(m, 1 H); 2.52-2.64(m, 4 H);3.76(m, 1 H); 5.32(bd, 1 H); 7.72-7.78(m, 2 H); 7.84-7.88(m, 1 H);8.45-8.50(m, 1 H) |
| 146 | 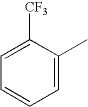 | 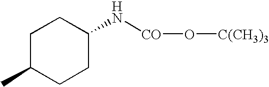 | CDCl₃ + 5% CD₃OD: 1.06(m,2 H); 1.40(s, 9 H); 1.43(m, 2 H);1.84(m, 2 H); 2.03(m, 2 H); 2.08(m, 1 H); 3.30(broad, 1 H); 7.71-7.77(m, 2 H); 7.82-7.87(m, 1 H);8.46-8.51(m, 1 H) |
| 147 | 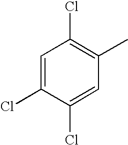 | 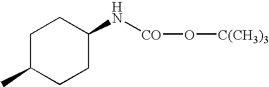 | CDCl₃ + 5% DMSO-d₆: 1.42(s,9 H); 1.55(m, 2 H); 1.60-1.80(m,5 H); 2.38(m, 1 H); 2.50(m, 2 H);3.75(m, 1 H); 5.30(bd, 1 H);7.70(s, 1 H); 8.30(s, 1 H) |
| 148 | 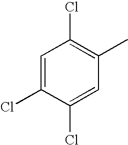 | 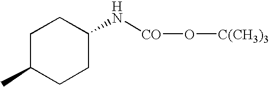 | CDCl₃ + 5% CD₃OD: 1.08(m,2 H); 1.42(s, 9 H); 1.47(m, 2 H);1.88(m, 2 H); 2.03(m, 2 H); 2.12(m, 1 H); 2.31(broad, 1 H); 7.59(s, 1 H); 8.31(s, 1 H) |
| 149 | 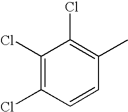 | 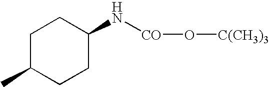 | CDCl₃ + 5% DMSO-d₆: 1.45(s,9 H); 1.50(m, 2 H); 1.55-1.75(m,4 H); 2.32(m, 1 H); 2.58(m, 2 H);3.77(m, 1 H); 5.33(bd, 1 H);7.61(d, 1 H); 8.13(d, 1 H) |
| 150 | 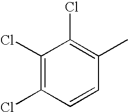 | 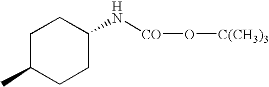 | CDCl₃ + 5% CD₃OD: 1.08(m,2 H); 1.40(s, 9 H); 1.44(m, 2 H);1.86(m, 2 H); 2.02(m, 2 H); 2.10(m, 1 H); 3.28(m, 1 H); 7.55(d,1 H); 8.11(m, 1 H) |
| 151 | 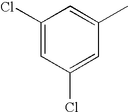 | 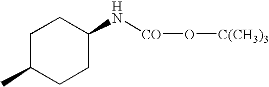 | CDCl₃ + 5% DMSO-d₆: 1.40(s,9 H); 1.50-1.78(m, 6 H); 2.32(m,1 H); 2.54(m, 2 H); 3.73(m, 1 H);5.22(bd, 1 H); 7.60(s, 1 H); 7.90(s, 1 H) |
| 152 | 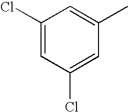 | 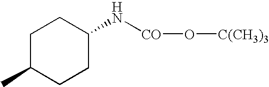 | CDCl₃ + 5% CD₃OD: 1.08(m, 2 H);1.40(s, 9 H); 1.47(m, 2 H); 1.85(m, 2 H); 2.04(m, 1 H); 3.29(broad,1 H); 7.56(t, 1 H); 7.87(d, 1 H) |
| 153 | 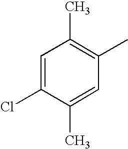 | 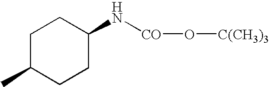 | CDCl₃ + 5% DMSO-d₆: 1.42(s,9 H); 1.70-1.80(m, 8 H); 2.30(m,1 H); 2.40(s, 3 H); 2.56(s, 3 H);3.77(m, 1 H); 5.25(bd, 1 H);7.24(s, 1 H); 7.98(s, 1 H) |
| 154 | 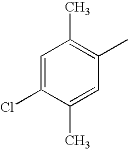 | 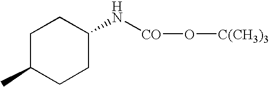 | CDCl₃ + 5% CD₃OD: 1.05(m,2 H); 1.38(s, 9 H); 1.42(m, 2 H);1.80(m, 2 H); 1.97(m, 2 H); 2.07(m, 1 H); 2.35(s, 3 H); 2.50(s,3 H); 3.25(broad, 1 H); 7.22(s,1 H); 7.95(s, 1 H) |
| 155 | 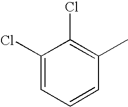 | 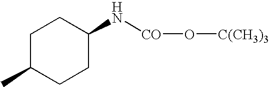 | CDCl₃ + 5% DMSO-d₆: 1.44(s,9 H); 1.54(m, 2 H); 1.62-1.79(m,4 H); 2.33-2.44(m, 5 H); 3.77(broad, 1 H); 5.28(bd, 1 H); 7.41(t,1 H); 7.71(dd, 1 H); 8.20(dd, 1 H) |
| 156 | 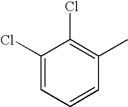 | 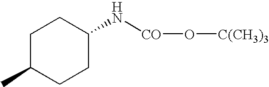 | CDCl₃ + 5% CD₃OD: 1.08(m, 2 H);1.40(s, 9 H); 1.44(m, 2 H); 1.86(m,2 H); 2.01(m, 2 H); 2.12(m, 1 H);3.28(broad, 1 H); 7.38(t, 1 H);7.68(dd, 1 H); 8.18(dd, 1 H) |
| 157 | 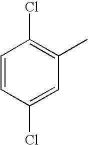 | 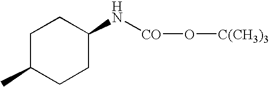 | CDCl₃ + 5% DMSO-d₆: 1.42(s,9 H); 1.55(m, 2 H); 1.60-1.77(m,4 H); 2.35(m, 2 H); 3.76(m, 1 H);5.24(m, 1 H); 7.43(d, 1 H); 7.50(dd, 1 H); 8.24(d, 1 H) |
| 158 | 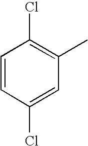 | 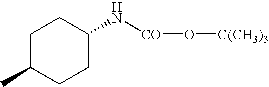 | CDCl₃ + 5% CD₃OD: 1.08(m,2 H); 1.41(m, 9 H); 1.46(m, 2 H);1.88(m, 2 H); 2.03(m, 2 H); 2.13(m, 1 H); 3.28(broad, 1 H); 7.39(d, 1 H); 7.48(dd, 1 H); 8.20(d, 1 H) |
| 159 | 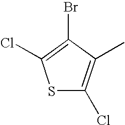 | 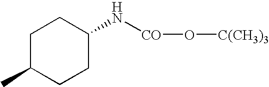 | 1.09(dq, 2 H), 1.41(s, 9 H), 1.52(dq, 2 H), 1.92(broad d, 2 H),2.05(broad, d, 2 H), 2.15(tt,1 H), 3.32(broad, 1 H)trans isomer |
| 160 | 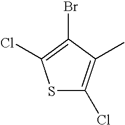 | 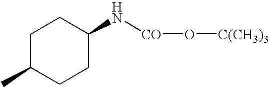 | (CDCl₃ + 5% DMSO-d₆): 23.814,28.811, 29.586, 29.944, 44.056,45.056, 79.296, 108.900,125.462, 155.603, 175.574 |
| 161 | 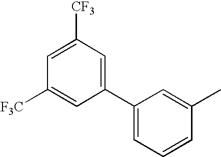 | 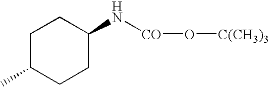 | 223-225° |
| 162 | 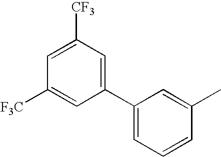 | 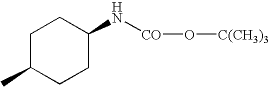 | (DMSO-d₆): 8.30(s, 2 H), 8.15(s, 2 H), 8.07(d, J = 7.82.16(br.s, 1 H), Hz, 1 H), 7.92(d, J =7.8 Hz, 1 H), 7.68(t, J = 7.8 Hz,1 H), 1.35-1.73(m, 8 H), 1.35(s,9 H) |
| 163 | 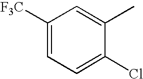 | 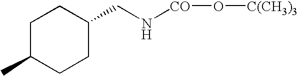 | DMSO-d₆: 0.77(m, 2 H); 1.08(m, 2 H); 1.10(m, 1 H); 1.32(s,9 H); 1.62(m, 2 H); 1.72(m, 2 H);2.20(m, 1 H); 2.70(t, 2 H); 6.71(t, 1 H); 7.91(d, 1 H); 8.07(dd,1 H); 8.22(d, 1 H); 12.65(bs, 1 H) |
| 164 | 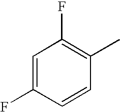 | 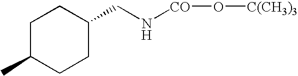 | 0.94(m, 2 H); 1.32-1.50(m, 3 H);1.43(s, 9 H); 1.83(m, 2 H); 1.91(m, 2 H); 2.14(m, 1 H); 2.97(t,2 H); 4.54(broad, 1 H); 6.95(m,1 H); 7.06(m, m, 1 H); 8.11(m,1 H); 8.68(bs, 1 H) |
| 165 | 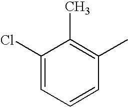 | 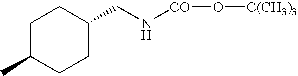 | 1.04(m, 2 H); 1.32-1.50(m, 3 H);1.42(s, 9 H); 1.82(m, 2 H); 1.89(m, 2 H); 2.16(m, 1 H); 2.68(s,3 H); 2.96(t, 2 H); 4.55(broad,1 H); 7.33(t, 1 H); 7.64(dd, 1 H);8.13(dd, 1 H); 8.77(bs, 1 H) |
| 166 | 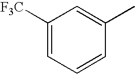 | 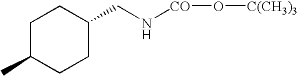 | 0.92(m, 2 H); 1.32-1.50(m, 3 H);1.43(s, 9 H); 1.82(m, 2 H); 1.84(m, 2 H), 2.12(m, 1 H); 2.96(t,2 H); 4.55(broad, 1 H); 7.70(t,1 H); 7.89(d, 1 H); 8.28(s, 1 H);8.31(s, 1 H); 8.63(bs, 1 H) |
| 167 | 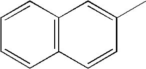 | 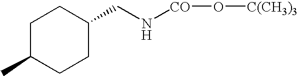 | 0.88(m, 2 H); 1.25-1.48(m, 3 H);1.43(s, 9 H); 1.81(m, 4 H); 2.10(m,1 H); 2.92(t, 2 H); 4.70(t, 1 H); 7.57-7.69(m, 3 H); 7.92(d, 1 H); 7.96(s,2 H); 8.01(d, 1 H); 8.63(s, 1 H) |
| 168 | 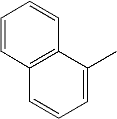 | 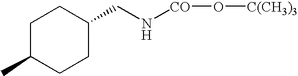 | 0.83(m, 2 H); 1.22(m, 2 H); 1.28(m, 1 H); 1.42(s, 9 H); 1.72(m,4 H); 2.08(m, 1 H); 2.90(t, 2 H);4.49(broad, 1 H); 7.58-7.69(m,3 H); 7.98(d, 1 H); 8.13(d, 1 H); 8.52(dd, 1 H); 8.59(d, 1 H); 9.03 bs, 1 H) |
| 169 | 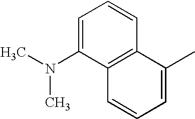 | 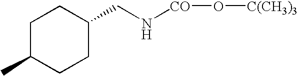 | 0.83(m, 2 H); 1.17-1.36(m, 3 H);1.46(s, 9 H); 1.74(t, 4 H); 2.10(m, 1 H); 2.80-3.00(m, 2 H); 2.94(s, 6 H); 4.52(broad, 1 H); 7.23(d, 1 H); 7.53-7.64(m, 2 H); 8.27(d, 1 H); 8.50(dd, 1 H); 8.61(d,1 H); 9.15 bs, 1 H) |
| 170 | 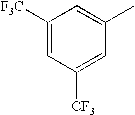 | 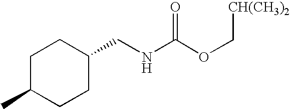 | 165-169° |
| 171 | 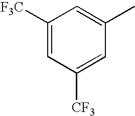 | 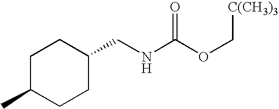 | 90-94° |
| 172 | 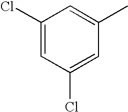 | 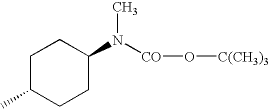 | (DMSO-d₆): 8.07(t, J = 1.9 Hz,1 H), 7.86(d, J = 1.9 Hz, 2 H),3.70(br.s, 1 H), 2.64(s, 3 H),2.20(tt, J = 3.3 + 8.6 Hz, 1 H),1.23-1.64(m, 8 H), 1.38(s, 9 H) |
| 173 | 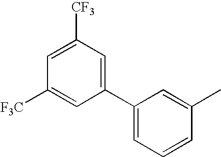 | 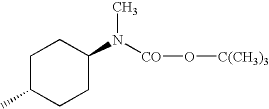 | (DMSO-d₆): 12.16(s, 1 H), 8.37(s, 2 H), 8.20-8.25(m, 37.99-8.03(m, 1 H), 7.81(t, J = 7.9Hz, 1 H), 3.69(br.s, 1 H), 2.63(s,3 H), 2.19(tt, J = 3.4 + 12 Hz,1 H), 1.77-1.85(m, 2 H), 1.21-1.63(m, 6 H), 1.37(s, 9 H) |
| 174 | 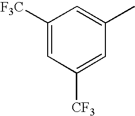 | 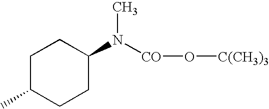 | (DMSO-d₆): 8.22(s, 2 H), 8.15(s,1 H), 3.45-3.70(br.m, 1 H),2.60(s, 3 H), 1.69-1.84(m,3 H), 1.36(s, 9 H), 1.12-1.57(m, 6 H) |
| 175 | 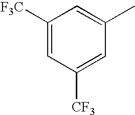 | 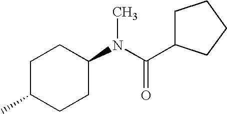 | (DMSO-d₆): 2 rotamers,selected signals: 12.47(br.s,1 H), 8.59(s, 1 H), 8.42(s, 2 H),4.12 + 3.66(2 × m, 1 H), 2.79 +2.62(2 × s, 3 H) |
| 176 | 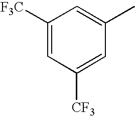 | 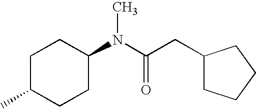 | (DMSO-d₆): 2 rotamersselected signals: 12.41(br.s,1 H), 8.59(s, 1 H), 8.42(s, 2 H),4.10-4.19(m, 1 H), 2.74 + 2.61(2 × s, 3 H) |
| 177 | 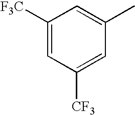 | 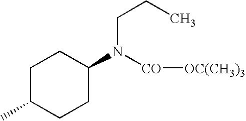 | 12.47(s, 1 H), 8.60(s, 1 H), 8.43(s, 2 H), 3.57(br.s, 1 H), 2.96(br.s, 2 H), 2.19(tt, J = 3.4 + 12Hz, 1 H), 1.18-1.82(m, 10 H),1.37(s, 9 H), 0.80(t, J = 7 Hz, 3 H) |
| 178 | 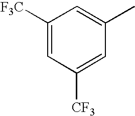 | 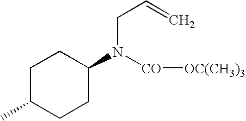 | (DMSO-d₆): 12.47(s, 1 H),8.59(s, 1 H), 8.42(s, 2 H), 5.68-5.78(m, 1 H), 5.09(d, J = 17.7 Hz,1 H), 5.04(d, J = 9 HZ, 1 H), 3.68(br.s, 3 H), 2.17(tt, J = 3.2 + 9Hz, 1 H), 1.16-1.67(m, 8 H),1.37(s, 9 H) |
| 179 | 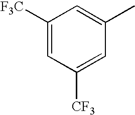 | 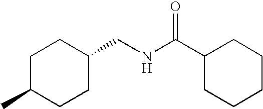 | 198-204° |
| 180 | 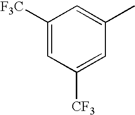 | 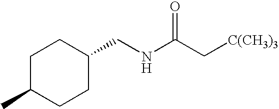 | 136-140° |
| 181 | 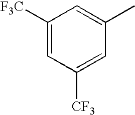 | 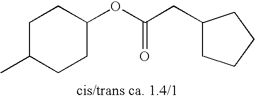 | (DMSO-d₆): E/Z stereoisomers,selected signals: 12.5 (br.s,1 H), 8.59(s, 1 H), 8.41(s, 2 H),4.81 + 4.51(br.s + m, 1 H) |
| 182 | 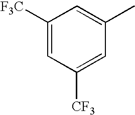 | 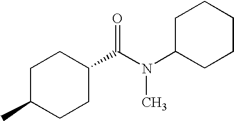 | 230-238° |
| 183 | 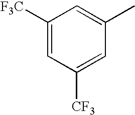 | 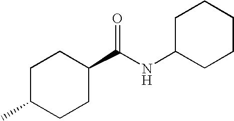 | 220-230° |
| 184 | 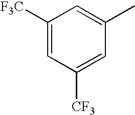 | 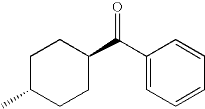 | 173-175° |
| 185 | 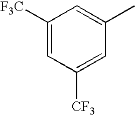 | 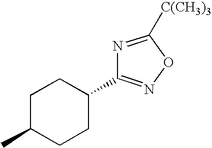 | (DMSO-d₆): 1.54(s, 9 H), 1.55-1.77(m, 4 H), 2.10(dd, 2 H),2.31(dd, 2 H), 2.57(tt, 1 H), 3.19(tt, 1 H), 8.68(s, 2 H), 8.85(s, 1) |

Analogously to methods as described in the PROCEDURES (Examples A to Q), but using appropriate starting materials, compounds of formula

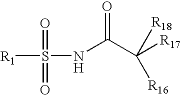

wherein R₁₈ is hydrogen and R₁ and R₁₆+R₁₇ are as defined in TABLE 3 (compounds of formula I, wherein m is 0, n is 0, and R₁ is a group of formula VII) are obtained. If not otherwise indicated in TABLE 3 ¹³C-NMR and ¹HNMR data are determined in CDCl₃.

TABLE 3

| EX | R₁ | R₁₆ + R₁₇ | m.p./¹HNMR/¹³C-NMR |
|---|---|---|---|
| 186 | 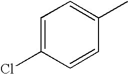 | 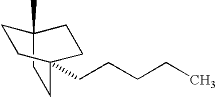 | (DMSO-d₆): δ = 0.80-0.95(m,3 H); 0.95-1.40(m, 10 H); 1.50-1.75(m, 8 H); 7.62/7.82(AB, 4 H) |
| 187 | 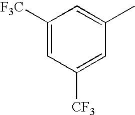 | 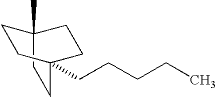 | 322-333° |
| 188 | 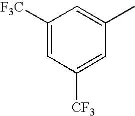 | 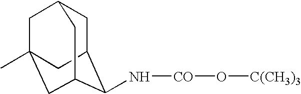 | 98-100°/(DMSO-d₆): 1.38/1.40(s, 9 H), 1.60-2.10(m, 12 H); 3.41-3.57(m, 1 H); 6.68/6.80(bd,1 H); 8.36/8.40(s, 2 H); 8.48/8.50(s, 1 H) |
| 189 | 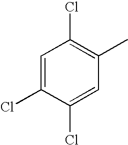 | 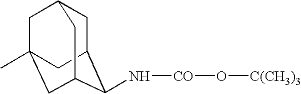 | 1.47(s, 9 H); 1.51-2.13(m,12 H); 3.72(m, 1 H); 4.81(d,1 H); 7.60(s, 1 H); 8.30(s, 1 H) |
| 190 | 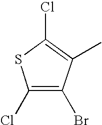 | 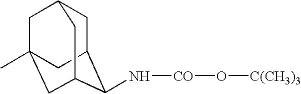 | 132-133° |
| 191 | 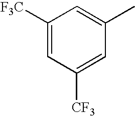 | 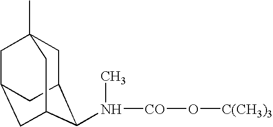 | 2 rotamers, selected signals:8.55(s, 2 H), 8.35 + 8.32(2 × br,s, 1 H), 8.16(s, 1 H), 3.87 + 3.82 × s, 1 H), 3.05 + 3.00(2 × s, 3 H);2.40 + 2.32(2 × s, 2 H), 1.47(s, 9 H) |
| 192 | 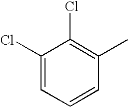 | 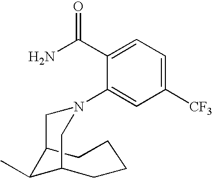 | (DMSO-d₆): 173.12, 170.12,150.43, 136.52, 135.24, 133.86,131.29, 130.04, 129.79, 129.19,128.87, 128.47, 125.10, 122.94,117.86, 115.85, 60.09, 47.76,32.80, 31.60, 26.06 |
| 193 | 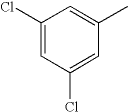 | 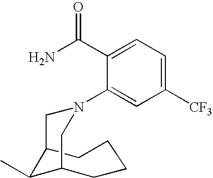 | (DMSO-d₆): 170.59, 150.93,136.92, 135.02, 134.99, 130.44,130.20, 129.63, 126.47, 125.56,118.24, 116.16, 60.62, 48.20,33.27, 32.02, 26.55 |
| 194 | 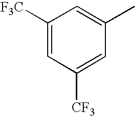 | 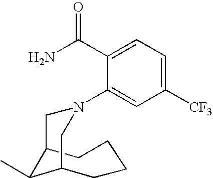 | (CDCl₃/DMSO-d₆): 173.42,170.56, 151.37, 142.90, 134.67132.89, 132.61, 130.16, 129.31,128.97, 128.51, 126.99, 119.30,116.91, 61.47, 48.66, 33.60,32.09, 26.78 |
| 195 | 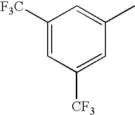 | 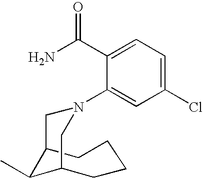 | (CDCl₃/DMSO-d₆): 173.72,170.83, 152.28, 143.07, 136.26,132.79, 132.52, 130.78, 130.13,128.95, 127.04, 122.48, 121.83,120.56, 61.41, 48.74, 33.65,32.13, 26.82 |
| 196 | 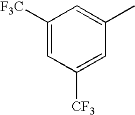 | 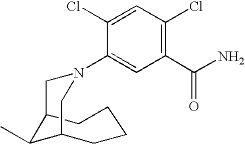 | 173.14, 167.61, 149.63, 142.55,133.06, 132.70, 132.46, 132.37,129.13, 127.26, 124.99, 124.20,123.54, 60.42, 48.87, 40.38,33.78, 32.27, 27.00 |
| 197 | 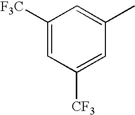 | 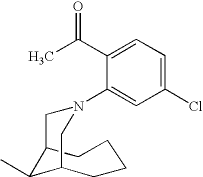 | 171.33, 141.88, 133.33, 133.06,129.38, 127.69, 123.86, 62.30,33.47, 31.79, 26.45 |
| 198 | 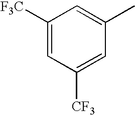 | 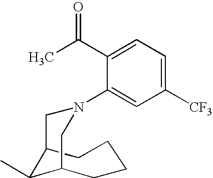 | 203.85, 171.03, 150.68, 141.52,133.44, 133.17, 129.45, 128.13,127.90, 119.82, 118.09, 61.87,48.42, 33.89, 32.13, 31.92,26.61 |
| 199 | 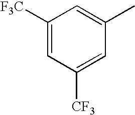 | 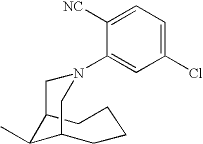 | 170.40, 154.09, 140.96, 138.32,134.78, 133.31, 133.04, 132.76,132.48, 129.06, 129.03, 127.61,125.55, 123.38, 121.20, 117.12,115.07, 112.97, 101.03, 55.60,48.45, 33.07, 32.28, 26.09 |
| 200 | 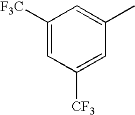 | 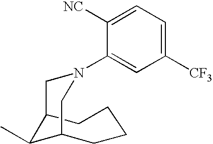 | 170.68, 155.72, 141.43, 136.12,135.92, 133.49, 133.21, 132.93,129.47, 127.98, 123.81, 121.63,118.99, 118.97, 117.80, 116.45,116.42, 109.19, 60.26, 48.41,33.75, 32.02, 26.87 |
| 201 | 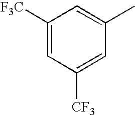 | 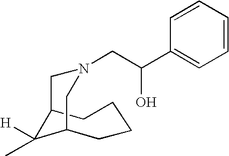 | 95-98° |
| 202 | 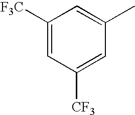 | 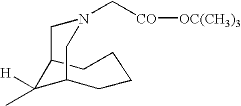 | 170.44, 132.95, 132.68, 132.41,132.13, 127.36, 126.23, 125.66,124.07, 121.89, 119.71, 81.09,62.52, 61.35, 50.29, 33.16,28.43, 27.16 |
| 203 | 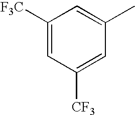 | 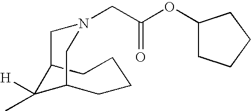 | 8.53(s, 2 H), 8.11(s, 2 H), 5.25(m, 1 H), 3.56(s, 2 H), 3.13(bd,2 H), 2.98(bs, 1 H), 2.88(bs,1 H), 2.67(bs, 2 H), 2.21(s, 1 H),2.02(m, 2 H), 1.83(m, 2 H),1.78-1.58(m, 10 H), 1.40(m, 2 H) |
| 204 | 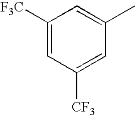 | 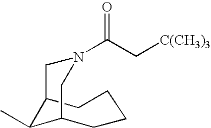 | 173.01, 171.44, 142.73, 133.31,133.03, 132.76, 132.49, 129.07,129.05, 127.18, 127.15, 127.12,126.11, 123.93, 121.76, 119.59,54.84, 49.82, 48.82, 45.09,33.42, 32.25, 30.38, 27.01,26.24 |
| 205 | 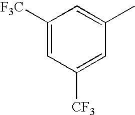 | 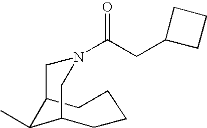 | 173.15, 142.63, 132.91, 132.56,132.22, 128.98, 127.10, 124.14,121.42, 53.68, 49.63, 48.88,33.08, 32.61, 32.28, 28.89,26.96, 26.25, 19.02 |
| 206 | 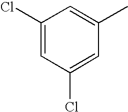 | 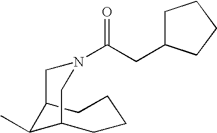 | 172.71, 171.25, 141.71, 136.26,134.33, 127.18, 127.11, 53.92,49.49, 49.15, 39.74, 36.96,33.50, 33.18, 32.72, 32.32,32.11, 26.99, 26.20, 25.34 |
| 207 | 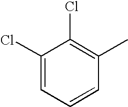 | 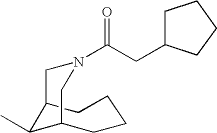 | 172.45, 171.36, 138.44, 135.99,135.85, 132.09, 130.50, 128.07,53.80, 49.61, 49.18, 39.74,36.89, 33.63, 33.24, 33.19,32.08, 27.01, 26.32, 25.34 |
| 208 | 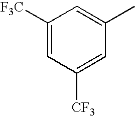 | 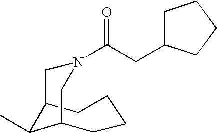 | 173.15, 171.18, 141.69, 133.40,133.12, 132.85, 129.35, 127.82,123.82, 121.64, 54.00, 49.41,49.25, 39.67, 37.02, 33.53,33.21, 33.14, 32.26, 32.04,26.92, 26.13, 25.29 |
| 209 | 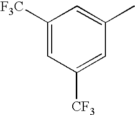 | 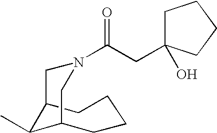 | 172.78, 172.45, 142.56, 133.41,133.07, 132.72, 132.38, 129.10,127.25, 124.18, 121.46, 80.1953.66, 49.62, 48.74, 42.62,33.21, 33.08, 32.37, 32.26,30.05, 27.02, 26.21, 24.28,24.18 |
| 210 | 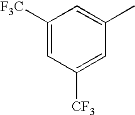 | 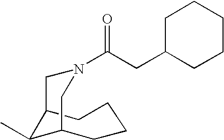 | 172.78, 171.30, 141.74, 133.38,133.10, 129.37, 127.78, 123.83,54.12, 49.46, 49.24, 41.21,35.46, 33.82, 33.54, 33.23,32.29, 32.01, 26.92, 26.54,26.49, 26.12 |
| 211 | 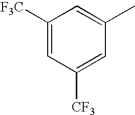 | 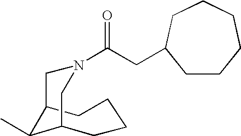 | 173.83, 171.03, 141.51, 133.77,133.42, 133.08, 132.73, 129.39,127.91, 126.77, 124.06, 121.34,118.82, 54.21, 49.48, 49.22,41.58, 37.19, 35.15, 35.08, 33.48,33.13, 32.19, 31.93, 28.533,26.89, 26.51, 26.44, 26.07 |
| 212 | 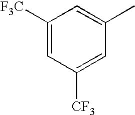 | 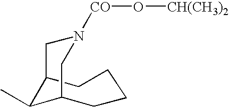 | 171.16, 155.61, 141.55, 133.42,133.14, 129.39, 127.87, 123.81,69.31, 49.48, 33.34, 32.03,26.60, 22.61 |
| 213 | 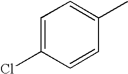 | 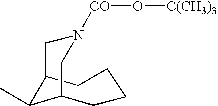 | 130.45, 130.21, 129.74, 129.65,80.35, 49.41, 32.09, 28.86 mix |
| 214 | 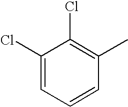 | 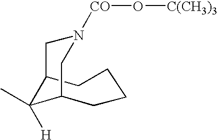 | 171.46, 155.14, 138.41, 135.99,135.85, 132.10, 130.49, 128.07,80.40, 49.65, 33.28, 32.01,28.86, 26.67 |
| 215 | 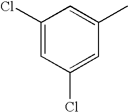 | 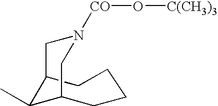 | 171.26, 155.28, 141.51, 136.30,134.42, 127.21, 127.04, 80.69,49.49, 33.21, 32.08, 28.86,26.58 |
| 216 | 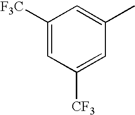 | 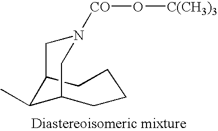 | Diastereoisomeric mixture ofcompounds of Example 217 andExample 218 |
| 217 | 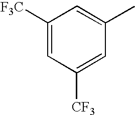 | 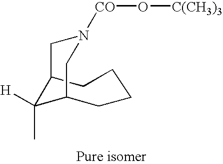 | 170.84, 154.71, 141.06, 133.27,132.99, 132.99, 132.72, 132.44,129.03, 129.00, 127.54, 127.52,123.39, 121.22, 80.07, 49.04,32.83, 31.66, 28.45, 26.15 |
| 218 | 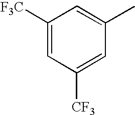 | 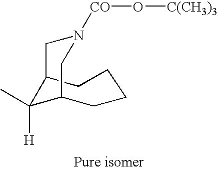 | 173.68, 155.62, 141.76, 133.75,133.41, 133.07, 132.72, 129.26,127.89, 124.09, 121.37, 80.23,61.00, 44.81, 34.22, 33.21,28.93, 28.89, 26.82 |
| 219 | 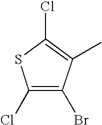 | 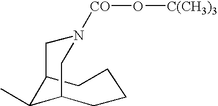 | 173.79, 155.30, 80.49, 45.50,44.28, 37.87, 30.93, 30.63,28.90, 28.83, 27.82, 13.83 |
| 220 | 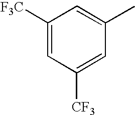 | 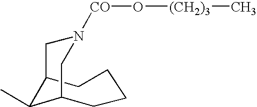 | 171.37, 156.23, 141.64, 133.68,133.41, 133.13, 132.85, 129.34,127.83, 123.81, 121.64, 65.96,51.73, 49.44, 33.21, 32.11,31.48, 26.61, 19.53, 14.03 |
| 221 | 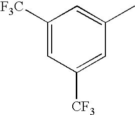 | 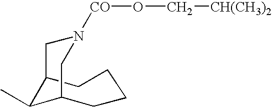 | 171.50, 156.20, 141.72, 133.68,133.40, 133.13, 132.85, 129.33,127.79, 123.82, 121.65, 119.47,72.28, 49.47, 33.23, 32.12,28.41, 26.62, 19.41 |
| 222 | 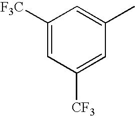 | 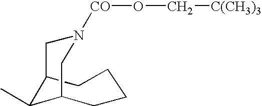 | 171.10, 156.11, 141.55, 133.71,133.44, 133.16, 132.88, 129.41,127.88, 123.81, 121.63, 75.56,49.40, 33.25, 32.12, 31.88,26.87, 26.63 |
| 223 | 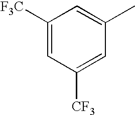 | 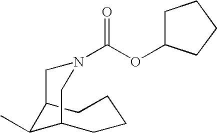 | 171.26, 155.81, 141.52, 133.76,133.41, 133.07, 132.72, 129.40,127.87, 124.06, 121.35, 118.63,51.20, 49.41, 33.29, 32.08,26.60, 23.96 |
| 224 | 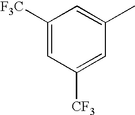 | 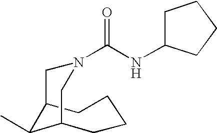 | 173.17, 157.69, 142.62, 133.03,132.69, 129.06, 127.21, 124.20,121.48, 53.07, 51.98, 49.66,34.01, 33.20, 33.12, 32.49,26.63, 24.03 |
| 225 | 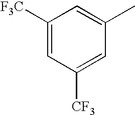 | 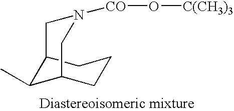 | 171.86, 171.29, 155.31, 155.12,141.65, 133.43, 133.08, 129.35,127.93, 124.07, 121.35, 80.49,80.21, 47.63, 47.30, 28.87,26.44, 19.90, 19.43 |
| 226 | 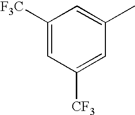 | 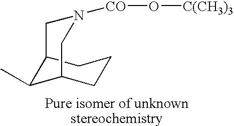 | 155.48, 132.98, 132.64, 132.30,131.96, 127.76, 127.13, 125.79,124.41, 121.70, 118.98, 79.63,48.08, 45.69, 44.59, 40.33,40.12, 32.82, 32.70, 30.55,30.40, 28.88, 20.16 |
| 227 | 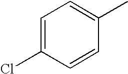 | 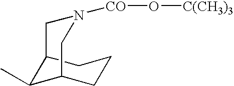 | 171.68, 171.14, 155.27, 155.10,141.23, 137.28, 130.35, 130.26,129.78, 129.73, 80.38, 80.10,47.58, 47.24, 28.89, 26.44,19.94, 19.47 mix |
| 228 | 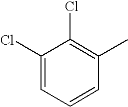 | 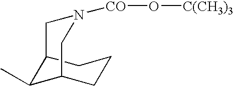 | 171.78, 171.30, 136.09, 136.04,131.99, 131.91, 128.12, 80.34,80.03, 47.73, 47.38, 28.89,26.38, 19.46 |
| 229 | 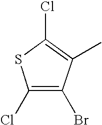 | 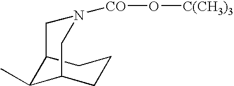 | 172.12, 171.64, 155.11, 131.24,108.50, 80.42, 80.13, 50β.94,47.81, 47.43, 30.43, 28.90,26.49, 19.95, 19.49 |
| 230 | 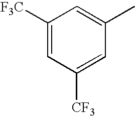 | 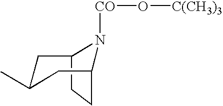 | 171.96, 153.20, 141.06, 133.03,132.69, 128.99, 127.59, 80.04,36.97, 28.45 |
| 231 | 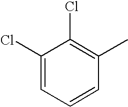 | 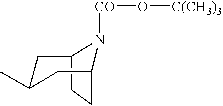 | 174.00, 153.35, 139.11, 135.50,135.37, 131.59, 130.46, 127.77,79.63, 40.66, 40.45, 40.24,40.04, 36.49, 32.90, 28.81 |
| 232 | 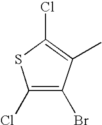 | 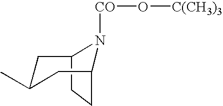 | 172.19, 153.03, 137.04, 130.71,125.99, 108.01, 79.83, 36.68,32.67, 28.48 |
| 233 | 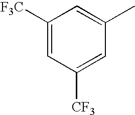 | 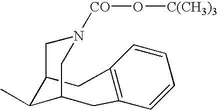 | 170.84, 155.33, 141.38, 138.52,133.61, 133.26, 132.92, 132.57,129.61, 129.42, 127.87, 126.98,124.13, 121.41, 118.69, 80.37,50.56, 49.24, 48.24, 35.17,31.36, 31.05, 28.66 |
| 234 | 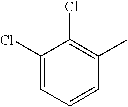 | 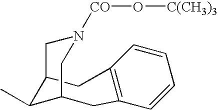 | 171.09, 154.50, 138.81, 138.36,136.07, 135.96, 132.06, 130.53,129.88, 128.35, 128.07, 127.09,126.94, 79.87, 50.88, 48.44,47.60, 36.29, 31.26, 30.97, 28.61 |
| 235 | 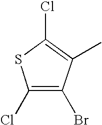 | 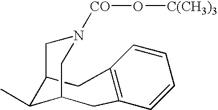 | 171.49, 154.44, 138.78, 138.65,137.68, 131.04, 129.90, 129.38,127.09, 126.90, 126.33, 108.55,79.87, 50.95, 48.37, 47.51,36.45, 31.20, 30.82, 28.61 |
| 236 | 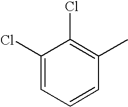 | 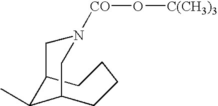 | 273.58, 171.44, 155.56, 155.21,138.38, 136.03, 136.00, 135.85,132.09, 131.85, 130.47, 128.11,128.08, 80.54, 80.23, 49.60,44.82, 33.17, 32.01, 28.89,28.86, 26.83 |
| 237 | 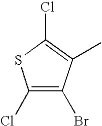 | 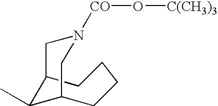 | 171.57, 155.09, 50.39, 49.69,33.15, 32.01, 28.09 |
| 238 | 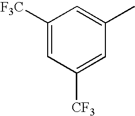 | 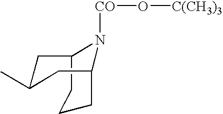 | 173.55, 155.37, 142.08, 133.28,132.94, 129.23, 127.63, 124.13,121.42, 80.83, 45.58, 44.58,37.76, 30.86, 30.55, 29.39,28.87, 27.50, 13.73 |
| 239 | 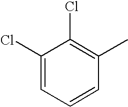 | 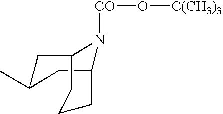 | 172.84, 154.11, 138.23, 136.07,135.96, 131.90, 131.82, 130.46,128.07, 80.03, 46.23, 44.69,39.57, 31.81, 29.31, 28.88,28.84, 20.37 |
| 240 | 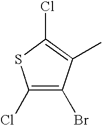 | 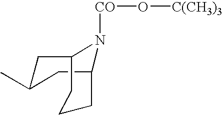 | 173.14, 154.11, 137.49, 131.08,126.35, 108.46, 80.83, 46.20,44.66, 39.61, 31.90, 31.74,29.34, 28.83, 28.86, 20.41 |

Analogously to methods as described in the PROCEDURES (Examples A to Q), but using appropriate starting materials, compounds of formula

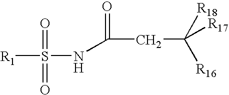

wherein R₁, R₁₆+R₁₇ are as defined in TABLE 4 and R₁₈ is hydrogen or is as defined in TABLE 4 (compounds of formula I, wherein m is 0, n is 1, and R₁ is a group of formula VII) are obtained. If not otherwise indicated in TABLE 4, characterisation data is ¹HNMR data, and ¹³C-NMR and ¹HNMR data are determined in CDCl₃.

TABLE 4

| EX | R₁ | R₁₆ + R₁₇/R₁₈ | m.p./¹H-NMR/¹³C-NMR |
|---|---|---|---|
| 241 | 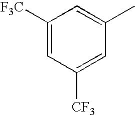 | 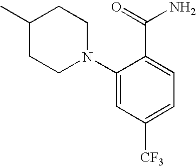 | (DMSO-d₆): δ = 1.25(dq, 2 H);1.59(d, 2 H); 1.70(m, 1 H); 1.97(d, 2 H); 2.66(t, 2 H); 3.12(d,2 H); 7.30(s, 1 H); 7.35(d, 1 H);7.62(s, 1 H); 7.73(d, 1 H); 8.19(s, 1 H); 8.27(s, 1 H); 8.29(s, 2 H). |
| 242 | 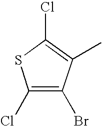 | 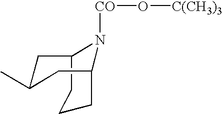 | 170.39, 170.31, 155.44,154.43, 131.45, 126.22,108.68, 79.91, 79.80, 47.36,45.93, 45.86, 45.67, 44.61,42.52, 36.84, 36.46, 32.10,31.95, 31.25, 30.90, 30.08,29.29, 29.17, 28.92, 27.53,20.44, 14.02 |
| 243 | 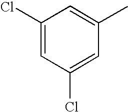 | 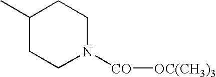 | (DMSO-d₆): 0.92(m, 2 H);1.35(s, 9 H); 1.42(m, 2 H);1.74(m, 1 H); 2.10(d, 2 H);2.54-2.70(m, 2 H); 3.77-3.88(d, 2 H); 7.80(d, 2 H); 7.97(t, 1 H) |
| 244 | 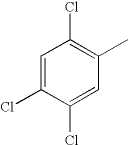 | 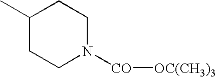 | 1.02-1.15(m, 2 H); 1.44(s,9 H); 1.56-1.68(m, 2 H); 1.83-1.95(m, 1 H); 2.12-2.25(m,2 H); 2.57-2.73((m, 2 H); 3.91-4.10(m, 2 H); 7.56(s, 1 H);8.23(s, 1 H) |
| 245 | 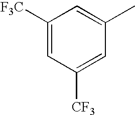 | 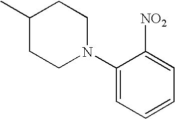 | (DMSO-d₆): 1.20(dq, 2 H);1.51(d, 2 H); 1.73(m, 1 H);2.20(d, 2 H); 2.70(dt, 2 H);3.06(d, 2 H); 7.05(t, 1 H);7.24(d, 1 H); 7.52(t, 1 H);7.74(d, 1 H); 8.41(s, 2 H); 8.53(s, 1 H) |
| 246 | 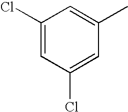 | 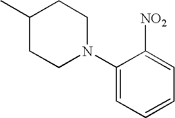 | (DMSO-d₆): 1.09(dq, 2 H);1.43(d, 2 H); 1.63(m, 1 H);2.09(d, 2 H); 2.51(t, 2 H);2.97(d, 2 H); 6.95(t, 1 H);7.14(d, 1 H); 7.42(ddd,1 H); 7.64(dd, 1 H); 7.72(d,2 H); 7.90(t, 1 H) |
| 247 | 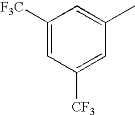 | 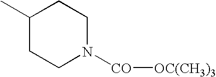 | 1.03-1.14(m, 2 H); 1.44(s,9 H); 1.55-1.65(m, 2 H); 1.88-1.96(m, 1 H); 2.16-2.23(m,2 H); 2.61-2.77(m, 2 H); 3.98-4.10(m, 2 H); 8.12(s, 1 H);8.50(s, 2 H) |
| 248 | 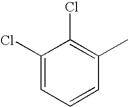 | 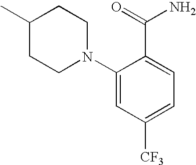 | 247-251° |
| 249 | 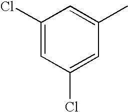 | 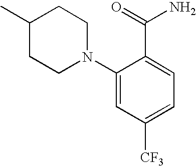 | 195-198° |
| 250 | 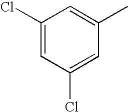 | 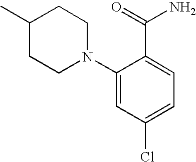 | 149-152° |
| 251 | 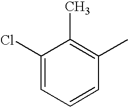 | 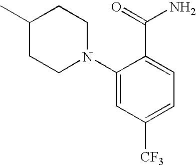 | 243-246° |
| 252 | 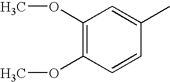 | 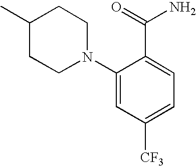 | 179-183° |
| 253 | 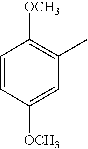 | 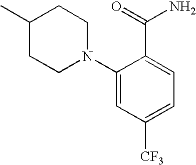 | 92-95° |
| 254 | 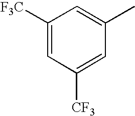 | 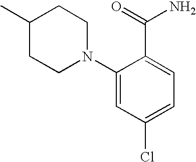 | 81-83° |
| 255 | 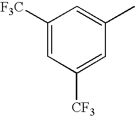 | 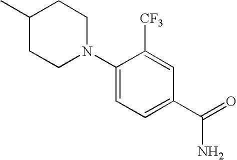 | 150-153° |
| 256 | 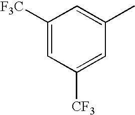 | 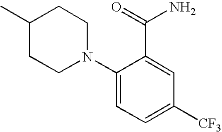 | 174-178° |
| 257 | 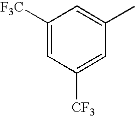 | 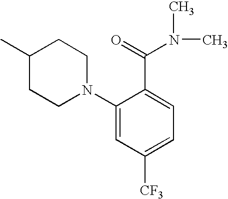 | 129-133° |
| 258 | 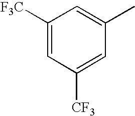 | 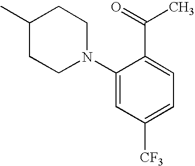 | 93-96° |
| 259 | 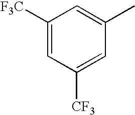 | 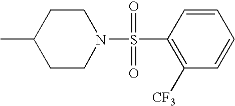 | 1.10(q, 2 H), 1.52-1.61(m,3 H), 1.93(d, 2 H), 2.25(t, 2 H),3.48(d, 2 H), 7.89-7.94(m,2 H), 8.05(broad d, 1 H), 8.12(broad d, 1 H), 9.29(broad s,2 H), 8.30(broad s, 1 H) |
| 260 | 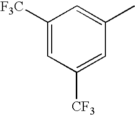 | 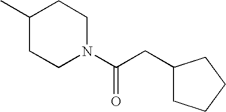 | 98-101° |
| 261 | 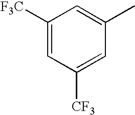 | 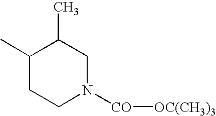 | 170.70, 170.43, 155.84, 155.24,41.82, 141.76, 133.73, 133.38,133.03, 132.69, 129.27, 127.80,126.60, 124.08, 121.37, 80.47,80.32, 43.61, 41.02, 39.59,36.32, 32.34, 28.79, 16.68 |
| 262 | 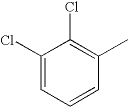 | 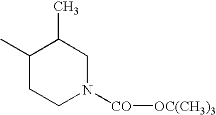 | 170.77, 170.45, 155.71, 155.13,138.41, 135.99, 135.93, 131.90,131.87, 130.57, 130.54, 128.03,80.16, 80.03, 43.61, 40.73,39.54, 36.03, 35.82, 32.22,31.56, 28.82, 26.66, 16.72, 11.66 |
| 263 | 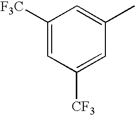 | 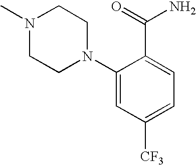 | 160-165° |
| 264 | 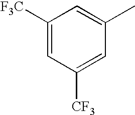 | 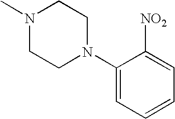 | 140-150° |
| 265 | 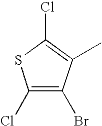 | 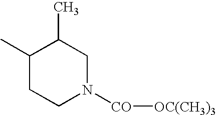 | 170.88, 170.52, 155.65,155.07, 137.33, 137.25,131.35, 126.34, 108.63,108.58, 80.11, 79.96, 40.78,39.51, 36.04, 35.73, 32.25,31.69, 28.83, 16.78 |
| 266 | 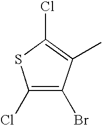 | 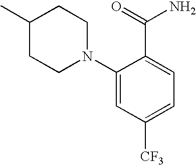 | 153-156° |
| 267 | 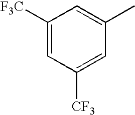 | 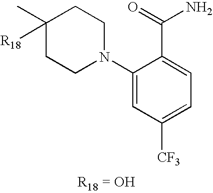 | (DMSO-d₆): 1.42-1.65(m, 4 H),2.85-3.05(m, 4 H), 3.55(s,2 H), 5.72(s, 1 H, OH), 7.32(s,1 H), 7.34(d, 1 H), 7.59 and8.18(2 s, 2 H, NH), 7.72(d,1 H), 8.18(s, 1 H), 8.26(s, 2 H) |
| 268 | 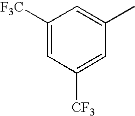 | 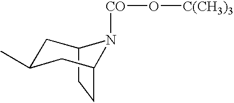 | 170.85, 170.22, 153.88, 142.03,133.25, 132.91, 129.31, 127.60,121.42, 80.45, 43.90, 43.58,35.59, 28.92, 28.81, 28.18,26.72, 25.67 |
| 269 | 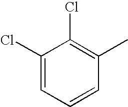 | 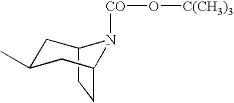 | 170.22, 153.77, 138.56,135.99, 138.88, 131.82,130.62, 128.03, 127.96,80.00, 44.08, 43.57, 28.94,28.86, 26.25, 25.44 |
| 270 | 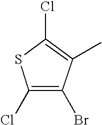 | 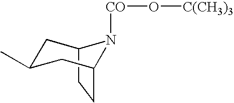 | 170.73, 170.55, 153.81,137.00, 131.56, 108.75,80.13, 44.04, 43.54, 28.97,28.88, 28.26, 26.25, 25.40 |
| 271 | 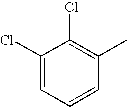 | 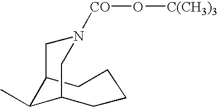 | 170.46, 155.24, 138.35,136.06, 135.99, 131.84, 130.54,128.07, 79.90, 40.33, 39.46,35.56, 31.25, 28.92, 26.67 |
| 272 | 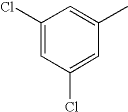 | 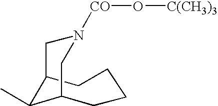 | 170.42, 155.35, 141.71,136.36, 134.41, 127.09,80.05, 40.34, 39.48, 35.60,31.31, 28.92, 26.67 |
| 273 | 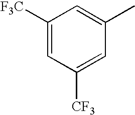 | 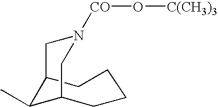 | 170.38, 155.51, 141.74,133.47, 133.19, 129.28,127.91, 123.81, 80.38, 46.00,40.45, 39.53, 35.60, 31.36,28.90, 26.60 |
| 274 | 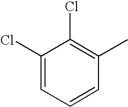 | 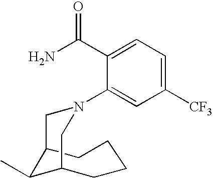 | (CDCl₃/DMSO-d₆): 171.89,170.37, 129.15, 135.54,135.42, 131.74, 130.82,130.56, 127.80, 116.87,61.83, 39.27, 38.78, 36.13,31.29, 26.91 |
| 275 | 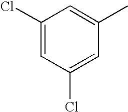 | 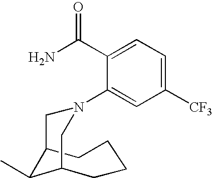 | 170.41, 141.73, 136.35,134.40, 131.01, 127.11,62.23, 38.98, 38.86, 35.89,31.06, 26.83 |
| 276 | 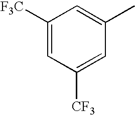 | 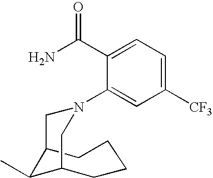 | 170.81, 141.77, 133.41,133.06, 130.83, 129.27,127.88, 62.07, 39.04, 35.97,31.11, 26.84 |
| 277 | 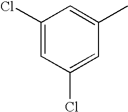 | 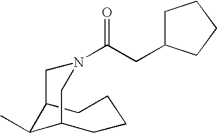 | 173.06, 170.82, 142.22, 136.26,134.16, 127.05, 54.43, 49.85,40.20, 39.81, 39.09, 37.17,35.86, 35.64, 33.19, 31.58,31.43, 26.97, 26.37, 25.37, 25.33 |
| 278 | 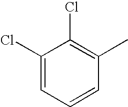 | 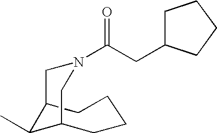 | 172.69, 170.42, 138.53,135.97, 131.79, 130.56,128.05, 54.27, 49.69, 40.18,39.76, 39.14, 37.04, 35.66,33.16, 31.44, 26.99, 26.36,25.37, 25.33 |
| 279 | 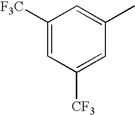 | 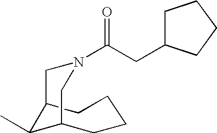 | 173.27, 171.15, 142.24, 133.63,133.28, 132.94, 132.59, 129.18,127.60, 124.14, 121.42,118.70, 54.45, 49.86, 40.19,39.79, 39.02, 37.21, 35.89,35.53, 33.13, 31.58, 31.33,26.93, 26.33, 25.30, 25.25 |
| 280 | 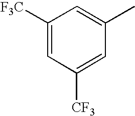 | 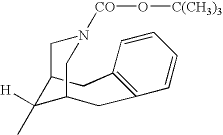 | 171.84, 154.00, 142.66, 139.62,139.35, 133.05, 132.71, 129.95,129.40, 129.01, 127.27, 126.74,126.46, 124.16, 79.03, 48.41,7.62, 40.39, 38.63, 35.96, 33.16,32.74, 30.00, 28.50 |
| 281 | 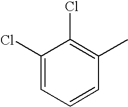 | 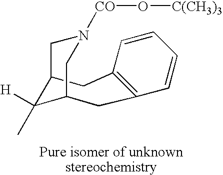 | 171.57, 154.08, 139.84,139.53, 139.15, 135.59,135.45, 131.75, 130.55,129.98, 129.41, 127.84,126.70, 126.45, 79.01, 48.47,47.71, 40.18, 38.51, 36.04,35.99, 33.13, 32.76, 30.04,28.54 |
| 282 | 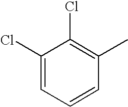 | 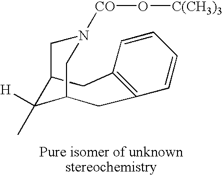 | 169.93, 155.06, 139.30,139.00, 138.41, 136.03,135.98, 131.83, 130.57,129.80, 129.25, 128.09,126.93, 79.70, 42.00, 41.11,39.58, 32.81, 32.40, 28.64 |
| 283 | 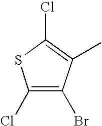 | 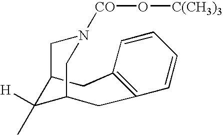 | 171.17, 153.90, 139.116,138.83, 136.06, 131.42,129.54, 128.97, 126.33,126.07, 125.43, 108.28,79.03, 48.00, 47.22, 39.44,38.08, 35.34, 35.32, 32.76,32.19, 29.55, 27.99 |
| 284 | 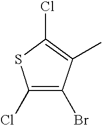 | 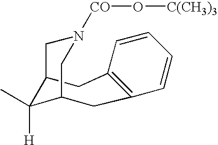 | 170.09, 154.68, 138.96, 138.66,136.71, 131.04, 129.42,128.86, 126.58, 126.48,125.87, 108.28, 79.38, 41.52,41.09, 40.94, 40.71, 39.16,32.38, 32.03, 28.24 |
| 285 | 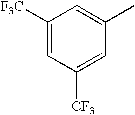 | 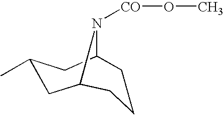 | 170.76, 170.43, 155.94,154.64, 142.05, 141.88,132.96, 132.61, 129.27,127.68, 126.83, 124.13,121.39, 80.36, 80.29,47.50, 46.12, 45.61, 44.94,42.52, 36.93, 36.39, 32.14,31.85, 31.13, 30.88, 30.08,29.42, 29.29, 29.23, 27.81,20.29, 13.87 mix |
| 286 | 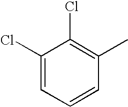 | 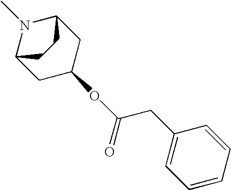 | 239-240° |
| 287 | 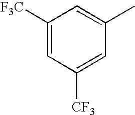 | 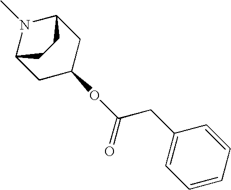 | 85-90° |

Analogously to methods as described in the PROCEDURES (Examples A to Q), but using appropriate starting materials, compounds of formula

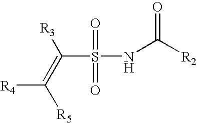

wherein R₂, R₃ and R₄+R₅ are as defined in TABLE 5 (compounds of formula I, wherein m is 0, n is 0, and R₁ is a group of formula II) are obtained.

If not otherwise indicated in TABLE 5 ¹C-NMR and ¹³C-NMR data are determined in CDCl₃.

TABLE 5

| EX | R₂ | R₄ + R₅/R₃ | m.p./¹H-NMR/¹³C-NMR |
|---|---|---|---|
| 288 | 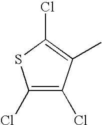 | 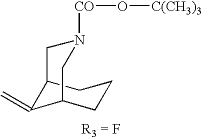 | 158.34, 157.96, 154.94, 144.81, 141.33,137.70, 133.48, 133.13, 129.59, 128.15,124.08, 121.36, 80.53, 50.60, 49.55,49.33, 33.51, 32.01, 31.94, 31.39, 28.85 |
| 289 | 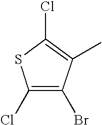 | 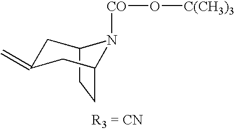 | 153.65, 116.14, 109.03, 80.82, 28.77 |
| 290 | 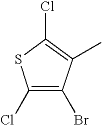 | 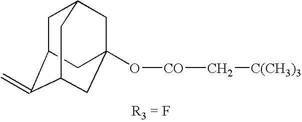 | 171.96, 158.46, 158.09, 145.82, 145.72,139.92, 137.48, 131.21, 126.25, 108.85,78.20, 49.55, 42.06, 41.65, 40.65, 38.38,38.08, 33.12, 33.03, 32.36, 32.34, 31.13,30.38, 30.02 |
| 291 | 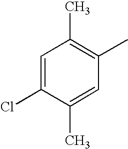 | 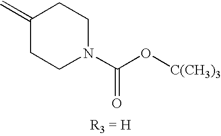 | 1.44(s, 9 H); 2.25(t, 2 H); 2.41(s,3 H); 2.58(s, 3 H); 2.85(t, 2 H); 3.40(t,2 H); 3.48(t, 2 H); 5.62(s, 1 H); 7.30(s,1 H); 8.02(s, 1 H); 8.06(broad, 1 H) |
| 292 | 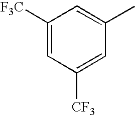 | 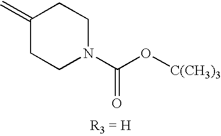 | (DMSO-d₆) 1.25(s, 9 H); 2.02-2.08(m,2 H); 2.56-2.64(m, 2 H); 3.38-3.20(m,4 H); 5.61(m, 1 H); 8.30(s, 2 H); 8.42(s,1 H) |
| 293 | 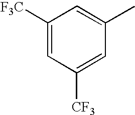 | 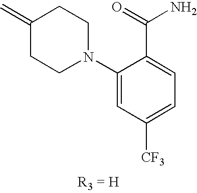 | (DMSO-d₆): 2.40(m, 2 H), 2.91(m, 2 H),3.01(m, 2 H), 3.08(m, 2 H), 5.78(s, 1 H),7.26(s, 1 H), 7.34(d, 1 H), 7.62 and 8.07(2 s, 2 H, NH), 7.66(d, 1 H), 8.45(s, 2 H),8.58(s, 1 H) |
| 294 | 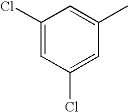 | 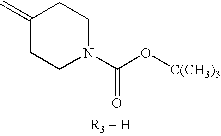 | 1.46(s, 9 H); 2.26(t, 2 H); 2.90(t, 2 H);3.41(t, 2 H); 3.47(t, 2 H); 5.76(s, 1 H);7.56(t, 1 H); 7.90(d, 2 H) |
| 295 | 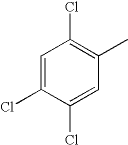 | 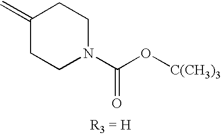 | 1.44(s, 9 H); 2.28(m, 2 H); 2.85(m, 2 H);3.42(m, 2 H); 3.50(m, 2 H); 5.62(s, 1 H);7.63(s, 1 H); 8.18(broad, 1 H); 8.35(s, 1 H) |
| 296 | 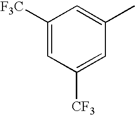 | 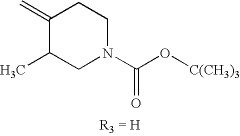 | 168.16, 163.00, 141.84, 133.36, 133.01,129.40, 127.82, 121.40, 112.34, 80.55,28.76 |
| 297 | 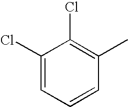 | 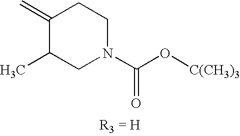 | 167.39, 163.23, 155.07, 138.64, 135.94,135.88, 131.72, 130.71, 127.99, 112.60,80.45, 28.77 |
| 298 | 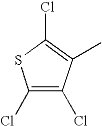 | 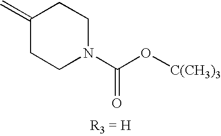 | 169.84, 168.85, 154.55, 154.50, 134.83,122.96, 121.40, 79.32, 43.86, 42.49,28.24, 28.09 |
| 299 | 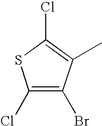 | 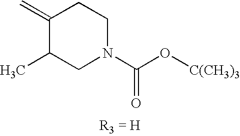 | 167.43, 155.08, 131.89, 126.13, 108.82,80.45, 39.78, 28.78 |
| 300 | 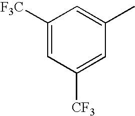 | 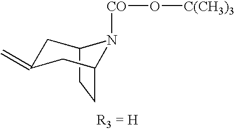 | 162.46, 141.87, 133.34, 133.00, 129.37,127.83, 121.40, 118.03, 80.40, 54.13,30.08, 28.82 |
| 301 | 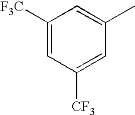 | 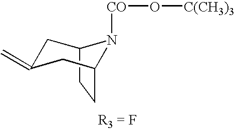 | 153.69, 145.66, 143.194, 141.23,135.04, 134.92, 133.82, 133.47, 133.13,132.78, 129.57, 128.16, 126.78, 124.06,121.34, 80.38, 52.97, 28.80 |
| 302 | 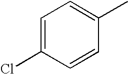 | 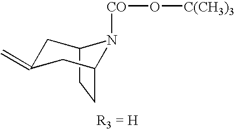 | 162.6, 161.2, 157.6, 141.04, 137.58,130.31, 129.69, 118.37, 80.27, 33.4,31.7, 29.8, 28.83 |
| 303 | 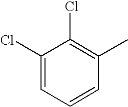 | 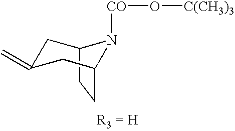 | 161.89, 138.63, 135.92, 131.71, 128.02,118.17, 80.26, 30.08, 28.83 |
| 304 | 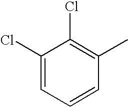 | 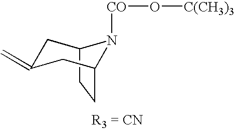 | 127.89, 28.78 |
| 305 | 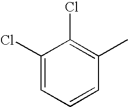 | 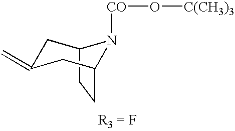 | 153.69, 145.69, 143.35, 138.13, 136.14,136.01, 134.35, 134.22, 131.92, 130.82,128.02, 80.30, 55.01, 28.81 |
| 306 | 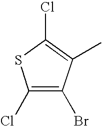 | 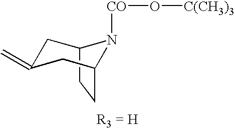 | 136.80, 117.99, 80.31, 54.15, 30.08,28.85 |
| 307 | 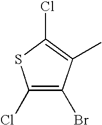 | 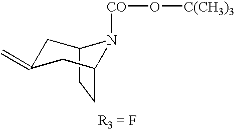 | 145.74, 143.27, 134.69, 126.27, 108.73,80.33, 53.53, 53.13, 28.82 |
| 308 | 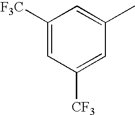 | 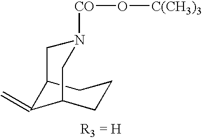 | 172.88, 163.03, 155.29, 141.98, 133.32,132.98, 129.32, 127.75, 126.85, 124.14,121.42, 118.71, 109.95, 80.75, 42.11,28.88, 28.60 |
| 309 | 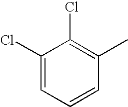 | 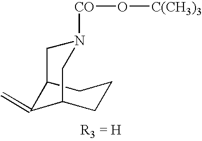 | 171.98, 162.62, 138.27, 135.52, 135.46,131.28, 130.34, 127.63, 109.60, 80.19,51.18, 50.59, 50.29, 49.56, 41.62, 34.52,34.36, 33.65, 33.48, 33.31, 28.48, 19.76 |
| 310 | 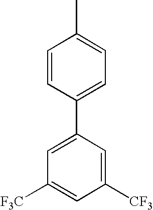 | 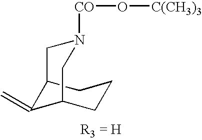 | (DMSO-d₆): 12.11(s, 1 H), 8.35(s, 1 H),8.25(t, J = 1.7 Hz, 1 H), 8.17-8.22(m, 2 H),8.02(dt, J = 1.7 + 8 Hz, 1 H), 7.79(t, J = 8 Hz,1 H), 5.77(s, 1 H), 3.98-4.18(m, 2 H), 3.78(br.s, 1 H), 2.70-2.98(m, 2 H), 2.24(br.s,1 H), 1.52-1.96(m, 6 H), 1.37(s, 9 H) |
| 311 | 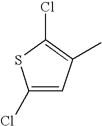 | 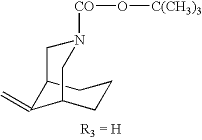 | 172.41, 163.25, 155.17, 134.96, 132.34,127.84, 109.97, 80.50, 51.60, 51.08,50.74, 50.03, 42.07, 34.80, 34.11, 33.91,30.07, 28.89, 20.20 |
| 312 | 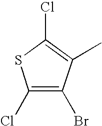 | 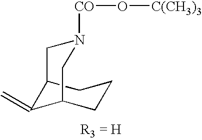 | 170.31, 164.59, 135.38, 132.50, 125.43,110.85, 109.01, 80.05, 51.55, 51.00,50.66, 49.95, 41.73, 34.64, 33.73, 33.56,28.80, 20.16 |
| 313 | 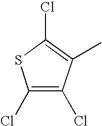 | 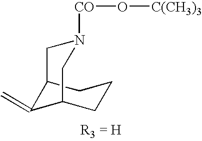 | 169.30, 163.66, 154.10, 133.70, 130.31,122.51, 121.09, 109.85, 79.26, 50.61,50.02, 49.68, 49.01, 40.74, 33.72, 32.70,27.77, 19.17 |
| 314 | 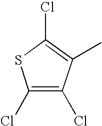 | 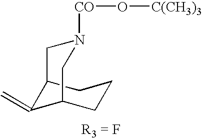 | αD₂₅ = −4.1° (optical rotation)Pure (+) isomer of unknownstereochemestry |
| 315 | 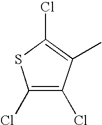 | 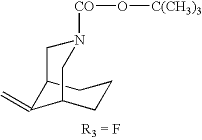 | αD₂₅ = +7.9° (optical rotation)Pure (−) isomer of unknown sterochem. |
| 316 | 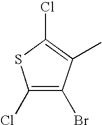 | 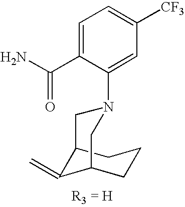 | 171.24, 170.90, 163.49, 150.58, 136.63,134.44, 134.11, 131.78, 131.40, 130.94,126.18, 125.23, 122.52, 119.73, 116.99,111.22, 108.84, 59.63, 58.06, 42.49,34.37, 34.28, 33.44, 19.45 |
| 317 | 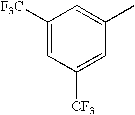 | 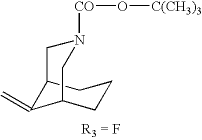 | 144.81, 141.33, 137.70, 133.48, 133.13,129.59, 128.15, 124.08, 121.36, 80.53,50.60, 49.55, 49.33, 33.51, 32.01, 31.94,31.39, 28.85, 19.85 |
| 318 | 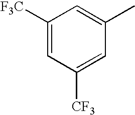 | 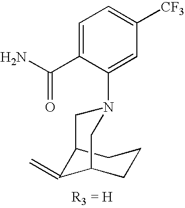 | 171.43, 163.10, 150.47, 142.01, 134.47,133.36, 133.09, 131.31, 130.53, 129.32,127.82, 123.88, 121.70, 117.16, 111.31,59.57, 58.16, 42.39, 34.33, 34.26, 33.32,19.39 |
| 319 | 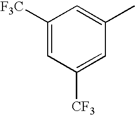 | 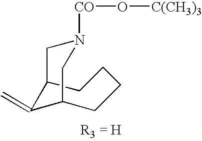 | 169.02, 141.94, 133.36, 133.02, 130.01,128.69, 80.42, 44.05, 36.25, 29.37,29.37, 28.86, 28.32 |
| 320 | 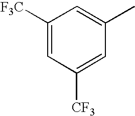 | 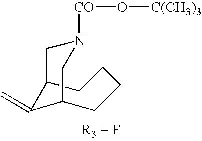 | 157.93, 157.56, 155.27, 144.25, 141.33,140.98, 140.88, 133.81, 133.47, 133.12,132.78, 130.25, 130.04, 129.63, 129.51,129.05, 128.87, 128.60, 128.30, 127.99,126.79, 124.07, 121.36, 118.64, 80.65,49.87, 33.80, 33.72, 33.63, 33.54, 33.20,33.05, 29.54, 29.33, 28.83, 28.30, 28.10 |
| 321 | 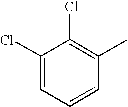 | 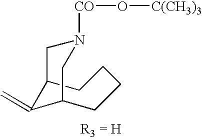 | 167.91, 162.70, 155.31, 138.69, 135.94,135.90, 135.77, 130.72, 128.77, 127.34,80.39, 43.88, 36.17, 36.02, 29.57, 29.37,28.89, 28.38, 28.16 |
| 322 | 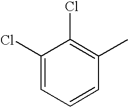 | 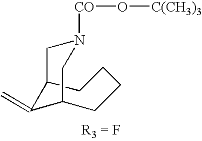 | 155.15, 141.89, 140.47, 140.38, 138.21,136.13, 136.02, 131.87, 130.84, 128.06,80.40, 33.69, 33.61, 33.06, 28.84, 26.64 |
| 323 | 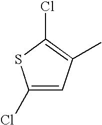 | 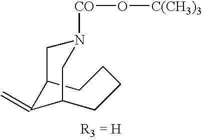 | 168.05, 162.89, 155.36, 134.99, 132.24,127.87, 127.83, 116.30, 80.41, 53.80,49.57, 43.96, 36.17, 30.07, 28.87, 26.73,26.54 |
| 324 | 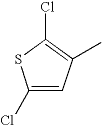 | 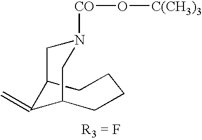 | 155.15, 144.33, 141.86, 140.61, 140.51,134.31, 133.1, 127.96, 127.85, 80.38,33.71, 33.63, 33.11, 32.96, 28.86, 26.68 |
| 325 | 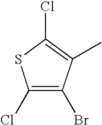 | 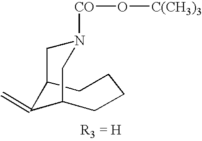 | 168.30, 162.87, 155.31, 136.66, 131.80,126.17, 108.77, 80.40, 43.97, 36.23,36.11, 29.60, 29.38, 28.88, 28.36, 28.14 |
| 326 | 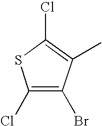 | 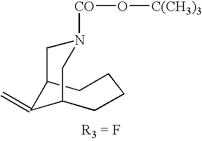 | 157.93, 157.57, 155.18, 144.29, 141.82,140.73, 140.64, 131.16, 126.25, 108.73,80.43, 33.82, 33.73, 33.57, 33.09, 32 |
| 327 | 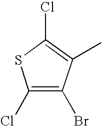 | 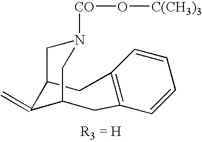 | 163.98, 129.19, 128.90, 126.74, 126.40,114.47, 79.43, 42.71, 42.50, 38.31,33.72, 33.50, 29.53, 28.17, 22.54 |
| 328 | 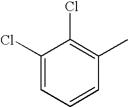 | 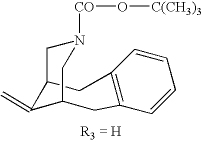 | 162.79, 138.58, 135.83, 131.66, 129.12,127.98, 127.68, 127.37, 115.07, 80.37,43.22, 37.65, 36.81, 28.71 |
| 329 | 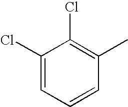 | 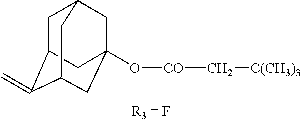 | 172.04, 158.62, 158.25, 145.09, 145.00,140.10, 138.36, 137.65, 135.96, 135.90,130.79, 127.27, 78.30, 49.56, 42.02,40.52, 38.18, 37.10, 33.08, 33.02, 32.33,32.26, 31.11, 30.66, 30.37, 29.93, 29.71 |
| 330 | 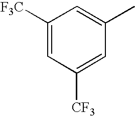 | 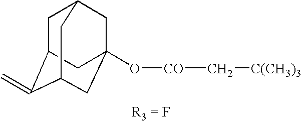 | 172.02, 158.27, 157.91, 141.29, 139.85,137.41, 133.48, 133.14, 132.79, 130.34,49.54, 32.25, 31.13, 30.33, 29.93 |

Analogously to methods as described in the PROCEDURES (Examples A to Q), but using appropriate starting materials, compounds of formula

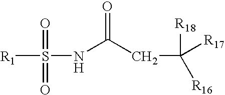

wherein R₁₈ is hydrogen and R₁ and R₁₆+R₁₇ are as defined in TABLE 6 (compounds of formula I, wherein m is 0, n is 1, and R₂ is a group of formula VII) are obtained. If not otherwise indicated ¹³C-NMR and ¹HNMR data in TABLE 6 are determined in DMSO-d₆.

TABLE 6

| EX | R₁ | R₁₆ + R₁₇ | m.p./¹H-NMR/¹³C-NMR |
|---|---|---|---|
| 331 | 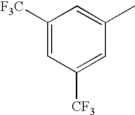 | 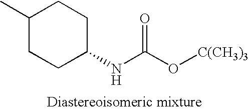 | 93-96° |
| 332 | 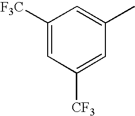 | 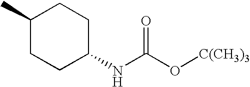 | 0.93(q, 2 H); 1.03(q, 2 H); 1.34(s, 9 H); 1.40-1.50(m, 3 H); 1.65(d, 2 H); 2.07(d, 2 H); 3.07(m,1 H); 4.50(broad, 1 H); 8.12(s,1 H); 8.52(s, 2 H) |
| 333 | 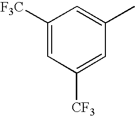 | 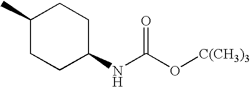 | 1.12-1.28(m, 2 H);1.45(s, 9 H);1.40-1.70(m, 6 H); 1.83-1.94(m1 H); 2.21(d, 2 H); 3.62-3.76(m,1 H); 4.60(broad, 1 H); 5.33(broad, 1 H); 8.12(s, 1 H); 8.50(s, 2 H) |
| 334 | 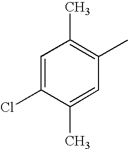 | 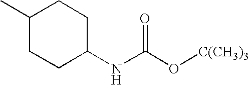 | 0.90(q, 1 H); 1.07(q, 1 H); 1.20-1.52(m, 6 H); 1.37/1.39(s, 9 H);1.63-1.78(m, 1 H); 2.10/2.17(d,2 H); 2.38(s, 3 H); 2.52(s, 3 H);3.10/3.40(m, 1 H); 7.15/7.21(d,1 H); 7.52(s, 1 H); 7.80(s, 1 H);12.18/12.22(s, 1 H) |
| 335 | 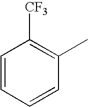 | 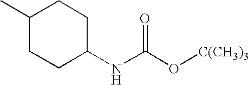 | 0.88(q, 2 H); 1.05(q, 2 H); 1.18-1.54(m, 6 H); 1.36/1.37(s, 9 H);1.63-1.78(m, 1 H); 2.12/2.18(d,2 H); 3.10/3.40(m, 1 H); 6.63/6.70(d, 1 H); 7.88-8.04(m, 3 H);8.30(m, 1 H); 12.36(s, 1 H) |
| 336 | 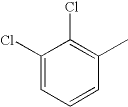 | 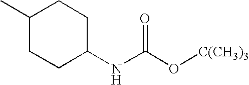 | 0.88(d, 1 H); 1.07(d, 1 H); 1.18-1.53(m, 6 H); 1.36/1.38(s, 9 H);1.64-1.79(m, 1 H); 2.10/2.17(d,2 H); 3.33-3.41(m, 1 H); 6.30(broad, 1 H); 7.56(dt, 1 H); 7.91(dd, 1 H); 8.04(dd, 1 H); 12.3(broad, 1 H) |
| 337 | 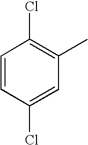 | 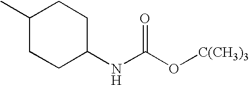 | 0.90(q, 1 H); 1.08(q, 1 H); 1.20-1.30(m, 2 H); 1.30-1.54(m, 4 H);1.37/1.38(s, 9 H); 1.65-1.81(m,1 H); 2.13/2.20(d, 2 H); 3.10/3.40(m, 1 H); 6.63/6.70(d, 1 H); 7.73(d, 1 H); 7.81(d, 1 H); 8.03(s, 1 H) |
| 338 | 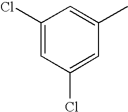 | 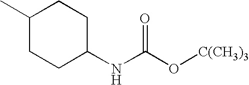 | 0.91(q, 1 H); 1.08(q, 1 H); 1.18-1.32(m, 2 H); 1.36(s, 9 H); 1.35-1.56(m, 3 H); 1.65-1.80(m, 2 H);2.13/2.17(d, 2 H); 3.10/3.41(m,1 H); 6.62-6.73(m, 1 H); 7.85(s,2 H); 8.06(s, 1 H); 12.0(broad, 1 H) |
| 339 | 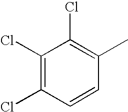 | 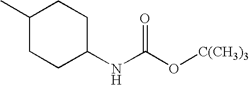 | 1.12(q, 1 H); 1.27(q, 1 H); 1.30-1.50(m, 2 H); 1.56/1.57(s, 9 H);1.60-1.75(m, 3 H); 1.84-2.02(m,2 H); 2.34/2.40(d, 2 H); 3.31/3.61(m, 1 H); 6.85/6.91(d,1 H); 8.13(d,1 H); 8.29(d, 1 H); 12.4(broad, 1 H) |
| 340 | 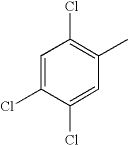 | 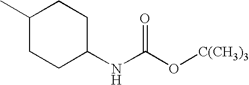 | 0.90(q, 1 H); 1.08(q, 1 H); 1.20-1.32(m, 2 H); 1.37/1.38(s, 9 H);1.35-1.55(m, 3 H); 1.66-1.80(m,2 H); 2.12/2.18(d, 2 H); 3.10/3.40(m, 1 H); 6.64/6.70(d, 1 H); 8.15(s,1 H); 8.16(s, 1H); 12.7(broad, 1 H) |
| 341 | 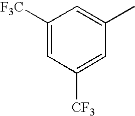 | 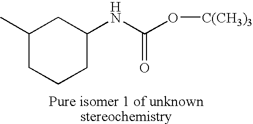 | (CDCl₃): 170.84, 141.87,133.31, 132.97, 132.62, 129.30,127.73, 124.11, 121.39, 47.03,44.35, 38.28, 35.32, 32.48,30.38, 28.80 |
| 342 | 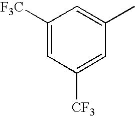 | 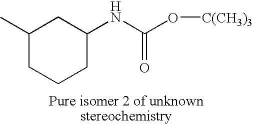 | (CDCl₃): 170.90, 141.79,133.32, 132.97, 129.31, 127.73,124.10, 44.28, 35.90, 32.74,28.78, 28.43, 26.43 |
| 343 | 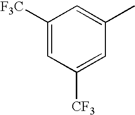 | 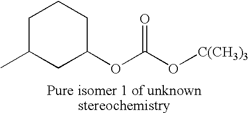 | (CDCl₃): 153.06, 132.95,132.67, 128.63, 127.31, 123.40,121.23, 82.06, 75.40, 43.47,33.48, 31.03, 30.50, 27.78 |
| 344 | 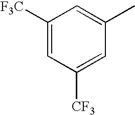 | 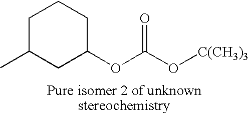 | (CDCl₃): 169.97, 153.49,141.64, 133.73, 133.45, 133.18,132.90, 129.37, 127.94, 123.81,121.64, 82.27, 72.32, 43.62,33.61, 29.49, 28.24, 27.24 |
| 345 | 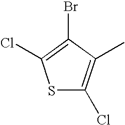 | 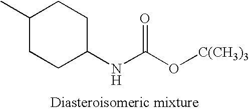 | 0.95(q, 1 H); 1.11(q, 1 H); 1.22-1.36(m, 2 H); 1.38(s, 9 H); 1.40-1.60(m,3 H); 1.68-1.87(m, 2 H); 2.15/2.21(d, 2 H); 3.13/3.44(m, 1 H); 6.73/6.68(d, 1 H); 12.8(broad, 1 H) |
| 346 | 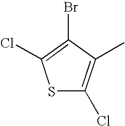 | 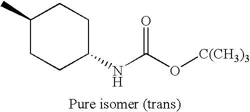 | 0.97(q, 2 H); 1.15(q, 2 H), 1.55-1.68(m, 3 H), 1.77(d, 2 H), 2.18-(d, 2 H), 3.12-3.22(m, 1 H), 6.71(d, 1 H, NH) |
| 347 | 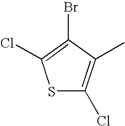 | 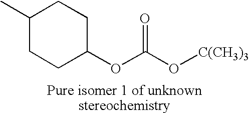 | (CDCl₃): 170.55, 153.54,137.42, 131.23, 126.33, 108.60,82.22, 72.46, 72.40, 43.40,33.39, 29.53, 28.31, 28.24,27.28 |
| 348 | 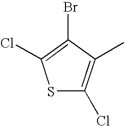 | 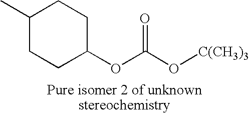 | (CDCl₃): 169.93, 153.01,137.07, 130.76, 129.02, 128.22,126.01, 125.29, 108.13, 81.96,75.37, 42.90, 33.25, 31.09,30.53, 27.80, 21.44 |

Analogously to methods as described in the PROCEDURES (Examples A to Q), but using appropriate starting materials, compounds of formula

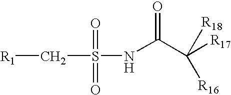

wherein R₁₈ is hydrogen and R₁ and R₁₆+R₁₇ are as defined in TABLE 7 (compounds of formula I, wherein m is 1, n is 0, and R₁ is a group of formula VII) are obtained. If not otherwise indicated in TABLE 7 ¹³C-NMR and ¹HNMR data in TABLE 7 are determined in CDCl₃.

TABLE 7

| EX | R₁ | R₁₆ + R₁₇ | m.p./¹H-NMR |
|---|---|---|---|
| 349 | 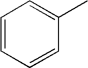 | 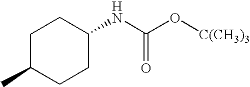 | m.p. = 212-215° |
| 350 | 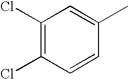 | 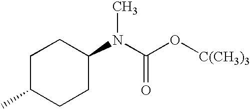 | (DMSO-d₆): 11.52(s, 1 H), 7.70(d, J = 8.4 Hz, 1 H), 7.50(d, J = 2 Hz,1 H), 7.26(dd, J = 8.4 + 2 Hz, 1 H),4.73(s, 2 H), 3.72(br.s, 1 H),2.62(s, 3 H), 2.06-2.14(m,1 H), 1.36-1.80(m, 8 H), 1.37(s, 9 H) |
| 351 | 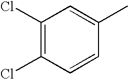 | 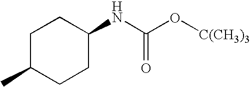 | (DMSO-d₆): 11.33(s, 1 H), 7.68(d, J = 8.3 Hz, 1 H); 7.51(d, J =2 Hz, 1 H), 7.26(dd, J = 2 + 8.3Hz, 1 H), 6.74(br.d, J = 6.6 Hz,1 H), 4.73(s, 2 H), 3.43(br.s,1 H), 2.19-2.28(m, 1 H), 1.40-1.77(m, 8 H), 1.37(s, 9 H) |
| 352 | 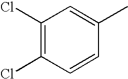 | 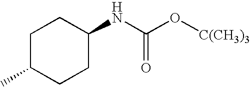 | m.p.: 211-215° |
| 353 | 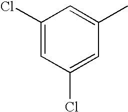 | 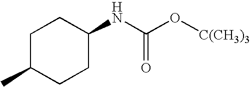 | 8.40(s, 1 H), 7.39(s,1 H), 7.24(s, 2 H), 4.63(s, 2 H), 3.69(br.s, 1 H), 2.30(br.s, 1 H), 1.55-1.78(br.m, 8 H), 1.44(s, 9 H) |
| 354 | 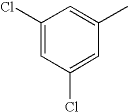 | 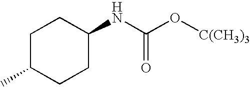 | (DMSO-d₆): 11.50(s, 1 H), 7.66(t, J = 1.9 Hz, 1 H), 7.29(d, J =1.9 Hz, 2 H), 6.68(d, J = 7.8 Hz,1 H), 4.73(s, 2 H), 3.10-3.20(br.s, 1 H), 2.05(tt, J = 3.3 +11.9 Hz, 1 H), 1.63-1.82(m,4 H), 1.28-1.42(m, 2 H), 1.35(s, 9 H), 1.00-1.14(m, 2 H) |
| 355 | 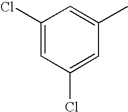 | 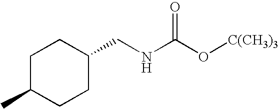 | (DMSO-d₆): 11.49(s, 1 H), 7.66(s, 1 H), 7.29(s, 2 H), 6.78(t, J =5.6 Hz, 1 H), 4.72(s, 2 H), 2.73(t, J = 6.3 Hz, 2 H), 2.08(t, J =11.8 Hz, 1 H), 1.63-1.73(m,4 H), 1.35(s, 9 H), 1.22-1.35(m, 2 H), 0.73-0.86(m, 2 H) |
| 356 | 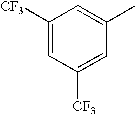 | 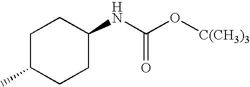 | (DMSO-d₆) 11.52(s, 1 H), 8.18(s, 1 H), 7.95(s, 2 H), 6.66(d, J =7.3 Hz, 1 H), 4.97(s, 2 H), 3.07-3.18(m, 1 H), 2.04(tt, J = 3.2 +8.6 Hz), 1.62-1.80(m, 4 H),1.35(s, 9 H), 1.26-1.35(m,2 H), 0.98-1.11(m, 2 H) |
| 357 | 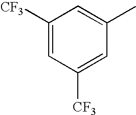 | 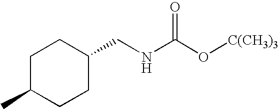 | 204-207 |
| 358 | 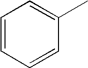 | 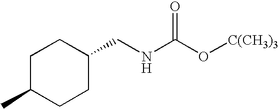 | 0.93(s, 9 H); 1.42(s, 9 H); 1.23-1.62(m, 3 H); 1.78-2.14(m, 5 H);2.98(t, 2 H); 4.58(broad, 1 H);4.64(s, 2 H); 7.26-7.40(m, 5 H);7.58(s, 1 H) |
| 359 | 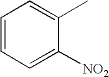 | 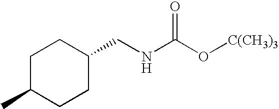 | 0.98(q, 2 H); 1.42(s, 9 H); 1.52-2.20(m, 8 H); 2.99(t, 2 H); 4.59(broad, 1 H); 5.24(s, 2 H); 7.40-7.65(m, 3 H); 8.01(d, 1 H); 8.14(s, 1 H) |
| 360 | 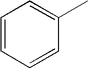 | 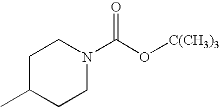 | 1.42(s, 9 H); 1.40-1.78(m, 4 H);2.21(m, 1 H); 2.92(t, 2 H); 4.06(d, 2 H); 4.68(s, 2 H); 7.30-7.40(m, 5 H); 7.75(s, 1 H) |
| 361 | 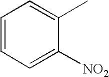 | 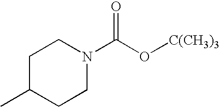 | 1.44(s, 9 H); 1.45-1.90(m, 4 H);2.33(m, 1 H); 2.78(t, 2 H); 4.10(d, 2 H); 5.22(s, 2 H); 7.42-7.70(m, 3 H); 7.92(broad, 1 H); 8.03(d, 1 H) |

Analogously to methods as described in the PROCEDURES (Examples A to Q), but using appropriate starting materials, compounds of formula

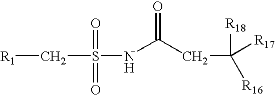

wherein R₁₈ is hydrogen and R₁ and R₁₆+R₁₇ are as defined in TABLE 8 (compound of formula I, wherein m is 1, n is 1, and R₂ is a group of formula VII) are obtained.

TABLE 8

| EX | R₁ | R₁₆ + R₁₇ | ¹HNMR |
|---|---|---|---|
| 362 | 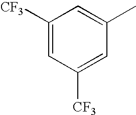 | 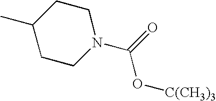 | (DMSO-d₆): 11.63(s, 1 H), 8.18(s,1 H), 7.99(s, 2 H), 5.00(s, 2 H), 3.86(d, J = 12.7 Hz, 2 H), 2.67(br.s, 1 H),2.13(d, J = 7 Hz, 2 H), 1.76-1.89(m, 1 H), 1.50-1.60(m, 2 H), 1.37(s, 9 H), 0.88-1.03(m, 2 H) |

Analogously to methods as described in the PROCEDURES (Examples A to Q), but using appropriate starting materials, compounds of formula

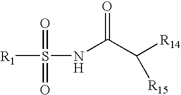

wherein R₁, R₁₄ and R₁₅ are as defined in TABLE 9 (compounds of formula I, wherein m is 0, n is 0, and R₁ is a group of formula VI) are obtained. If not otherwise indicated ¹³C-NMR and ¹HNMR data in TABLE 9 are determined in DMSO-d₆.

TABLE 9

| EX | R₁₄ | R₁₅ | R₁ | m.p./¹HNMR |
|---|---|---|---|---|
| 363 | 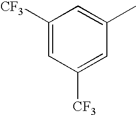 | 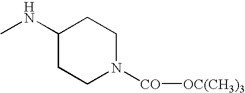 | 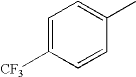 | 150-154° |
| 364 | 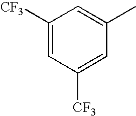 | 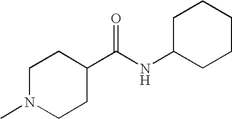 | CF₃ | 171-175° |
| 365 | 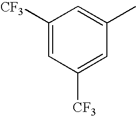 | 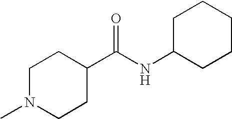 | 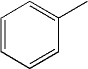 | 169-171° |
| 366 | 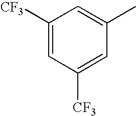 | 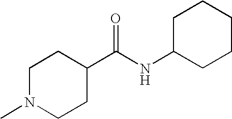 | 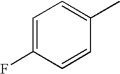 | 140-145° |
| 367 | 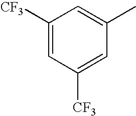 | 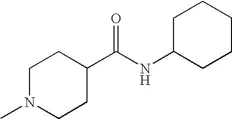 | 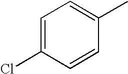 | 229-231°Racemate |
| 368 | 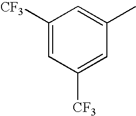 | 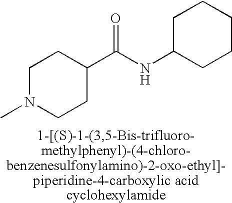 | 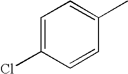 | 9.7(s, br NH), 8.19(s, 1 H),8.0(s, 2 H), 7.73(d, J = 8 Hz,NH), 7.5(d, J = 8.5 Hz, 2 H),7.37(d, J = 8.5 Hz, 2 H),4.95(s, 1 H), 3.46(m, 2 H),2.85(m, 2 H), 2.71(m, 1 H),2.27(m, 1 H), 1.85(m, 3 H),1.67(m, 4 H), 1.53(m, 1 H),1.16(m, 6 H) |
| 369 | 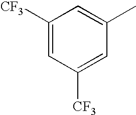 | 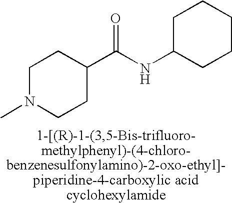 | 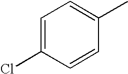 | 9.76(s, br, NH), 8.19(s, 1 H),8.08(s, 2 H), 7.73(d, J = 8 Hz,NH), 7.54(d, J = 8.5 Hz, 2 H),7.37(d, J = 8.5 Hz, 2 H), 4.95(s, 1 H), 3.46(m, 2 H), 2.85(m, 2 H), 2.71(m, 1 H), 2.27(m, 1 H), 1.85(m, 3 H), 1.67(m, 4 H), 1.53(m, 1 H), 1.16(m, 6 H) |
| 370 | 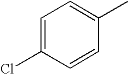 | 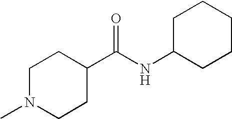 | 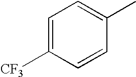 | 250-254° |
| 371 | 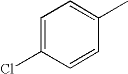 | 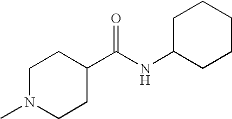 | 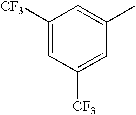 | 254-257° |
| 372 | 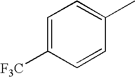 | 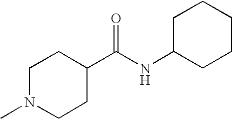 | 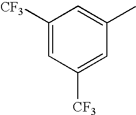 | 249-251° |
| 373 | 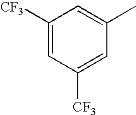 | 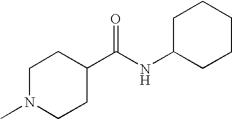 | 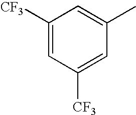 | 7.89(s, br, 3 H), 7.72(d,J = 8.1 Hz, 2 H), 7.63(d,J = 8.2 Hz, 2 H), 7.53(s, br,1 H), 3.85(s, br, 1 H),3.47(m, 1 H), 2.77(s, 1 H),2.50(s, br, 1 H), 1.99(s,br, 2 H), 1.88(s, br, 1 H),1.65(m, 4 H), 1.52(m,4 H), 1.21(m, 3 H), 1.16(m, 3 H) |

Analogously to methods as described in the PROCEDURES (Examples A to Q), but using appropriate starting materials, compounds of formula

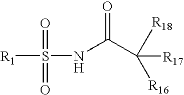

wherein R₁, R₁₆+R₁₇ and R₁₈ are as defined in TABLE 10 (compounds of formula I, wherein m is 0, n is 0, and R₂ is a group of formula VII) are obtained

TABLE 10

| EX | R₁₆ + R₁₇ | R₁₈ | R₁ | ¹HNMR/¹³C-NMR |
|---|---|---|---|---|
| 374 | 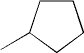 | 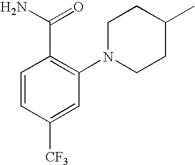 | 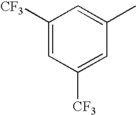 | 175.20, 168.92, 152.57,135.26, 134.93, 133.67,133.33, 132.98, 132.83,132.63, 129.88, 129.27,127.71, 126.82, 125.06,124.10, 122.35, 121.99,121.38, 117.92, 59.79,54.81, 43.10, 32.94,28.94, 25.10 |
| 375 | 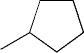 | 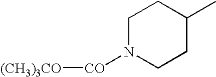 | 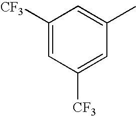 | 174.98, 155.00, 141.65,133.42, 133.07, 129.25,127.83, 121.33, 80.13,59.57, 44.31, 44.10,32.40, 28.77, 28.11,25.45 |

Analogously to methods as described in the PROCEDURES (Examples A to Q), but using appropriate starting materials, compounds of formula

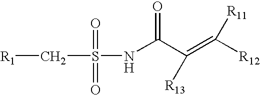

wherein R₁₃ is hydrogen and R₁ and R₁₁+R₁₂ are as defined in TABLE 11 (compounds of formula I, wherein m is 1, n is 0, and R₂ is a group of formula V) are obtained.

TABLE 11

| | | | |
|---|---|---|---|
| EX | R₁₁ + R₁₂ | R₁ | ¹HNMR |

| | | | |
|---|---|---|---|
| 376 | 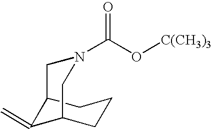 | 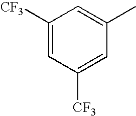 | (CDCl₃): 7.92(s, 1 H), 7.83(s, 2 H), 7.50(br.s, 1 H), 5.46(s, 1 H), 4.81(s, 2 H),4.04-4.42(m, 2 H), 2.92-3.13(m,2 H), 1.40-.30(m, 8 H) 1.46(s, 9) |

Analogously to methods as described in the PROCEDURES (Examples A to Q), but using appropriate starting materials, compounds of formula

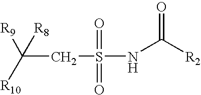

wherein R₈ is hydrogen or is as defined in TABLE 12 and R₂ and R₉+R₁₀ are as defined in TABLE 12 (compounds of formula I, wherein m is 0, n is 1, R₁ is a group of formula VII) are obtained.

TABLE 12

| EX | R₉ + R₁₀ | R₂ | m.p./¹HNMR |
|---|---|---|---|
| 377 | 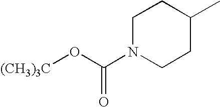 | 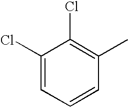 | (DMSO-d₆): 1.12(dq, 2 H), 1.40(s, 9 H),1.85(dd, 2 H), 2.03(m, 1 H), 2.65-2.71(m, 2 H), 3.07(d, 2 H), 3.87(broad d,2 H), 7.29(dd, 1 H), 7.32(dd, 1 H), 7.51(dd, 1 H) |
| 378 | 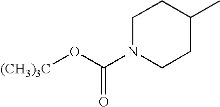 | 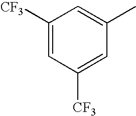 | (DMSO-d₆): 8.45(s, 2 H), 8.12(s, 1 H),3.80(br.d, J = 12.5 Hz, 2 H), 2.46(d, J =6.3 Hz, 2 H), 2.70(br. s, 2 H), 1.90-1.98(m, 1 H), 1.80(br.d, J = 13.3 Hz,2 H), 1.00-1.12(m, 2 H) |
| 379 | 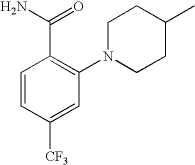 | 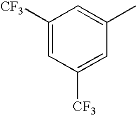 | m.p.: 268-273° |
| 380 | 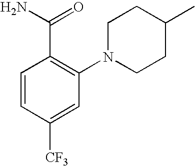 | 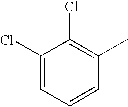 | m.p.: 173-176° |
| 381 | 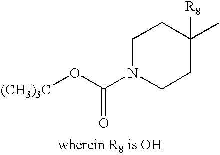 | 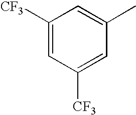 | m.p.: 154-159° |
| 382 | 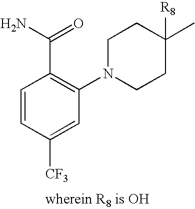 | 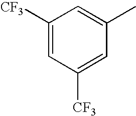 | (DMSO-d₆): 1.38(s, 9 H), 1.59(d, 2 H),1.70(m, 2 H), 3.05(broad, 2 H), 3.35(s,2 H), 3.60(broad d, 2 H), 4.91(s, 1 H,OH), 8.18(s, 1 H), 8.46(s, 2 H) |
| 383 | 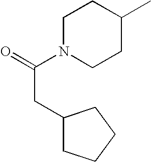 | 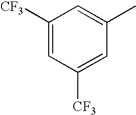 | (CDCl₃): 2 rotamers, selected signals:11.30(br.s, 1 H), 8.62(s, 2 H), 8.08(s,1 H), 4.60 + 3.95(2 × br.d, J = 13 Hz, 2 ×1 H), 3.16 + 3.13(2 × d, J = 12 Hz, 2 H),2.63(t, J = 12 Hz, 1 H) |
| 384 | 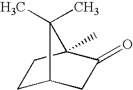 | 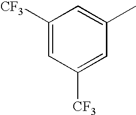 | (DMSO-d₆): 0.78(s, 3 H), 1.04(s, 3 H),1.32(m, 1 H), 1.40(m, 1 H), 1.84-1.92(m, 2 H), 1.97 m, 1 H), 2.29(m, 1 H), 2.62(m, 1 H), 3.26 and 3.47(AB, 2 H), 8.15(broad, 1 H), 8.48(broad, 2 H) |

Analogously to methods as described in the PROCEDURES (Examples A to Q), but using appropriate starting materials, compounds of formula

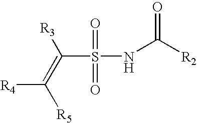

wherein R₃ is hydrogen, and R₂ and R₄+R₅ are as defined in TABLE 13 (compounds of formula I, wherein m is 0, n is 0, R₁ is a group of formula II, and R₂ is (C₆₋₁₈)aryl), are obtained.

TABLE 13

| EX | R₄ + R₅ | R₂ | ¹H-NMR |
|---|---|---|---|
| 385 | 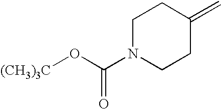 | 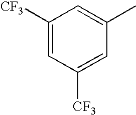 | (DMSO-d₆): 1.42(s, 9 H), 2.33(t, 2 H),2.82(t, 2 H), 3.44(broad, 4 H), 6.61(s,1 H), 8.41(s, 1 H), 8.57(s, 2 H) |
| 386 | 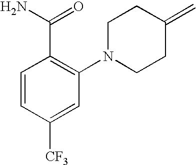 | 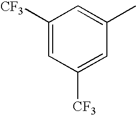 | (DMSO-d₆): 2.40(m, 2 H), 2.93-3.10(m,6 H), 6.44(s, 1 H), 7.27(s, 1 H), 7.36(d,1 H), 7.66(s, 1 H), 7.70(s, 1 H), 8.15(d,2 H, NH), 8.48(s, 2 H) |

1. A composition comprising an ascomycin and a steroid sulfatase inhibitor.

2-3. (canceled)

4. A method of treating inflammatory disorders comprising administering a therapeutically effective amount of a combination of a steroid sulfatase inhibitor with an ascomycin to a subject in need of such treatment.

5. A pharmaceutical composition comprising, in association with at least one pharmaceutically acceptable excipient, a pharmaceutically effective amount of at least one steroid sulfatase inhibitor in combination with at least one ascomycin.

6. A composition according to claim 1 wherein said ascomycin is a compound of formula

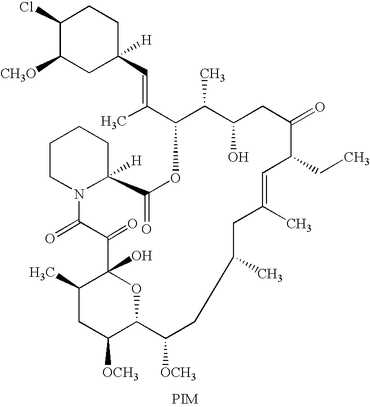

7. A composition according to claim 1 wherein said steroid sulfatase inhibitor is a compound of formula [chemical structure]
